(19) United States
(12) Patent Application Publication
(10) Pub. No.: US 2010/0015148 A1
(43) Pub. Date: Jan. 21, 2010

(54) METHODS AND COMPOSITIONS FOR THE UTILIZATION AND TARGETING OF OSTEOMIMICRY

(75) Inventors: Leland W. K. Chung, Atlanta, GA (US); Wen-Chin Huang, Atlanta, GA (US); Valerie Odero-Marah, Tucker, GA (US); Daqing Wu, Atlanta, GA (US); Chia-Ling Hsieh, Decatur, GA (US); Haiyen Zhau, Atlanta, GA (US)

Correspondence Address: OSHA LIANG L.L.P., TWO HOUSTON CENTER, 909 FANNIN, SUITE 3500, HOUSTON, TX, 77010, US

(73) Assignee: DA ZEN GROUP, LLC, Atlanta, GA (US)

(21) Appl. No.: 12/343,063

(22) Filed: Dec. 23, 2008

Related U.S. Application Data
(63) Continuation-in-part of application No. 11/248,337, filed on Oct. 13, 2005, now abandoned.
(60) Provisional application No. 60/618,452, filed on Oct. 13, 2004.

(51) Int. Cl. A61K 39/395; C12N 5/02; A61K 31/7088; C12Q 1/68

(52) U.S. Cl. 424/138.1; 435/375; 514/44 R; 435/6

(57) ABSTRACT

A method for interfering with osteomimetic properties of a cell includes introducing into the cell an osteomimecry-interfering compound, wherein said osteomimecry-interfering compound prevents or ameliorates the expression of the osteomimetic properties of said cell. A method for treating or ameliorating an osteotropic-related cancer or disorder in a subject includes administering to the subject an osteomimecry interfering compound. A method for identifying a compound that modulates the osteomimetic potential of a cell includes contacting a cell exhibiting osteomimetic potential with a test compound; measuring expression levels of one or more osteomimetic gene products in the cell in the presence and in the absence of the test compound; and identifying a compound that modulates the osteomimetic potential, wherein the compound changes the expression levels of one or more osteomimetic gene products in the cell.

### CROSS-REFERENCE TO RELATED APPLICATIONS

This application is a continuation-in-part of U.S. patent application Ser. No. 11/248,337, filed Oct. 13, 2005, which claims priority under 35 USC Section 119(e) of U.S. Provisional Application No. 60/618,452, filed Oct. 13, 2004, the contents of which are hereby incorporated by reference in their entirety.

### BACKGROUND OF INVENTION

1. Field of the Invention

The present invention generally relates to compositions and methods for treating cancers or disorders with osteomimicry.

2. Background Art

Bone is the second most common site of human cancer metastasis, harboring over 80% of the metastases from prostate, lung, breast and renal cancers. In 2004, bone metastases accounted for two thirds of an estimated 560,000 cancer deaths in the United States with more than 85% of these patients having skeletal metastasis at autopsy. Bone metastases are lethal and often develop after patients fail hormonal therapy. Consistent with this idea, previous work showed that there was no increase of survival for men with hormonal refractory prostate cancer treated with conventional hormone therapy, chemotherapy, or radiation therapy. To date, there is no effective therapy to treat bone metastasis. Therefore, new and effective modalities for treating cancer bone metastasis are urgently needed.

Osseous involvement has been correlated directly with patient survival and the quality of life of cancer patients with bone pain, cancer-associated bone fractures and spinal compression, bone-metastasis-evoked cranial neuropathy from base of skull syndromes, anemia and infection. By targeting the bone, transient successes are achieved. For example, treating prostate and breast cancer patients undergoing hormone withdrawal therapy with bisphosphonate helps reduce bone pain and skeletal complications by inhibiting bone turnover. Strontium 89 combined with chemotherapy increases survival in patients with hormone refractory prostate cancer. Atrasentan, an endothelin-1 receptor antagonist, and thalidomide, an angiogenic inhibitor, are both used clinically to treat cancer bone metastasis. Other approaches include the use of FDA-approved drugs zoledronic acid (Zometa) for treating osteoblastic/osteolytic bone metastases in patients with breast and prostate cancers treated with hormonal therapy, bone-directed chemotherapy, and radiation therapy using strontium-89 or samarium-153. Chemotherapy modalities show promise for reducing the overall incidence of skeletal complications and improving survival in selected groups of hormone-refractory prostate and breast cancer patients.

These promising approaches are generally supported by laboratory results using gene therapy approaches to co-target tumor and stroma and drug therapy targeting osteoblasts, osteoclasts, marrow stromal cells, bone derived endothelium, cell adhesion to extracellular matrices or selected growth factor pathway, all of which show promise in a large number of bone metastasis models. Despite the limited clinical success, however, cancer inevitably recurs and is resistant to treatments.

In view of the above, it remains important to pursue new molecular pathways that can be used to improve prognosis and treatment of cancer patients with lethal cancer phenotypes, bone metastases and associated complications. The present invention addresses a long-felt need for safe and effective methods for treating cancers and disorders with bone metastasis.

### SUMMARY OF INVENTION

One aspect of the invention relates to methods for interfering with osteomimetic properties of a cell. A method in accordance with one embodiment of the invention includes introducing into the cell an osteomimecry-interfering compound, wherein said osteomimecry-interfering compound prevents or ameliorates the expression of the osteomimetic properties of said cell.

A method for treating or ameliorating an osteotropic-related cancer or disorder in a subject, comprising administering to the subject an osteomimecry interfering compound. A method for identifying a compound that modulates the osteomimetic potential of a cell includes contacting a cell exhibiting osteomimetic potential with a test compound; measuring expression levels of one or more osteomimetic gene products in the cell in the presence and in the absence of the test compound; and identifying a compound that modulates the osteomimetic potential, wherein the compound changes the expression levels of one or more osteomimetic gene products in the cell.

Another aspect of the invention relates to methods for treating or ameliorating an osteotropic-related cancer or disorder in a subject. A method in accordance with one embodiment of the invention includes administering to the subject an osteomimecry interfering compound.

Another aspect of the invention relates to methods for identifying a compound that modulates the osteomimetic potential of a cell. A method in accordance with one embodiment of the invention includes contacting a cell exhibiting osteomimetic potential with a test compound; measuring expression levels of one or more osteomimetic gene products in the cell in the presence and in the absence of the test compound; and identifying a compound that modulates the osteomimetic potential, wherein the compound changes the expression levels of one or more osteomimetic gene products in the cell.

Other aspects and advantages of the invention will be apparent from the following description and the appended claims.

### BRIEF DESCRIPTION OF DRAWINGS

FIG. 1 shows immunohistochemical staining of OC and BSP in human primary and bone metastatic prostate cancer tissue specimens, conditioned media stimulate hOC and hBSP promoter activities, and the steady-state levels of OC and BSP mRNA expression in human prostate cancer cell lines. A, Positive and strong OC and BSP stains are detected in both primary and bone metastatic clinical prostate cancer specimens (bold arrows). Some areas of the prostate cancer cells are found lightly or not stained at all (arrow heads). Osteomimicry exists in prostate cancer cells when it is present in primary. Magnification×75. B, CM are collected from human prostate cancer cell lines (LNCaP, C4-2B, DU145, PC3 and ARCaP), a normal human osteoblastic cell line (Kees II), and a human osteosarcoma cell line (MG63). The hOC promoter-reporter construct is co-transfected with CMV promoter-driven β-galactosidase plasmid (for the correction of transfection efficiency as an internal control) into an androgen-independent and metastatic LNCaP cell subline, C4-2B. CM induced hOC promoter activity in a dose-dependent manner (total protein concentration ranged 0-15 μg/ml). C, ARCaP CM also stimulates hBSP promoter activity in a dose-dependent manner (total protein concentration ranged 0-15 μg/ml). D, hOC and hBSP promoter-reporter activities are determined in LNCaP, C4-2B, DU145, PC3, ARCaP and MG63 cell lines in the presence or absence of ARCaP CM (15 μg/ml). hOC and hBSP promoter activities are dramatically elevated by ARCaP CM in LNCaP and C4-2B cells. Fold induction is calculated from the promoter activities assayed in the presence or absence of CM. Data are expressed as the mean±S.D. of three independent experiments with duplicate assays in each experiment. Significant differences of the fold inductions of hOC or hBSP reporter activity are observed by the addition of ARCaP CM: **, p<0.005. E, RT-PCR is performed using total RNAs isolated from LNCaP, C4-2B, PC3 and MG63 cells in the absence (−) or presence (+) of ARCaP CM (15 μg/ml of total protein) for a 12 h incubation period. Expression of the housekeeping gene GAPDH is used as a loading control. The relative expression values of OC and BSP mRNA, normalized by the amounts of GAPDH mRNA expression, are measured by Gel Doc gel documentation software (Bio-Rad). Fold induction represents the ratios of ARCaP CM-treated versus vehicle-treated control of each cell line.

FIG. 2 shows that the cAMP-responsive element (CRE) is responsible for the hOC and hBSP promoter activation induced by ARCaP CM. A, Deletion analysis of hOC promoter. Three cis-elements, AV, OSE2 and OSE1 (9) are not critical for the hOC promoter activation regulated by ARCaP CM (The basal luciferase activities, expressed as RLA, in control hOC/Luc and ΔAV/OSE2/OSE1, were 1440.+−.58 and 1160.+−.200, respectively). ARCaP CM-mediated hOC promoter reporter activity is not affected by the elimination of these three cis-elements. B, Deletion of CRE element abrogates the ARCaP CM-mediated activation of hOC promoter reporter activity. Region A (374 bp), upstream from AV element, contains three cis-acting elements, Tst-1 (−848 to −834), CRE (−643 to −636) and IRF-1 (−609 to −597). Deletion of region A (Δ.A) mutant in hOC promoter dramatically decreases the CM-mediated activation of the promoter activity. Subsequently, ΔTst-1, ΔCRE, and ΔIRF-1 mutant constructs are generated from the hOC promoter using the recombinant PCR method. Only the ACRE construct abolished ARCaP CM-induced hOC promoter activity. The relative activities of various hOC mutation reporter constructs are determined in the presence or absence of ARCaP CM (minus ARCaP CM of the hOC/Luc promoter activity is designed as 1.0). Significant differences of the relative luciferase activity are indicated: *, p<0.05; **, p<0.005. C, Two putative CRE sites are cooperatively regulated in the hBSP promoter activity by ARCaP CM. Single deletion of CRE1 ΔCRE1, −79 to −72) or CRE2 ΔCRE2, −674 to −667) in hBSP promoter reduces partially the promoter activation; the double deletion ΔCRE2/CRE1 construct markedly decreased the ARCaP CM-induced hBSP promoter activity (the hBSP/Luc promoter activity is assigned as 1.0 in the absence of ARCaP CM). Significant differences are calculated: *, p<0.05; **, p<0.005. Data are expressed as the mean±S.D. from three independent studies with duplicate assays in each experiment. D, Point-mutation constructs of the CRE site within hOC promoter are constructed using the QuikChange Site-Directed Mutagenesis Kit (Stratagene, see Materials and Methods). Relative activities of hOC mutation reporter constructs are determined and compared to the hOC/Luc construct (assigned as 1.0 without adding ARCaP CM). Data are expressed as the mean±S.D. from three independent studies with duplicate assays in each experiment. Significant differences from the relative luciferase activity of hOC/Luc: *, p<0.05; **, p<0.005.

FIG. 3 shows that hOC and hBSP promoter activities are stimulated by dibutyryl cAMP (db cAMP) and forskolin (FSK) in a dose-dependent manner. C4-2B cells are co-transfected with A, hOC or B, hBSP promoter plus CMV/β-galactosidase plasmid (an internal control plasmid for transfection efficiency). The transiently transfected cells are treated with different concentrations of db cAMP (from 10⁻⁶ M to 10⁻³ M), FSK (from 10⁻⁸ M to 10⁻⁵ M) or ARCaP CM (15 μg/ml) for 16 h. hOC and hBSP promoter activities are induced by these pharmacologic reagents in a concentration-dependent manner through the activation of cAMP-dependent PKA pathway. Fold induction represents the mean±S.D. of three separate studies with duplicate assays in each experiment. Significant differences from control: *, p<0.05; **, p<0.005. C, RT-PCR is performed using 5 μg of total RNAs isolated from LNCaP, C4-2B, PC3 and MG63 cell lines in the absence (−) or presence (+) of FSK (10⁻⁶ M) exposure for 12 h. The relative expression values of OC and BSP mRNA, normalized by the amounts of GAPDH mRNA, are measured by Gel Doc gel documentation software (Bio-Rad). Fold induction represents the ratios of FSK-treated versus untreated specimens from each cell line.

FIG. 4 shows the effects of a selective inhibitor of PKA pathway H-89 on ARCaP CM-, db cAMP- or FSK-induced hOC and hBSP promoter activities. A, C4-2B cells, transfected with hOC or hBSP promoter-reporter constructs, are treated with various concentrations of H-89 (from 10⁻⁸ M to 10⁻⁶ M) for 2 h, and subsequently exposed to FSK (10⁻⁶ M) for an additional 16 h. H-89 exerted a concentration-dependent inhibition of hOC and hBSP promoter-reporter activities induced by FSK. Fold induction represents the folds of FSK treated reporter activities assayed in the presence or absence of H-89. Levels of significance are calculated: *, p<0.05; **, p<0.005. B, H-89 inhibited ARCaP CM-, db cAMP- or FSK-induced hOC promoter activity, but do not inhibit the promoter-reporter activity assayed under stimulation by PMA. H-89 (10⁻⁶ M) is added to hOC promoter-reporter transiently transfected C4-2B cells for 2 h, then exposed to ARCaP CM (15 μg/ml), db cAMP (10⁻³ M), FSK (10⁻⁶ M), or the PKC pathway activator PMA (10⁻⁶ M) for 16 h. C, H-89 also abolishes the hOC promoter activation induced by C4-2B, DU145, PC3 or MG63 CM. Data are expressed as the mean±S.D. of three independent experiments with duplicate assays. Significant differences are calculated as: *, p<0.05; **, p<0.005.

FIG. 5 shows that ARCaP CM and FSK enhance CREB and CRE binding through cAMP-dependent PKA signaling pathway in selective human prostate cancer but not bone cells. A, C4-2B cells are exposed to ARCaP CM (CM, 15 μg/ml) or FSK (F, 10⁻⁵ M) for 16 h; control cells are exposed to vehicles. Cells are harvested and nuclear extracts (NE) prepared. EMSA is performed by incubating nuclear extracts and the ³²P-labeled CRE probe. Lanes 3 and 5 show ARCaP CM and FSK enhance the formation of CRE-nuclear protein complexes. The complexes are competed off by unlabeled specific CRE-oligo probe (lanes 4 and 6). Lane 9 presents that the Mut6-oligo (the CRE mutant of two-point substitution, see FIG. 2D) do not compete with the nuclear proteins and ³²P—CRE-oligo complexes. H-89 (10⁻⁶ M) blocks both ARCaP CM- and FSK-induced CRE binding to the nuclear factors extracted from C4-2B cells (lanes 7 and 8). B, C4-2B (lanes 1-3) and MG63 (lanes 4-8) cells treated with ARCaP CM (CM, 15 μg/ml) or vehicle for 16 h and nuclear extracts are prepared. Lanes 4 and 5 show no or minimum changes of the binding complex formation when experiments are conducted using nuclear extracts from MG63 cells either exposed to ARCaP CM or not. The arrow indicates the CRE and CREB complexes which are supershifted by adding anti-CREB antibody to the nuclear extracts from C4-2B (lane 2), but not from MG63 cells (lane 7). The specificity of the supershift complex is confirmed by the lack of a supershift band when naive Runx2 antibody is used as a reagent (lanes 3 and 8).

FIG. 6 depicts the proposed cAMP-dependent PKA signaling mechanism underlying the regulation of hOC and hBSP promoter activities in human prostate cancer cells. An unknown soluble factor with a molecular mass of less than 30 kD is proposed to be secreted by human prostate cancer and bone stromal cells. This putative factor may interact with a cell surface receptor in prostate cancer cells and subsequently activate adenylate cyclase (AC), resulting in activation of hOC and hBSP promoter through a PKA signaling pathway. The molecular basis for osteomimicry is proposed as follows: cAMP generated by ligand-receptor interaction promotes PKA activation; the activated PKA is then translocated to the nucleus to induce CREB phosphorylation. The phosphorylated CREB, in turn, interacts with CRE cis-elements in hOC(CRE) and hBSP (CRE1 and CRE2) promoters and activates transcription in human prostate cancer cells.

FIG. 7. A. The endogenous β2M (β2 microglobulin) mRNA expression (RT-PCR) and the β2M protein expression in human prostate cancer cell lines, LNCaP, C4-2B, DU145, PC3 and ARCaP and a human osteosarcoma cell line, MG63. Note despite similar levels of β2M mRNA expression in prostate cancer and bone cells, the secreted form of β2M protein correlates positively with the malignant status of prostate cancer cells. B. β2M stimulates the growth of all human prostate cancer (ARCaP, C4-2B, DU145, and LNCaP cells) but not bone (MG-63) cell lines in culture. * p<0.05. C. β2M over-expression in C4-2B cells markedly increases the endogenous OC and BSP mRNA expression (upper panel). Recombinant β2M protein (0-0.6 mg/ml of β2M) stimulates hOC and hBSP promoter activities and these increased promoter activities can be blocked specifically by anti-b2M antibody (10 mg/ml) but not the isotype control IgG. **, p<0.005 (right panel). D. β2M—but not scramble—siRNA inhibits both cell proliferation and β2M expression of Neo and β2M-overexpressed C4-2B clones. However, scramble β2M-siRNA do not affect these parameters in Neo and b2M clones. **, p<0.005. E. β2M-overexpressed C4-2B cells (C4-2B β2M) grow rapidly in nude mice bones with rapid rise of serum PSA (compared to Neo transfected C4-2B cells), but only small differences are observed with tumor grown in subcutaneous space (N=8-12). Both osteolytic (TRAP+) and osteoblastic lesions are observed in mouse bone (bottom panels). Serum PSA, x-ray, and histopathology are routinely assessed in our studies.

FIG. 8 shows the establishment of overexpression β2M in breast cancer (MCF7), lung cancer (H358) and renal cancer (RCC) cell lines. Different β2M expression levels of MCF7, H358 and RCC clones are assayed by semi-quantitative RT-PCR (top panels). β2M promotes cancer cell proliferation on plastic (middle panels) and in soft agars as revealed by increasing number and size of the colonies (bottom panels). Increased cancer cell proliferation by β2M is β2M concentration-dependent in various human cancer cell lines. An asterisk indicates p<0.05 compared with parental and Neo transfected cells.

FIG. 9. A. Phosphorylation of CREB and its highly homologous transcription factor ATF-1 in C4-2Bneo and C4-2Bβ2M cells as determined by Western blotting. B. Confirmation of phospho-CREB expression in human prostate cancer specimens by immunohistigochemistry staining. C. Expression of VEGF isoforms and the co-receptor neuropilin-1 in C4-2Bneo and C4-2Bβ2M cells by RT-PCR analysis.

FIG. 10 shows non-invasive bioluminescence imaging assessing real-time visualization of prostate cancer metastasis in transgenic mouse models. A. A representative bioluminescence profile in TRAMP-Luc models with an exception of the #7 mouse (column) shows an increase signal at mouse jaw and hind limbs at week 22. B. The prostate tumor and lymph node metastases are confirmed by IHC of SV-40 T antigen. C. Abnormal cellular component (see arrow) is observed on the section of jaw bone derived from #7 mouse by histomorpathological (H&E) analysis.

FIG. 11 shows an in vivo detection of experimental metastasis after intracadiac injection of luciferase gene transfected PC3M (PC3M-Luc) human prostate cancer cells into athymic nude mice. Selected in vivo imaging of a representative mouse with metastasis is shown over time. Micro metastases (arrows) to liver (day 21), adrenal gland and tibia (Day 28) are detected by CCD camera.

FIG. 12 shows liposome encapsulated β2M-siRNA, but not scramble-siRNA, inhibits the growth of pre-established PC3-Luc and C4-2-Luc tumor in athymic nude mice. The anti-tumor effect of siRNA in subcutaneous bone powder tumors (A, B) and intratibial bone tumors (C) is demonstrated by BLI (A, N=5) and serum PSA (B, N=5 and C, N=7-9) over a period of 28 days. ** p<0.005. (D) histomorpathological (H&E) analysis (Magnification: 200×) show massive prostate cancer cell death in β2M-siRNA treated specimens.

FIG. 13 shows an adhesion assay of β2M-siRNA and scramble-siRNA infected C4-2B cells using 96-well plate pre-coated with Col I, LM, FN and Col IV. BSA is used as control (Con). *, p<0.05, **, p<0.005.

FIG. 14 shows a Western blot analysis of parental C4-2B (P), β2M-siRNA (siRNA) and scramble-siRNA (Scramble) retrovirally infected cells. Note AR and PSA expression are abolished by β2M-siRNA in C4-2B cells. EF-1α is used as loading control.

FIG. 15 shows the dot plot for β2M and VEGF between two groups (1 is for bone metastasis group and 2 for tumor confined group).

FIG. 16 depicts the involvement of osteomimicry in driving epithelial to mesenchymal transition (EMT) and EMT-associated gene expression during malignant progression of cancer cells.

FIG. 17 shows an X-Ray photograph depicting the development of osteoblastic/osteolytic mixed tumors in a control mouse versus a β2M knockout SCID mouse.

FIG. 18 shows β2M regulation of VEGF expression and signaling in prostate cancer cells. The β2M-induced activation of cAMP-PKA-CREB pathway facilitates the formation of a dynamic transcriptional complex, recruiting several important transcriptional factors, i.e., CBP/p300, HIF-1, STAT3, AR and SRC-1, to bind the VEGF promoter and activate transcription. Elevated VEGF expression and secretion in turn activates certain downstream signaling in an NP-1-dependent manner. This autocrine loop antagonizes the pro-apoptotic effects of Sema3A/3B, thereby, promoting cancer cell proliferation and metastasis.

FIG. 19 depicts the signaling pathways of the GPCR axis.

FIG. 20 depicts the androgen receptor (AR) signaling pathway.

FIG. 21 shows that the anti-β2-M antibody induces cell death of cancer cells.

FIG. 22 shows the pro-apoptotic activity of anti-β2M antibody in prostate cancer cells.

FIG. 23 shows a physical interaction between β2M and HFE protein.

FIG. 24 shows β2M-blocking agents may induce iron upload and apoptosis. The β2M-blocking agents may include anti-β2M antibody and the β2M-binding domain of HFT protein.

### DETAILED DESCRIPTION

Embodiments of the invention relates to reagents and methods of interfering with osteomimicry of cancer cells. These reagents and methods can be used in cancer prevention and treatments. In accordance with embodiments of the invention, these reagents and methods may target the functions of β2-microglobulins (β2M), which is involved in osteomimicry. In accordance with embodiments of the invention, “osteomimicry-interfering drugs” may include β2M siRNA, β2M antisense, small molecule inhibitors of β2M transcription/translation, anti-β2M antibodies, and β2M-binding domain of HFE protein.

β2M is a small invariable light chain subunit of the class I major histocompatibility complex (MHC, or HLA in humans) presented on the cell membrane of all nucleated cells. When MHC molecules turnover, β2M is shed from the cell membrane into blood. Lymphocytes are the main source of serum free β2M. Serum or urine β2M concentration increases in several malignant diseases, including prostate cancer, myeloma, lung cancer, renal cancer, lymphocytic malignancies, and some inflammatory and autoimmune disorders. Therefore, serum β2M has prognostic values in these diseases.

Interferons (IFNs) can enhance the expression of class I and II MHC molecules. Accordingly, IFNs can increase the formation β2M, which helps to present MHC molecules onto cell membranes, decrease tumor evasiveness and thus enhance host defense mechanisms against tumor growth. IFN alpha is used in diseases like multiple myeloma, where serum β2M measurements can be used to assess tumor burden. Because MHC presentation is associated with host acquired immunity, decreased β2M or lost MHC expression could contribute to tumor cells' evasiveness, as with enhanced engraftment in patients who received bone marrow transplantation.

Increased β2M levels promote growth of prostate cancer, myeloma, and bone and dendritic cells. The mechanisms may involve increased expression of IL 6, 8 and 10 by a number of cancer cell types, bone-like proteins in prostate cancer cells, and critical growth factor receptors, notably type 1 and 2 IGF receptors and EGF receptor, that enhance tumor growth. Previous work on β2M in myeloma revealed that the concentrations of this protein in serum and bone marrow aspirate correlated inversely with patient prognosis.

The inventors of the present invention have identified a novel molecular target, osteomimicry, which confers the ability of prostate cancer cells to mimic the gene expression and behaviors of osteoblasts, thus allowing prostate cancer cells to adhere to bone cells and grow and survive in bone. Osteomimetic prostate cancer cells not only express highly restricted bone-like proteins, such as osteocalcin (OC), bone sialoprotein (BSP), osteopontin (OPN), and receptor activator of NFκB ligand (RANKL), but they also are capable of forming mineralized bone under certain cell culture conditions. The observations that bone matrix proteins are highly expressed in both localized and metastatic prostate cancers, but not in normal prostate, further support the osteomimetic nature of the prostate cancer cells.

Osteomimicry is defined as the ability of cells, non-malignant cells (e.g., benign prostate hyperplasia and fibromuscluar stromal cells around the blood vessels) or cancer cells, to grow and mimic the gene expression and behaviors of bone cells. Inventors of the present invention have found osteomimicry allows the cancer cells to grow, survive and invade in the bone microenvironment. Osteomimicry may also regulate host immunity and other immune status.

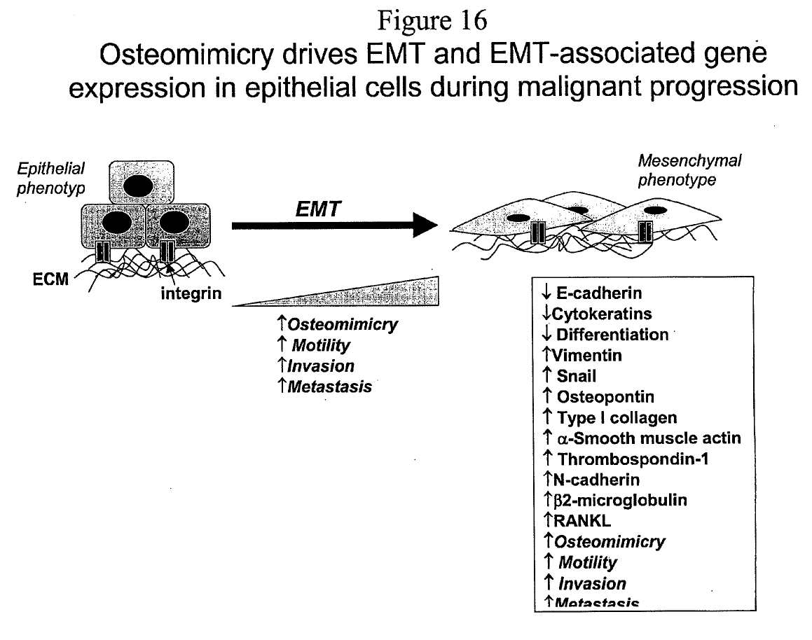

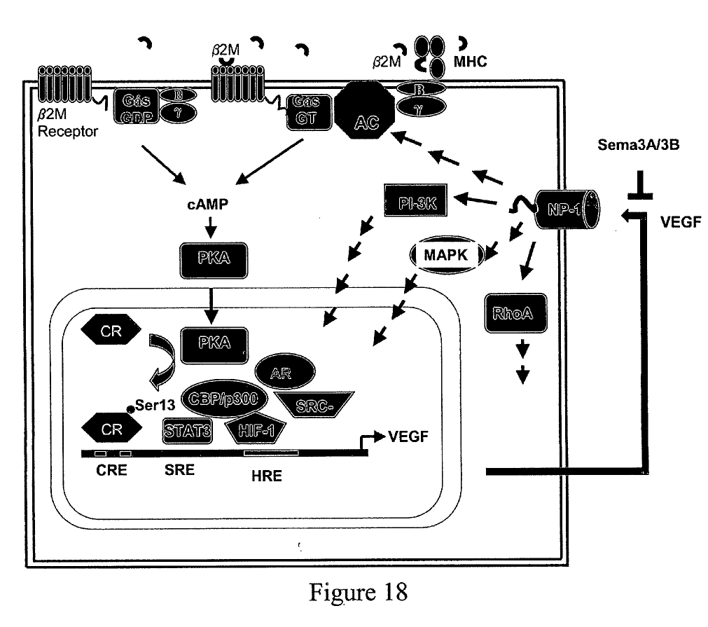

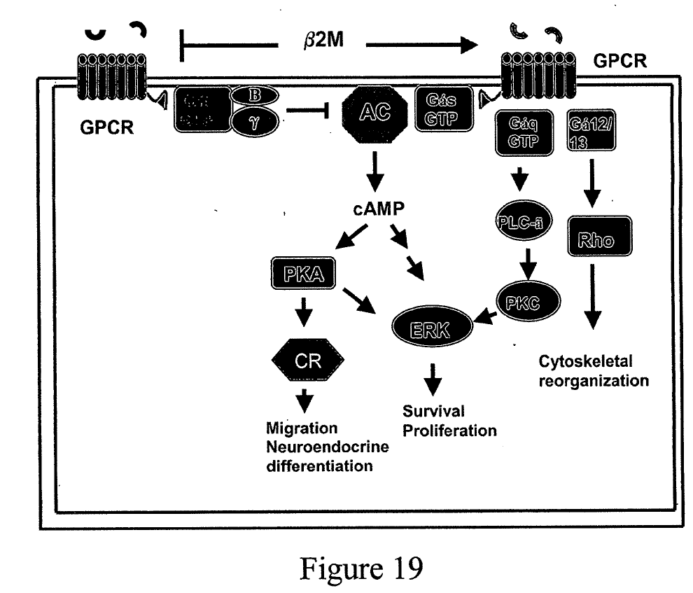

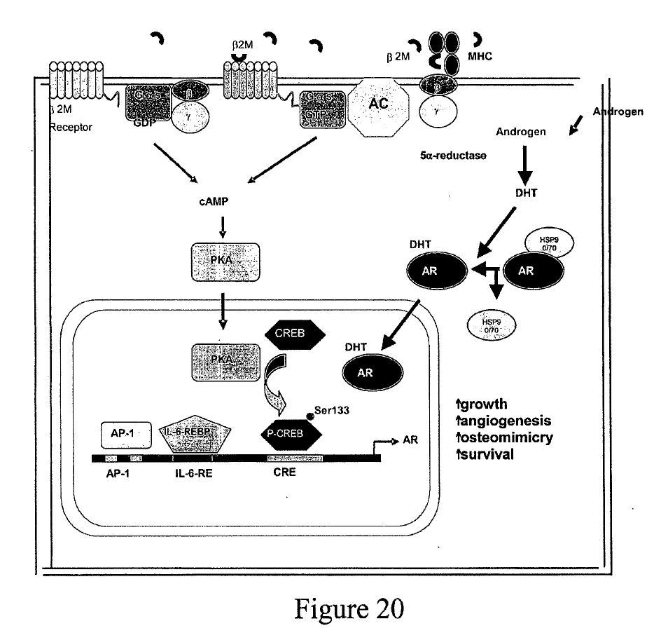

Osteomimicry is controlled by: (1) the cAMR/PKA/CREB pathway which is tied to GPCR-mediated downstream signaling (FIG. 19), AR axis (FIG. 20), VEGF axis (FIG. 18), EMT, integrin-ECM signaling (FIG. 16); and (2) the Runx2/cbfal signaling pathway. As a result, osteomimicry controls the ability of cancer cells and non-cancerous cells in cancer microenvironment to grow, undergo apoptosis, gain survival advantages, invade, migrate, metastasize, and/or differentiate. Consistently, osteomimicry is responsible for the synthesis, secretion and deposition of the bone like proteins: OC, OPN, ON, BSP and RANKL by benign and cancer cells.

Table 1 shows that osteomimicry may affect the fate of benign and cancer cells by regulating a series of genes related to the control of cell growth, cell death, oxidative stress, cell differentiation and cell cycle progression.

Osteomimicry occurs in normal cells which allows them to calcify and mineralize, providing a foundation for the development of BPH and atherosclerotic plaques. Osteomimicry affects the presentation of MHC class-1 antigen in normal cells and affects the immunity and immune status of the host.

TABLE 1

Function

b2M Target Genes (increased)

| | |
|---|---|
| b2-Adrenergic receptor | cell growth |
| VEGF | cell growth, cell cycle |
| STAT3 | cell mobility |
| Glutathione peroxiase | oxidative stress |
| PDGF b peptide | |
| ADAM17 | |
| IL-8 receptor b | cell growth, mobility |
| b2M | cell growth, survival |
| IGF2 | cell growth |
| PSA | prostate cancer |
| | progression |
| Tumor protein D52 | |
| CREB-like2 | cell growth, survival |
| STAT1 | apoptosis |
| b-Catenin | cell adhesion |
| G protein-coupled | cell growth, survival |
| receptor 56 | |
| IGFBP3 | cell growth |

b2M Target Genes (decreased)

| | |
|---|---|
| IGF2R | cell growth |
| Heat shock 70 kDa protein 4 | |
| ADAM15 | cell adhsion |
| Vimentin | EMT marker |
| IGFBP2 | cell growth |
| IGF1 | cell growth |
| Phosphodiesterase 3A | |

Osteomimicry has dual functions: (1) Overexpression of osteomimicry genes in benign or cancer cells may increase growth survival and decrease apoptosis. Therefore, antagonizing osteomimicry (e.g., using osteomimetic interfering drugs) may inhibit cell growth and increase apoptosis. (2) Overexpression of these genes in normal host cells may enhance host immunity, leading to decreased efficiency of bone marrow and stem cell engraftments. Therefore, osteomimetic interfering drugs may be used to suppress host immunity and increase the efficiency of cell engraftments.

Drugs that interfere with osteomimicry can block cancer progression by causing cell death, abrogating neovascular endothelial sprouting and ingrowth of endothelium into the invasive tumor, preventing EMT (embryonic-mesenchymal transition), inhibiting attachment of cancer cell to selected ECM and attenuating cancer cell survival. These drugs are also expected to decrease calcification and mineralization of normal benign cells and cause apoptotic death of BPH and fibromuscular smooth muscle cells

Drugs that interfere with osteomimicry include those known to interfere with the Runx2 signaling pathway. Osteomimitic interfering drugs may be used in combination with other cytotoxic drugs to enhance the therapeutic affect. For example, osteomimitic interfering drugs may be used either alone or in combination to inhibit growth and metastasis of cancers including, but not limited to, prostate, breast, multiple myeloma, renal, lung, brain, thyroid, colon, and osteosarcoma. In addition, the osteomimitic interfering drugs may inhibit abnormal growth of benign cells including, but not limited to, smooth muscle cells and fibroblasts related to mesenchymal lineage in the benign state such as BPH and atherosclerosis and host immunity during bone marrow and stem cell transfer.

Bone matrix proteins and signal transduction molecules related to osteomimicry (involved in AR axis, VEGF axis, GPCR axis, cAMP/PKA/CREB axis, and Runx2 signaling pathways) are present in biologic fluids or tissues may serve as biomarkers to predict cancer, bone and visceral organ metastases, and the phenotypes of cancers.

For example, osteomimicry may be determined by a soluble factor, β2M or β2M-like protein or peptide. As noted above, β2M is part of an MHC complex. However, β2M may be secreted by cancerous or normal cells to activate downstream target genes such as bone matrix proteins and signal molecules involved in AR axis, VEGF axis, GPCR axis, cAMP/PKA/CREB axis, and Runx2 signaling pathways through transcriptional activation of, but not limited to, CREB (Table 1).

Osteomimicry may be assayed using a cell transfected with a construct hacing an osteomimicry target gene (such as human osteocalcin (OC) gene) promoter and a reporter (e.g., luciferase), either alone or in combination with a host of other osteomimicry target gene promoter reporter constructs. The extent of osteomimicry may be correlated with the degree of activation of these reporter constructs. Thus, osteomimicry interfering drugs may be assayed by assessing the expression of the report gene products. The target cell may include, but not limited to, prostate, breast, multiple myeloma, renal, lung, brain, thyroid, colon, and osteosarcoma. Likewise, the effect of osteomimetic interfering drugs on abnormal growth of the benign cell may be assayed. The benign cell may include, but not limited to, smooth muscle cells and fibroblasts related to mesenchymal lineage in the benign state, such as BPH and atherosclerosis, and host immunity during bone marrow and stem cell transfer.

Osteomimitic interfering drugs may include, but not limited to, small molecules, antibodies, nucleic acids, and naturally occurring pharmaceuticals. These drugs may be assayed to determine their effect on osteomimicry by assessing their ability to interfere promoter reporter activity, cell growth, cell survival, apoptosis, cell invasion, cell migration, and cell spreading.

Osteomimitic interfering drugs may include, but not limited to, nucleotide sequences or their fragments that recognize the promoter regions regulating downstream target from osteomimicry. These targets may include, but not limited to, AR axis, VEGF axis, GPCR axis, cAMP/PKA/CREB axis, and Runx2 signaling pathways and genes described in Table 1.

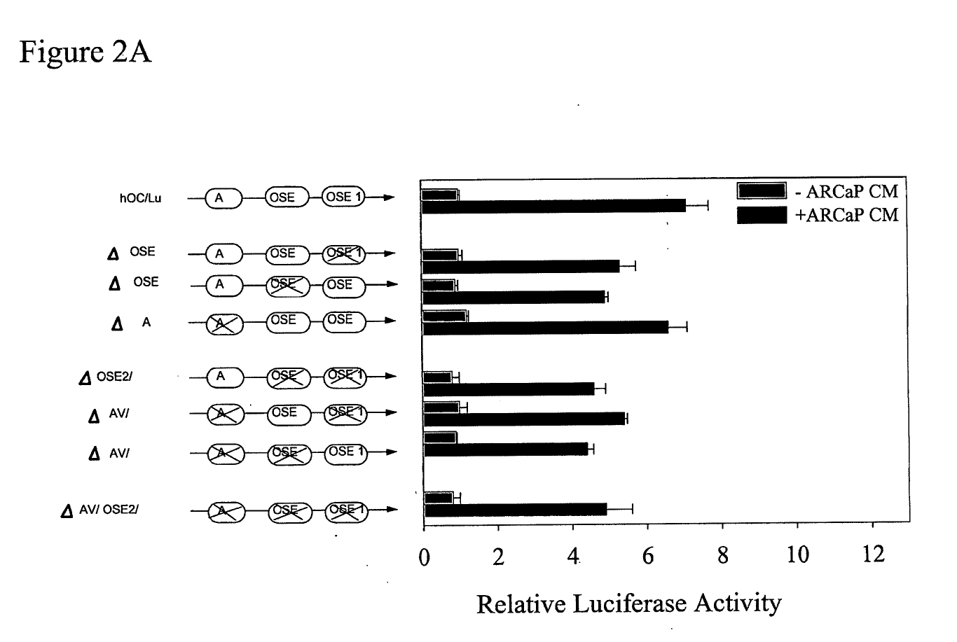

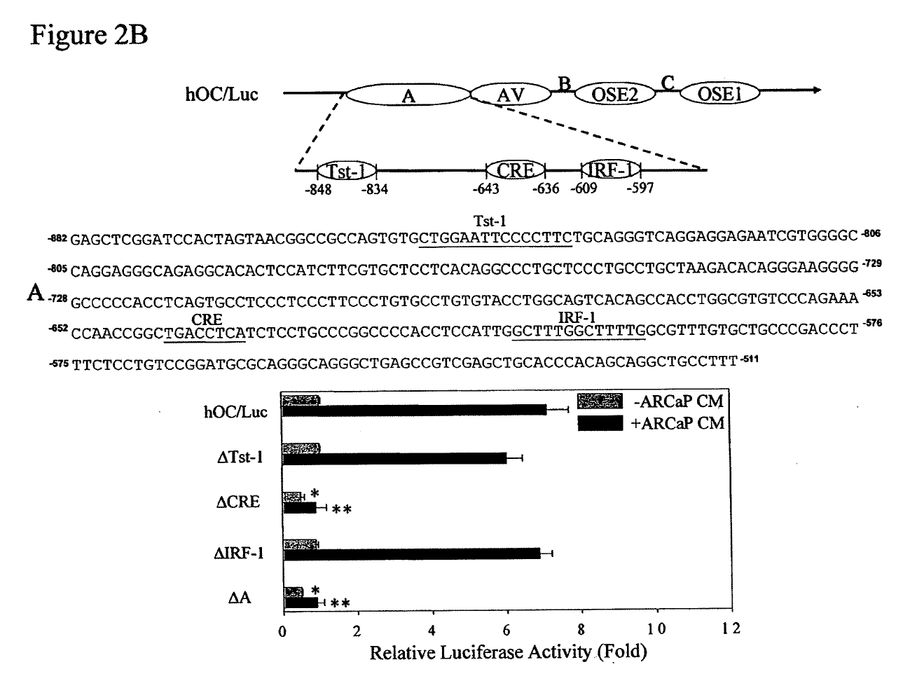

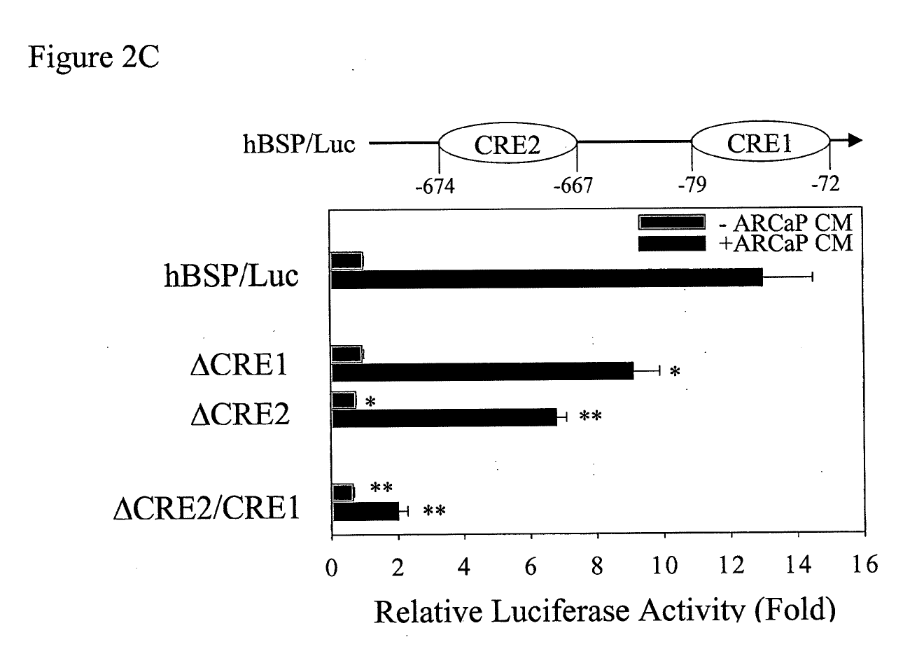

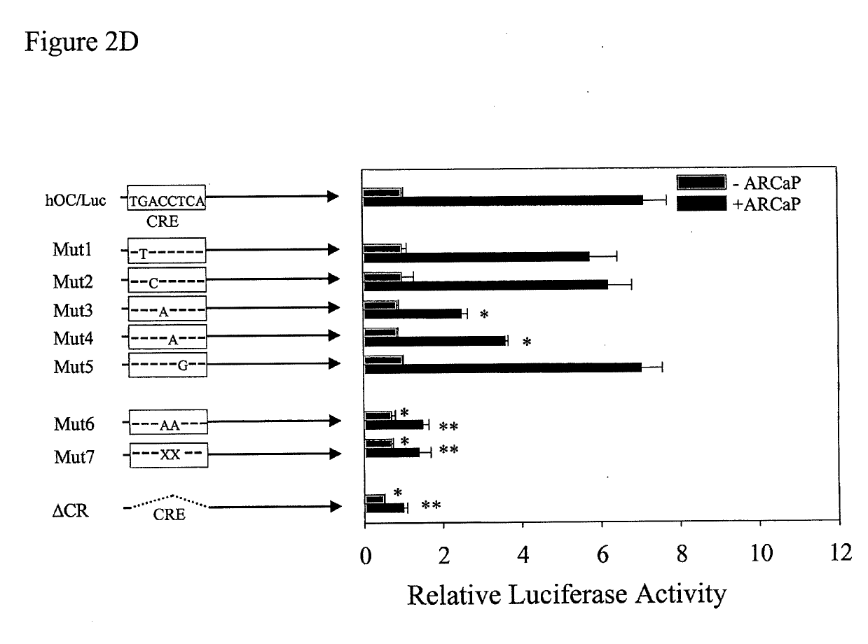

Osteomimitic interfering drugs may include, but not limited to, analogs of small molecules that interfere with the AR axis, GPCR axis, VEGF axis, and PKA/CREB axis. For example, osteomimitic interfering drugs may include those that interfere with PKA/CREB signal activation. Specifically, these drugs may target the regions of the cis-acting elements, between −643 and −636 (CRE), in the hOC promoter (FIG. 2). This region may be responsible for the cAMP-mediated transcriptional regulation in human prostate cancer cells. Another example of osteomimitic interfering drugs may interfere with the activation of PKA/CREB signal pathway. The drugs may specifically target the CRE elements located in the hBSP promoter, e.g., −79 to −72 (CRE1) and −674 to −667 (CRE2) (FIG. 2). The CRE elements may also be activated by cAMP mimetic and yet unidentified growth factor(s) present in human prostate cancer cells, conditioned media (CM) of prostate cancer, and bone stromal cells.

Osteomimitic interfering drugs may include antibodies that interfere with osteomimicry. These antibody drugs may include, but not limited to, binders to and/or interfering molecules composed of a protein, peptide, nucleic acid, and radioactive/cytotoxic derivatives having the ability to interfere with the osteomimicry related downstream signaling.

### Example 1

EXAMPLE 1 shows that osteomimicry in prostate cancer cells may be maintained by the activation of G-protein coupled Protein Kinase A (PKA) signaling pathway, which is mediated by a cAMP responsive element binding protein (CREB). By targeting this osteomimetic processes, osteomimetic interfering drugs may inhibit prostate cancer cell growth, induce apoptosis in tumor cell in vitro and in vivo xenograft models. Therefore, specifically targeting osteomimicry either alone or in combination with chemotherapy, may inhibit prostate, breast, lung and renal cancer cell growth and survival in bone, thus, leading to increased survival of cancer patients with bone metastases.

(1) Expression of OC and BSP Proteins by Clinical Prostate Cancer Tissue Specimens, and Conditioned Media (CM) Collected from Human Prostate Cancer and Bone Stromal Cells Stimulated Human OC (hOC) and Human BSP (hBSP) Promoter Activities and The Steady-State Levels of Endogenous OC and BSP mRNA Expression in Human Prostate Cancer Cell Lines.

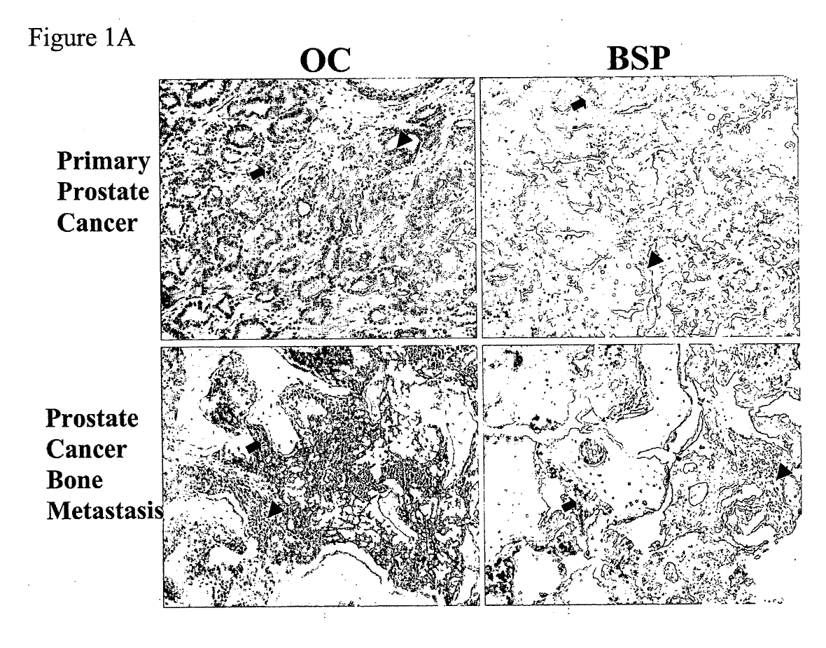

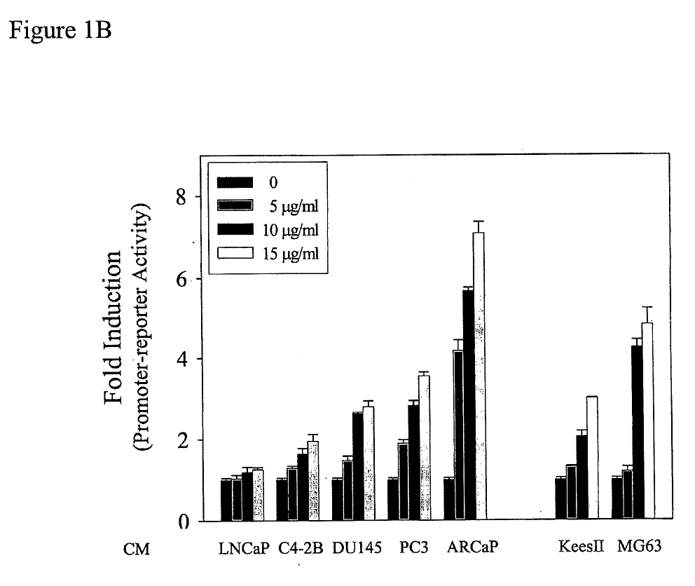

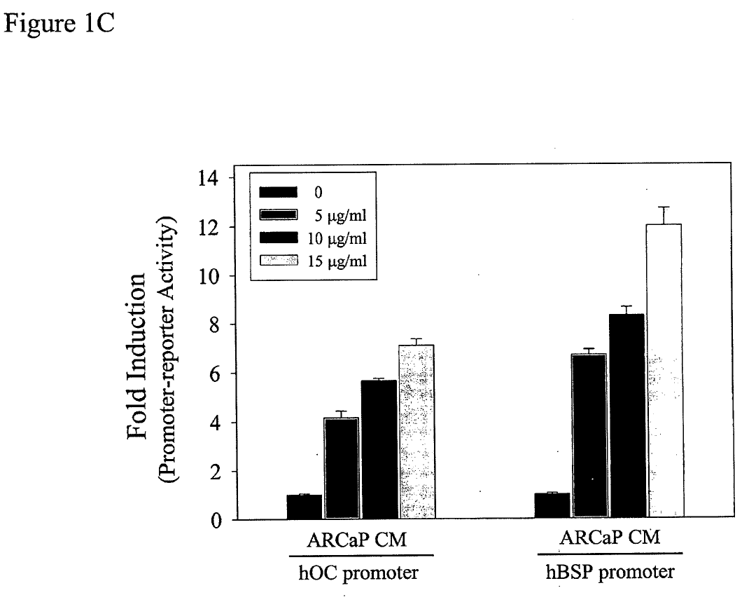

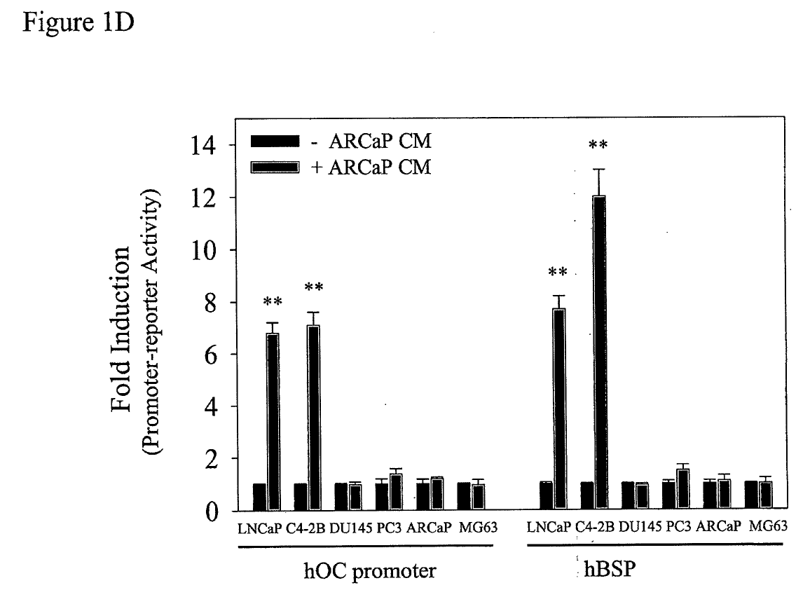

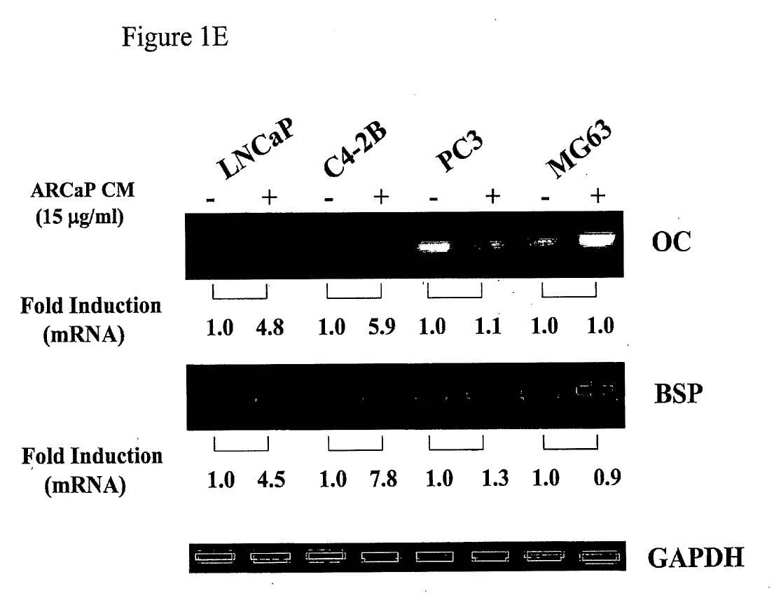

OC protein is prevalently expressed by both primary (85% positively stained) and metastatic (both lymph node and bone were 100% positively stained) human prostate cancer specimens. Likewise, BSP protein is also expressed preferentially by malignant (89-100%) primary prostate cancer tissues. One common feature of OC and BSP immunostaining in human prostate cancer tissues is the marked heterogeneities among prostate cancer cells in primary and bone metastatic specimens. Some cells stain strongly for OC and BSP proteins (FIG. 1A, bold arrows) and others seem to be lightly stained or not stained at all (FIG. 1A, arrow heads). The differential staining may reflect either intrinsic genetic variations among the prostate cancer cells or be an epiphenomenon of prostate cancer cell interaction with the microenvironment. To define the role of extrinsic factor(s) secreted by prostate cancer or bone cells can mediate OC and BSP promoter activities and the steady-state levels of endogenous mRNA, the regulation of OC and BSP expression in human prostate cancer and bone cells is monitored.

The effects of CM (conditioned media) harvested from either prostate cancer or bone stromal cells on the expression of OC and BSP in an androgen-independent C4-2B prostate cancer cell line derived from of LNCaP are assessed. Briefly, a luciferase reporter construct with a 0.8-kb hOC promoter or a 1.5-kb hBSP promoter is transfected into C4-2B cells, and the transfected cells are exposed to serum-free CM collected from various human prostate cancer cell lines with a gradient of malignant potentials [from LNCaP (the least malignant), C4-2B, DU145, and PC3 (cells with intermediate levels of aggressiveness) to ARCaP (the most malignant)], a normal non-malignant osteoblast KeesII cell line or a malignant osteosarcoma MG63 cell line.

FIG. 1B shows that CM stimulates hOC promoter activity in a concentration-dependent manner (from 0 to 15 μg/ml of total proteins in CM). CM from LNCaP cells maximally stimulate hOC promoter activity by only 1.2±0.1-fold, whereas CM collected from the most aggressive ARCaP prostate cancer cell line maximally enhances the highest hOC promoter activity at 7.1±0.3-fold. CM from other cell lines, C4-2B, DU145, PC3, KeesII and MG63, induces hOC promoter activity at intermediate levels (from 2.0±0.2 to 5.1±0.4-fold). These data suggest that the extent of stimulation of hOC promoter activity by CM correlates positively with the aggressiveness of the prostate cancer. In parallel with the induction of hOC promoter activity, ARCaP CM also may up-regulate the hBSP promoter activity as much as 12-fold in a concentration-dependent manner in C4-2B cells (FIG. 1C).

To determine whether ARCaP CM is capable of stimulating hOC and hBSP promoter activities in other human prostate cancer and bone stromal cell lines, the activity of these promoters are tested in LNCaP, DU145, PC3, ARCaP and MG63 cells. FIG. 1D shows that both hOC and hBSP promoter activities are elevated by ARCaP CM in LNCaP and C4-2B, but not in DU145, PC3, ARCaP and MG63 cell lines.

FIG. 1E shows that treating LNCaP and C4-2B cells with ARCaP CM (15 μg/ml) for 12 h results in an increase of the steady-state levels of endogenous OC and BSP mRNA expression by 4.8 and 5.9-fold, and 4.5 and 7.8-fold (GAPDH as an internal control), respectively, as determined by semi-quantitative RT-PCR. In cells that have high basal levels of OC and BSP mRNA, such as PC3 and MG63 cells, ARCaP CM does not further enhance OC and BSP mRNA expression (0.9 to 1.3-fold induction, respectively). Similar to that observed in the promoter activity, the steady-state levels of endogenous OC and BSP mRNA may not increase in DU145 and ARCaP cell lines treated with ARCaP CM.

(2) The cAMP-Responsive Element (CRE) May Be Responsible for Regulation of CM-Mediated hOC and hBSP Promoter Activities.

It is known that three cis-acting elements may be critical for the regulation of hOC promoter activity, i.e., OSE1, OSE2, and AP-1/VDRE (AV). FIG. 2A shows that among the single deletion constructs, ΔAV may not affect ARCaP CM-induced hOC promoter luciferase activity. In comparison, a slight decrease of hOC promoter activity is observed upon the deletion of OSE1 or OSE2. No further decrease in hOC promoter-luciferase activity induced by ARCaP CM is noted by deleting additional cis-elements including the complete deletion of all three critical hOC regulatory elements, ΔAV, ΔOSE2 and ΔOSE1. These data suggest that regions outside of OSE1, OSE2 and AV may be responsible for hOC promoter activation by ARCaP CM.

To address this question, three additional constructs with regions outside of the OSE1, OSE2 or AV element systematically deleted are generated. i.e., ΔA (upstream of the AV element, 374 bp, FIG. 2B), ΔB (between AV and OSE2 site, 327 bp), and ΔC (between OSE2 and OSE1 site, 99 bp). FIG. 2B shows a dramatic decrease in hOC promoter activity only when region A is deleted. Minimal loss of ARCaP CM-induced hOC promoter luciferase activity is detected with deletion of region B or C.

To identify the specific cis-DNA element within region A responsible for ARCaP CM-induced hOC promoter activity, the site-specifically deleted regions of A, ΔTst-1 (POU-factor Tst-1/Oct-6, −848 to −834), ACRE (cAMP-responsive element, −643 to −636) and ΔIRF-1 (interferon regulatory factor-1, −609 to −597) in C4-2B cells are tested with and without ARCaP CM. FIG. 2B shows that only the ACRE construct exhibits a marked decrease in ARCaP CM-induced hOC promoter luciferase activity. This result suggests that cAMP mediates downstream signaling through CRE by regulating ARCaP CM-induced hOC promoter activity.

There are two putative CRE sites, CRE1 (−79 to −72) and CRE2 (−674 to −667), located in the hBSP promoter. A hBSP promoter with CRE deletion is generated. FIG. 2C shows that the hBSP promoter luciferase activity decreases partially in the deletion mutants with deletion of CRE1 or CRE2 (designated as ΔCRE1 and ΔCRE2). However, the ARCaP CM-induced activation of hBSP promoter is markedly reduced in a mutant with both sites deleted, ΔCRE2/CRE1. This result shows that CREs may be important for the activation of hBSP promoter activity by ARCaP CM.

To delineate the specific nucleotide(s) within the CRE of hOC promoter responsible for ARCaP CM-regulated promoter activity, the ARCaP CM-induced hOC promoter activity are examined in C4-2B cells using CRE point mutants. FIG. 2D shows that Mut3 (−640 C→A) and Mut4 (−639 C→A) greatly diminish the ARCaP CM-activated hOC promoter activity. Other point mutations, −642 G→T (Mut1), −641 A→C (Mut2) and −638 T→G (Mut5), have little effect on the CM-mediated hOC promoter activity. Consistent with these results, double-base mutations at −640 and −639 CC→AA (Mut6) and deletion at this same region, CC→XX (Mut7), greatly reduce ARCaP CM-induced hOC promoter activity. It is likely that similar mutations in CRE1 and CRE2 may result in disruption of hBSP promoter activity when assayed in prostate cancer cells. Together, the results show that two nucleotides, −640 (C) and −639 (C) within the CRE cis-element of hOC promoter cooperate to activate the hOC promoter in response to ARCaP CM.

(3) The cAMP-Dependent PKA Signaling Pathway May Be Essential for Mediating ARCaP CM-Activated OC and BSP Gene Expression in Human Prostate Cancer Cells.

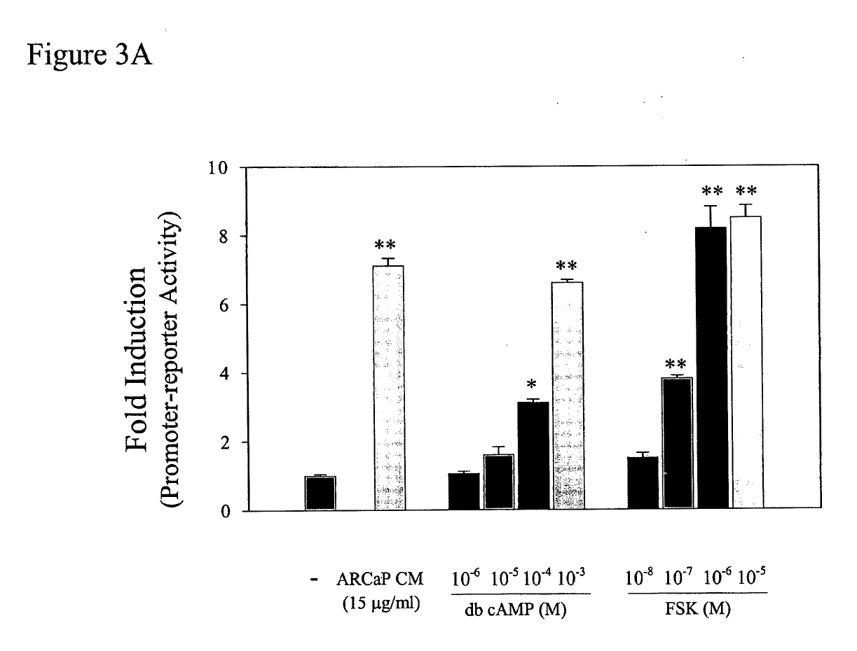

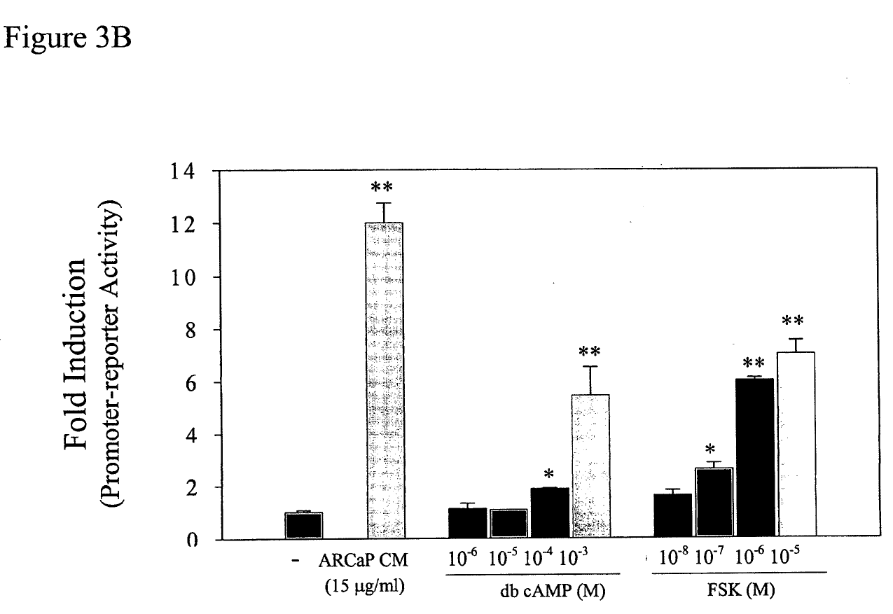

To determine whether the ARCaP CM-mediated activation of hOC and hBSP promoter is mediated through an activation of the PKA signaling pathway, the effects of PKA pathway activators, e.g., dibutyryl cAMP (db cAMP) and forskolin (FSK), on hOC and hBSP promoter activities are monitored. The PKA pathway activators, db cAMP (10⁻⁶ to 10⁻³ M) and FSK (10⁻⁸ to 10⁻⁵ M) stimulate hOC (FIG. 3A) and hBSP (FIG. 3B) promoter activities in a ligand concentration-dependent manner in C4-2B cells. These results are confirmed by testing the endogenous levels of OC and BSP mRNA treated with a PKA activator, FSK. In particular, FIG. 3C shows that FSK treatment (10⁻⁶ M) increases the mRNA expression of OC, and BSP in LNCaP and C4-2B, but not in PC3 and MG63. The steady-state levels of OC mRNA are elevated by 5.2 and 7.8-fold, whereas the levels of BSP mRNA are increased by 3.2 and 5.4-fold by FSK in LNCaP and C4-2B cells, respectively. This result is consistent with the effect of ARCaP CM on endogenous OC and BSP mRNA expression (FIG. 1E), thus, further supporting PKA plays a major role in the downstream signaling pathways that regulate soluble factor-mediated OC and BSP gene expression in LNCaP and C4-2B human prostate cancer cells.

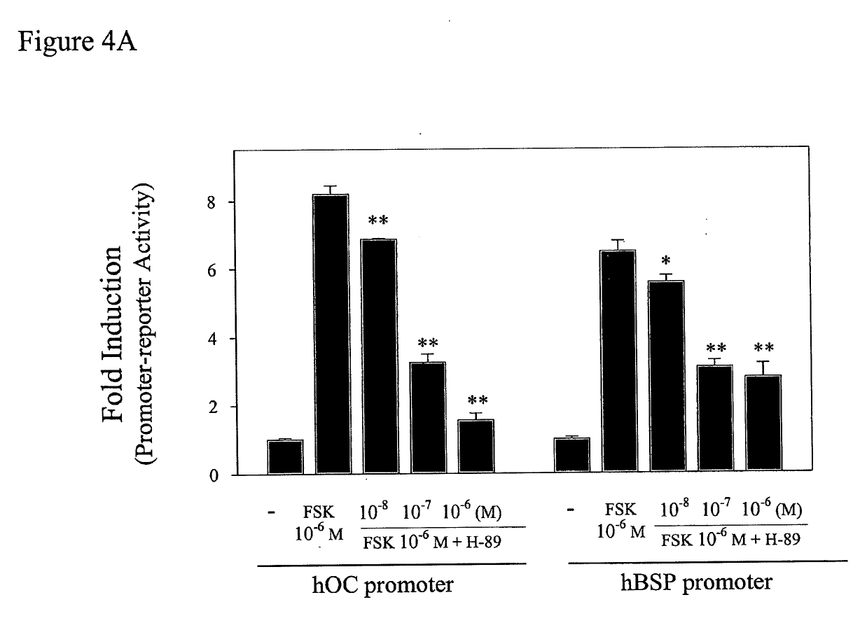

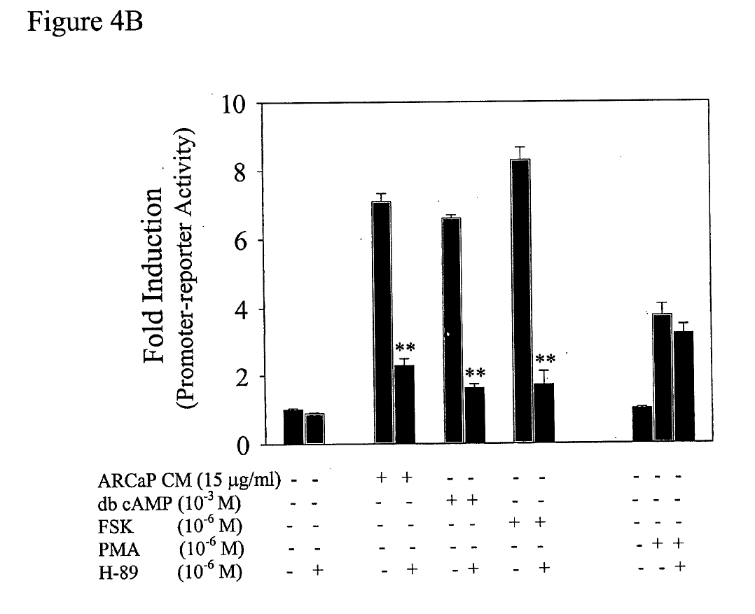

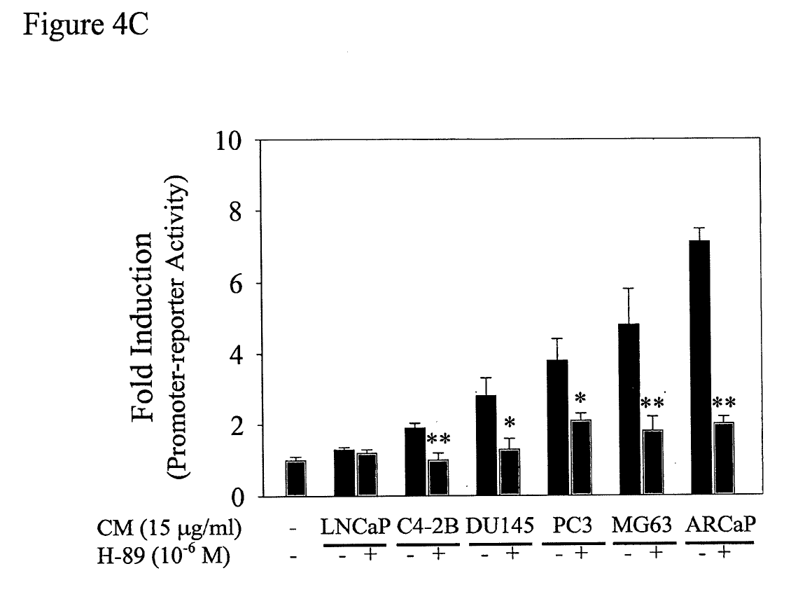

FIG. 4A shows that FSK-stimulated hOC and hBSP promoter activities may be inhibited by H-89 (10⁻⁸ to 10⁻⁶ M) in a concentration-dependent manner. Consistent with this observation, H-89 also inhibits ARCaP CM- and db cAMP-mediated activation of hOC promoter activity in prostate cancer cells (FIG. 4B). Further, PMA, an activator of the PKC pathway, can induce hOC promoter activity to a lesser extent and such activation can be not blocked by H-89 (FIG. 4B). Consistently, H-89 also inhibits the induction of endogenous OC and BSP mRNA expression by ARCaP CM or FSK in LNCaP and C4-2B cells.

FIG. 4C shows that H-89 (10⁻⁶ M) abolish the CM-induced hOC promoter activity in all of CM isolated from prostate cancer and bone stromal cell lines. These data suggest that the soluble factor(s) secreted from prostate cancer or bone CM may activate the gene expression of bone-specific OC and BSP in human prostate cancer cell lines through the cAMP-mediated PKA signaling pathway.

(4) Evidence in Support of Nuclear CRE-Binding Protein (CREB) and Cis-Acting Element, CRE, in the Regulation of Bone-Specific Gene Expression in Human Prostate Cancer Cells: Electrophoretic Mobility Shift Assay (EMSA).

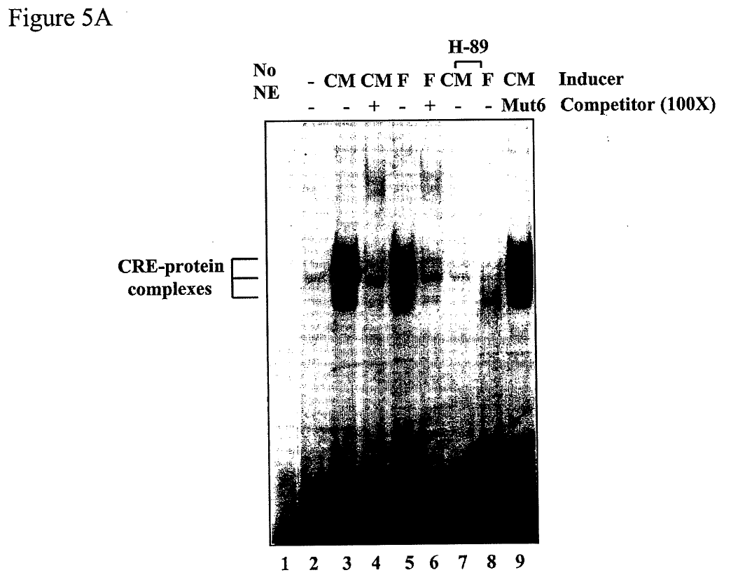

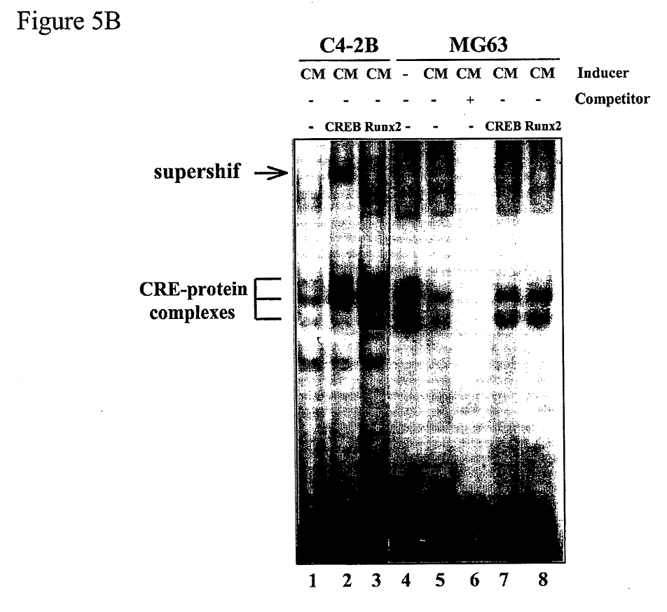

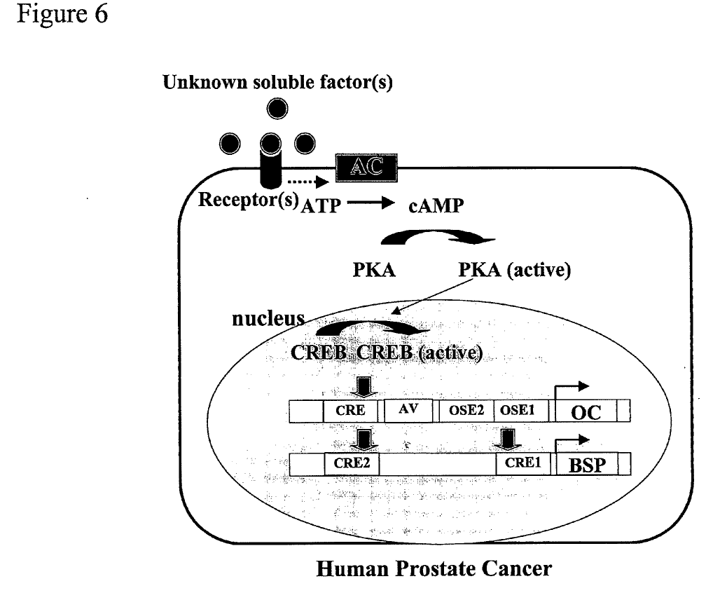

EMSA is employed to establish a downstream link between the cAMP-dependent PKA signaling pathway and hOC and hBSP promoter activation in prostate cancer cells. Briefly, a ³²P-labeled oligonucleotide CRE probe and nuclear factors isolated from C4-2B cells (an ARCaP CM-positive responder) and MG63 cells (an ARCaP CM-negative responder) treated with ARCaP CM (15 μg/ml) or FSK (10⁻⁵ M) are present in a binding reaction and incubated for 16 h. Cells treated with empty vehicle are used as controls. Nuclear factors extracted from either ARCaP CM (CM) or FSK (F) treated C4-2B cells strongly enhance the specific CRE-nuclear protein complex formation (FIG. 5A, lanes 3 and 5) in comparison to cells exposed to control media (FIG. 5A, lane 2). These DNA-protein complexes can be competed off by unlabeled specific CRE-oligo probe (lanes 4 and 6). However, no competition is observed with a mutant form of CRE-oligo probe, the Mut6-oligo (two-point substitution, see FIG. 2D) (lane 9). Consistently, H-89 can abolish both ARCaP CM- and FSK-induced CRE binding to the nuclear proteins isolated from C4-2B cells (lanes 7 and 8). In contrast, nuclear extracts isolated from MG63 cells formed a low but detectable basal level of complexes with ³²P-labeled-CRE probe before and after treatment with ARCaP CM (FIG. 5B, lanes 4 and 5). These complexes could be competed off by unlabeled-CRE probe (lane 6), but fail to be supershifted by anti-CREB antibody (lane 7). As a positive control, nuclear extracts from C4-2B cells treated with ARCaP CM bind to CRE and these CRE-nuclear protein complexes can be supershifted by anti-CREB antibody (lane 2), but not by anti-Runx2 antibody in both cases (lanes 3 and 8). These data demonstrate that the trans-acting factor CREB may play a critical role in the ARCaP CM-regulated bone-specific gene transcription through the cAMP/PKA pathway in human prostate cancer, but not in bone stromal cells.

β2M is a key soluble factor secreted by cancer cells as well as cells in the cancer microenvironment. β2M is a critical autocrine and paracrine growth factor to maintain cancer cells' ability to synthesize and deposit bone-like proteins such as OC and BSP. β2M may also stimulate the growth and survival of cancer cells by activating vascular endothelial growth factor (VEGF) and androgen receptor (AR) signaling pathways resulting in resistance to hormone withdrawal and chemotherapy/radiation therapy. Consistent with this idea, β2M-overexpressing prostate, breast, lung and renal cancer cells may have increased growth rate in both anchorage-dependent and -independent manner. EXAMPLE 2 shows that β2M-overexpressing prostate cancer cells, when introduced into mouse femur, causes rapid tumor growth in bone with elevated serum PSA. This result indicates a direct growth-promoting effect of β2M on prostate cancer bone metastasis. Further, immunohistochemical (IHC) studies of human prostate cancer primary and/or bone metastatic specimens show overexpression of β2M, and its target genes, e.g., OC, BSP, and OPN, are associated with increased malignant status of prostate and breast cancers.

### Example 2

### Evidence for the Role of β2M as a Novel Signaling and Mitogenic Factor Supporting the Growth of Human Prostate Cancer Cells

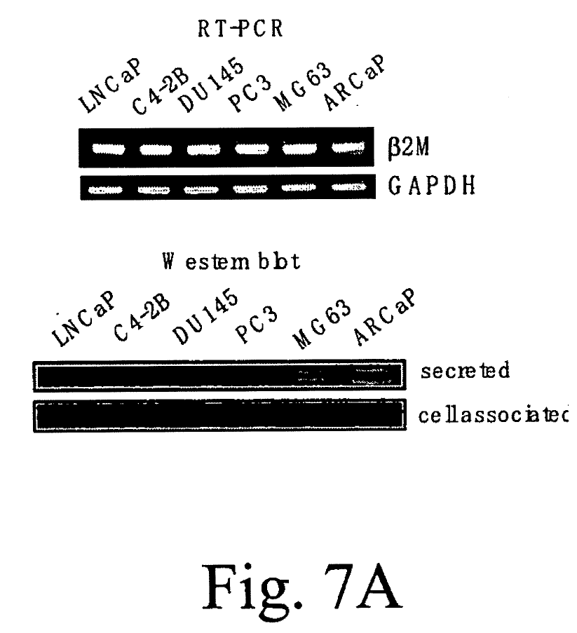

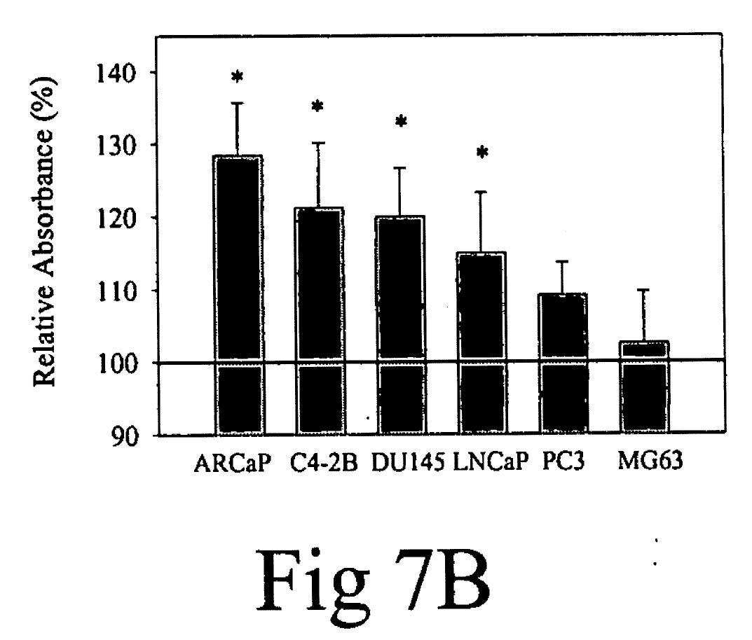

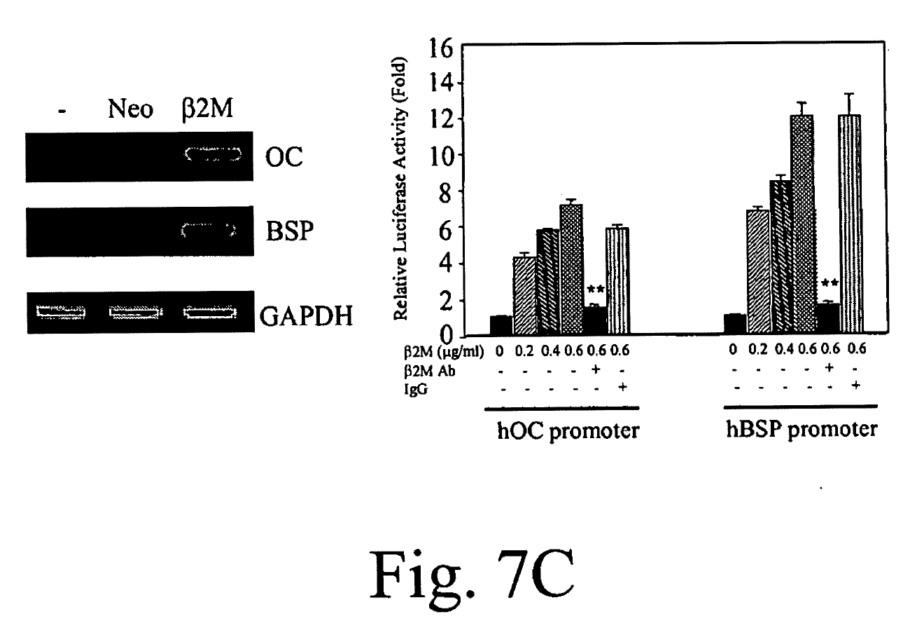

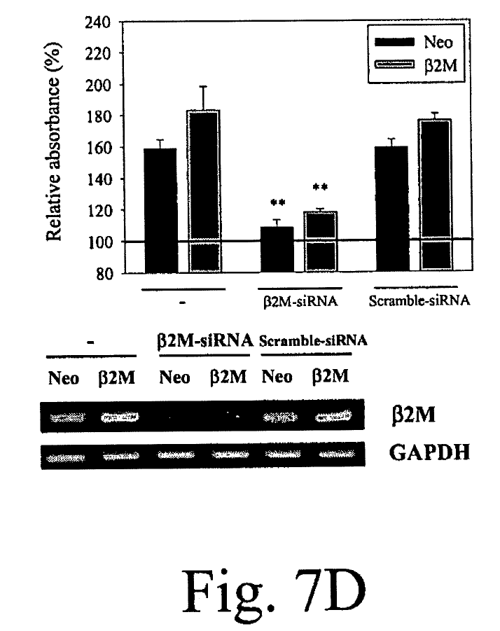

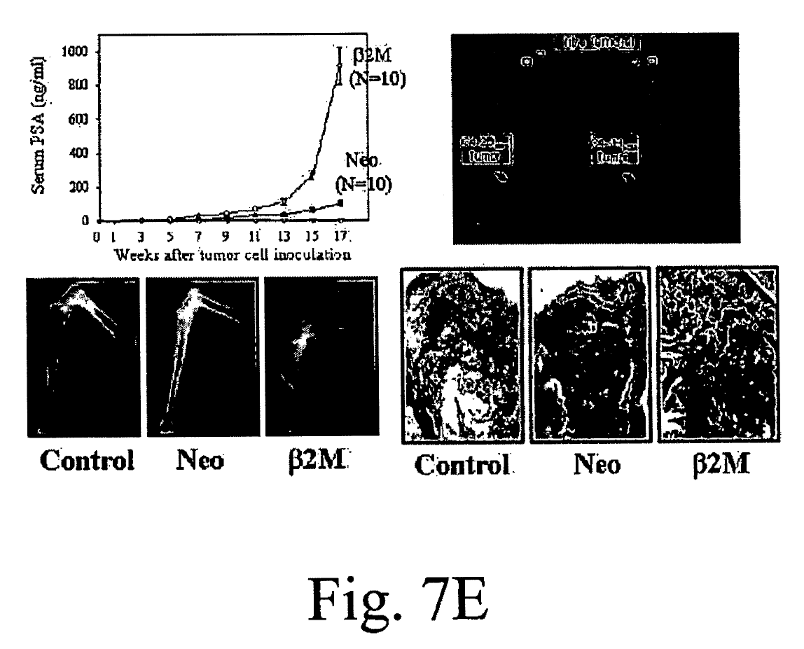

Using ammonium sulfate precipitation, gel filtration, ion-exchange HPLC, and N-terminal amino acid sequencing, a soluble protein factor having a molecular weight of 11.8 kD is isolated. This protein has complete sequence identity with a known protein found in myeloma, β2-microglobulin (β2M), and may confer osteomimicry on prostate cancer cells. Although the steady-state levels of β2M mRNA are similar among prostate cancer cell lines, the secreted β2M protein levels correlate well with the aggressiveness of prostate cancer cell lines in mice with LNCAP as the least aggressive and ARCaP as the most aggressive human prostate cancer cell line (FIG. 7A). Besides being a signaling molecule for osteomimicry, β2M may stimulate the growth of the human prostate cancer cell lines (FIG. 7B). The C4-2B cell line stably transfected with β2M (C4-2Bβ2M) show increased levels of endogenous OC/BSP expression as compared with that in the neo-transfected control clones (C4-2BNeo). FIG. 7C shows that recombinant β2M protein stimulates OC and BSP promoter activity. Importantly, the β2M-mediated induction may be selectively antagonized by anti-β2M antibody. The β2M-mediated increases in prostate cancer cell growth in vitro can also be antagonized by the administration of anti-β2M antibody, but not by the control anti-CREB antibody. Likewise, FIG. 7D shows that β2M siRNA, but not the control scramble-siRNA, inhibits the prostate cancer cell growth in vitro. These results suggest that β2M may be a potent mitogen. Consistently, the β2M-overexpressing C4-2B tumor cells (C4-2Bβ2M) when inoculated in mouse bone, but not subcutaneously, grow rapidly in hosts with greatly elevated serum PSA and mixed osteoblastic and osteolytic lesions (FIG. 7E). These results further suggest that the β2M-induced prostate tumor growth may be mediated by bone cells, due in part to the rich bone microenvironment resulting from increased bone turnover in the presence of prostate cancer cells. There is also 2-fold increase of prostate cancer growth when C4-2Bβ2M cells are injected subcutaneously in nude mice as compared to that of the control C4-2BNeo cells. Tumors isolated from the C4-2Bβ2M are more angiogenic and less necrotic as compared with that of the C4-2BNeo. These results reveal an additional role of β2M as a mitogen for prostate cancer cell growth in vitro and in vivo, and particularly in bone.

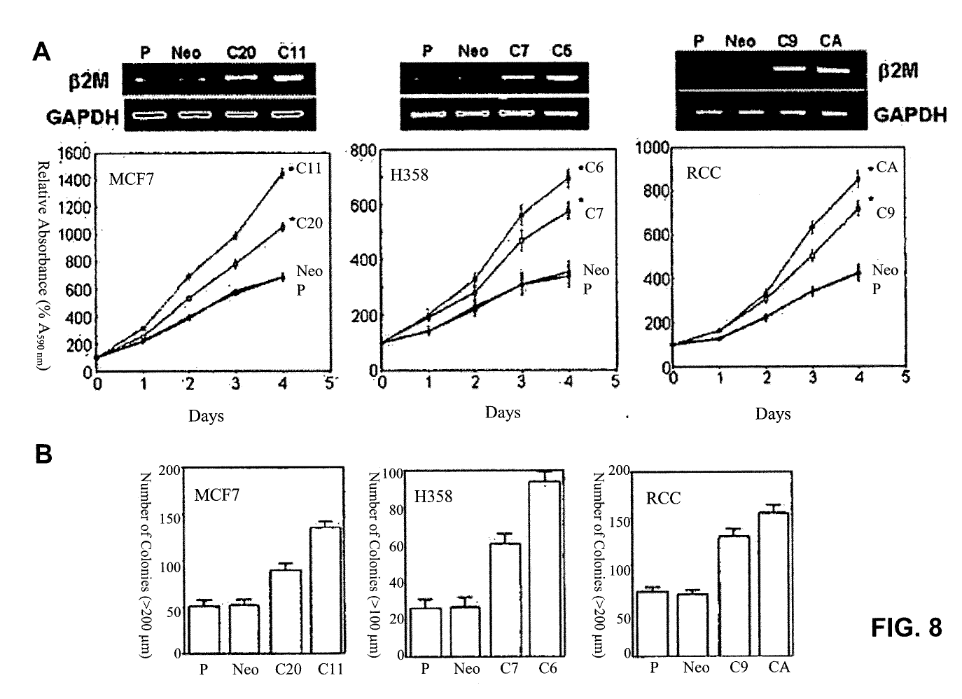

Because breast, lung and renal cancers are known to metastasize to bone, we performed similar studies to test the possible roles of β2M in stimulating the growth of these human cancer cell lines in culture and in soft agars. FIG. 8 shows that the levels of β2M expression correlate well with cell growth of human breast, lung and renal cancer cells in anchorage-dependent and -independent manner. These results support the idea that β2M may be a mitogen that play key roles in cancer cell growth and metastasized to bone.

The above observations identify a previously unrecognized role of β2M in promoting both anchorage-dependent and -independent growth of human prostate, breast, lung and renal cancer cells and growth of prostate cancer in mouse bone. What follows is a description of the osteomimicry-specific polynucleotides and nucleic acids of the invention: for example, osteomimicry regulatory region sequences (and transcriptionally active fragments thereof), in conjunction with reporter constructs utilizing such osteomimicry-specific polynucleotides and nucleic acids that may be used to screen for candidate compounds or substances capable of interfering with the expression of the heterologous coding sequence. The identified compounds or substances may be used to interfere with the ability of cancer cells to express restricted bone-like proteins, e.g., one or more of osteocalcin (OC), bone sialoprotein (BSP), SPARC/osteonectin (ON), osteopontin (OPN) and the receptor activator of NFκB ligand (RANKL).

Osteomimecry Polynucleotides and Nucleic Acids

The present invention encompasses polynucleotide sequences comprising the 5′ regulatory region, and transcriptionally active fragments thereof, of an osteomimicry gene, including osteocalcin (OC), bone sialoprotein (BSP), SPARC/osteonectin (ON), osteopontin (OPN), and the receptor activator of NFκB ligand (RANKL). The nucleotide sequences of the promoter regions of each of osteocalcin (OC) (SEQ ID NO. 1), bone sialoprotein (BSP) (SEQ ID NO. 2), SPARC/osteonectin (ON) (SEQ ID NO. 3), osteopontin (OPN) (SEQ ID NO. 4), the receptor activator of NFκB ligand (RANKL) (SEQ ID NO. 5), and the androgen receptor (AR) (SEQ ID NO. 6) are shown in the sequence listing. The promoter sequences of VEGF, NP-1 and Runx2 are available in the public domain and one of ordinary skill in the art may obtain the promoter sequences of VEGF, NP-1 and Runx2 and use such promoter sequences in the methods disclosed in the present invention without undue experimentation.

One embodiment in accordance with the invention, purified nucleic acids having at least 8 nucleotides (i.e., a hybridizing sequence) of a regulatory region, and transcriptionally active fragments thereof gene sequence are provided. In other embodiments, the nucleic acids consist of at least 20 (contiguous) nucleotides, 25 nucleotides, 50 nucleotides, 100 nucleotides, 200 nucleotides, 500, 1000, 2000, 3000, 4000 or 5000 nucleotides of an osteomimicry regulatory region sequence, or transcriptionally active fragment thereof sequence. Methods well known to those skilled in the art may be used to construct these sequences, either present in isolated form or harbored inside expression vectors. In another embodiment, the nucleic acids are smaller than 20, 25, 35, 200 or 500 nucleotides in length. Nucleic acids can be single or double stranded. The invention also encompasses nucleic acids that can hybridize with the foregoing sequences. In specific aspects, nucleic acids are provided which comprise a sequence complementary to at least 10, 20, 25, 50, 100, 200, 500 nucleotides or the entire osteomimicry regulatory region and transcriptionally active fragments gene.

The nucleotide sequences in accordance with the invention may include nucleotide sequences having at least 65%, 70%, 75%, 80%, 85%, 90%, 95%, 98%, 99% or more nucleotide sequence identity to the nucleotide sequence depicted in SEQ ID NOs. 1, 2, 3, 4, 5, and 6, and/or transcriptionally active fragments thereof, which are capable of driving expression specifically within tumor and tissue cells with calcification potential.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of positions×100). In one embodiment, the two sequences are the same length.

The determination of percent identity between two sequences may be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches may be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast may be used to perform an iterated search which detects distant relationships between molecules (Id.). When utilizing BLAST, Gapped BLAST and PSI-Blast programs, the default parameters of the respective programs (e.g., XBLAST and NBLAST) may be used (see http://www.ncbi.nlm.nih.gov). Another preferred non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12 and a gap penalty of 4 can be used. In an alternate embodiment, alignments may be obtained using the NA_MULTIPLE_ALIGNMENT 1.0 program, using a GapWeight of 5 and a GapLengthWeight of 1.

The percent identity between two sequences can be determined using techniques similar to those described above, with or without allowing gaps. In calculating percent identity, typically only exact matches are counted.

Embodiments in Accordance with the Invention May Also Include:

(a) DNA vectors that contain any of the foregoing osteomimicry regulatory sequences and/or their complements (i.e., antisense);

(b) DNA expression vectors that contain any of the foregoing osteomimicry regulatory element sequences operatively associated with a heterologous gene, such as a reporter gene; and

(c) genetically engineered host cells that contain any of the foregoing osteomimicry regulatory element sequences operatively associated with a heterologous gene such that the osteomimicry regulatory element directing the expression of the heterologous gene in the host cell.

Embodiments in accordance with the invention may also include various transcriptionally active fragments of this regulatory region. A “transcriptionally active” or “transcriptionally functional” fragment of the osteomimicry regulatory region according to the present invention refers to a polynucleotide comprising a fragment of said polynucleotide which is functional as a regulatory region for expressing a recombinant polypeptide or a recombinant polynucleotide in a recombinant cell host. For the purpose of the invention, a nucleic acid or polynucleotide is “transcriptionally active” as a regulatory region for expressing a recombinant polypeptide or a recombinant polynucleotide if said regulatory polynucleotide contains nucleotide sequences containing transcriptional information. Such sequences are operatively associated with nucleotide sequences encoding the desired polypeptide or polynucleotide.

In particular, the transcriptionally active fragments of the osteomimicry regulatory region of the present invention encompass those fragments having sufficient length to activate transcription of a heterologous gene, e.g., a reporter gene, when operatively linked to the osteomimicry regulatory sequence and transfected into tumor and tissue cells with calcification potential. Typically, the regulatory region is placed immediately 5′ to, and is operatively associated with the coding sequence. As used herein, the term “operatively associated” refers to the placement of the regulatory sequence immediately 5′ (upstream) of the reporter gene, such that trans-acting factors required for initiation of transcription, such as transcription factors, polymerase subunits and accessory proteins, may assemble at this region to allow RNA polymerase dependent transcription initiation of the reporter gene.

In one embodiment, the polynucleotide sequence may further comprise other nucleotide sequences from either the osteomimicry regulatory region and transcriptionally active fragments thereof or a heterologous gene. In another embodiment, multiple copies of a promoter sequence or a fragment thereof may be linked to each other. For example, the promoter sequence or a fragment thereof may be linked to another copy of the promoter sequence or another fragment thereof in a head to tail, head to head, or tail to tail orientation. In another embodiment, an osteotropic-specific enhancer may be operatively linked to the osteomimicry regulatory sequence, or fragment thereof, and used to enhance transcription from the construct containing the osteomimicry regulatory sequence.

Also encompassed within the scope of the invention are modifications of the osteomimicry nucleotide sequences depicted in SEQ ID Nos. 1-6, respectively, without substantially affecting its transcriptional activities. Such modifications include additions, deletions and substitutions. In addition, any nucleotide sequence that selectively hybridizes to the complement of the sequence depicted in SEQ ID Nos. 1-6, respectively, under stringent conditions, and is capable of activating the expression of a coding sequence specifically within tumor and tissue cells with calcification potential is encompassed by the invention. Exemplary moderately stringent and high stringency hybridization conditions include those described in Ausubel F. M. et al., eds., 1989, Current Protocols in Molecular Biology, Vol. 1, Green Publishing Associates, Inc., and John Wiley & sons, Inc., New York, at p. 2.10.3. Other conditions of high stringency well known in the art may also be used.

The osteomimicry regulatory region, or transcriptionally functional fragments thereof, is preferably derived from a mammalian organism. Screening procedures using nucleic acid hybridization allow isolation of gene sequences from various organisms. The isolated polynucleotide sequence disclosed herein, or fragments thereof may be labeled and used to screen a cDNA library constructed from mRNA obtained from appropriate cells or tissues (e.g., calcified tissue) derived from the organism of interest. The hybridization conditions used should be of a lower stringency when the cDNA library is derived from an organism different from the type of organism from which the labeled sequence is derived. Further, mammalian osteomimicry regulatory region homologues may be isolated from, for example, bovine or other non-human nucleic acid, by performing polymerase chain reaction (PCR) amplification using two primer pools designed on the basis of the nucleotide sequence of the osteomimicry regulatory region disclosed herein. The template for the reaction may be cDNA obtained by reverse transcription of the mRNA prepared from, for example, bovine or other non-human cell lines, or tissue known to express the osteomimicry gene. For guidance regarding such conditions, see, e.g., Innis et al. (Eds.) 1995, PCR Strategies, Academic Press Inc., San Diego; and Erlich (ed) 1992, PCR Technology, Oxford University Press, New York, each of which is incorporated herein by reference in its entirety.

Promoter sequences within the 5′ non-coding regions of the osteomimicry gene may be further defined by constructing nested 5′ and/or 3′ deletions using conventional techniques such as exonuclease III or appropriate restriction endonuclease digestion. The resulting deletion fragments may be inserted into the promoter reporter vector to determine whether the deletion reduces or obliterates promoter activity such as described, for example, by Coles et al. (Hum. Mol. Genet., 7:791-800, 1998). In this way, the boundaries of the promoters may be defined. If desired, potential individual regulatory sites within the promoter may be identified using site directed mutagenesis or linker scanning to obliterate potential transcription factor binding sites within the promoter individually or in combination. The effects of these mutations on transcription levels may be determined by inserting the mutations into cloning sites in promoter reporter vectors. These types of assays are well known to those skilled in the art (WO 97/17359, U.S. Pat. No. 5,374,544, EP 582 796, U.S. Pat. No. 5,698,389, U.S. Pat. No. 5,643,746, U.S. Pat. No. 5,502,176, and U.S. Pat. No. 5,266,488).

The osteomimicry regulatory regions and transcriptionally functional fragments thereof, and the fragments and probes described herein which serve to identify osteomimicry regulatory regions and fragments thereof, may be produced by recombinant DNA technology using techniques well known in the art. Methods well known to those skilled in the art may be used to construct these sequences, either in isolated form or contained in expression vectors. These methods include, for example, in vitro recombinant DNA techniques, synthetic techniques and in vivo genetic recombination. See, e.g., the techniques described in Sambrook et al., 1989, and Ausabel et al, 1989; also see the techniques described in “Oligonucleotide Synthesis”, 1984, Gait M. J. ed., IRL Press, Oxford, which is incorporated herein by reference in its entirety.

Alterations in the regulatory sequences may be generated using a variety of chemical and enzymatic methods well known to those skilled in the art. For example, regions of the sequences defined by restriction sites may be deleted. Oligonucleotide-directed mutagenesis may be employed to alter the sequence in a defined way and/or to introduce restriction sites in specific regions within the sequence. Additionally, deletion mutants may be generated using DNA nucleases such as Bal31, ExoII, or S1 nuclease. Progressively larger deletions in the regulatory sequences may be generated by incubating the DNA with nucleases for increased periods of time (see, e.g., Ausubel et al., 1989).

The altered sequences may be evaluated for their ability to direct expression of heterologous coding sequences in appropriate host cells. It is within the scope of the present invention that any altered regulatory sequences that retain their ability to direct expression of a coding sequence may be incorporated into recombinant expression vectors for further use.

### Analysis of Osteomimecry Regulatory Region Activity

The osteomimicry regulatory region sequence, or transcriptionally active fragment thereof such as, not by way of limitation, nucleotide sequences encoding the osteocalcin (OC), bone sialoprotein (BSP), SPARC/osteonectin (ON), osteopontin (OPN) and the receptor activator of NFκB ligand (RANKL) regulatory region exhibit tissue- and cell type-specificity; i.e., activates specific gene expression in osteotropic cells. Thus, the osteomimicry regulatory region sequence, and transcriptionally active fragments thereof, of the present invention may be used to induce expression of a heterologous coding sequence specifically in osteotropic cells. The activity and the specificity of the osteomimicry regulatory region sequence, and transcriptionally active fragments thereof may be determined by the expression level of polynucleotides driven by these elements in different cell types and tissues. Alternatively, cell lines engineered to harbor expression vectors containing polynucleotides driven by osteomimicry regulatory region sequence, and transcriptionally active fragments thereof may also be used. As discussed below, the polynucleotides may be either polynucleotides that specifically hybridizes with a predefined oligonucleotide probe, or polynucleotides encoding proteins. The osteomimicry regulatory region sequence, and transcriptionally active fragments thereof may be used to screen candidate compounds or substances, which have the ability to interfere with the expression of the heterologous coding sequence. The candidate compounds or substances may interfere with the expression of highly restricted bone-like proteins such as one or more of osteocalcin (OC), bone sialoprotein (BSP), SPARC/osteonectin (ON), osteopontin (OPN) and the receptor activator of NFκB ligand (RANKL) in cancer cells.

### Osteomimecry Regulatory Region Driven Reporter Constructs

The regulatory polynucleotides according to the invention may be a part of a recombinant expression vector used to express a coding sequence, or reporter gene, in a host cell or organism. The osteomimicry regulatory region sequence, and transcriptionally active fragments thereof of the present invention, and transcriptionally active fragments thereof, may be used to direct the expression of a heterologous coding sequence. In particular, the present invention encompasses mammalian osteomimicry regulatory region sequence, and transcriptionally active fragments thereof. Embodiments in accordance with the present invention include transcriptionally active fragments of the osteomimicry regulatory region sequence and transcriptionally active fragments thereof and elements with sufficient length encompassing those fragments to activate transcription of a reporter gene, to which the above fragments are operatively linked.

A variety of reporter gene sequences well known to those of skill in the art may be utilized including, but not limited to, genes encoding fluorescent proteins such as green fluorescent protein (GFP), enzymes (e.g. CAT, beta-galactosidase, luciferase) or antigenic markers. For convenience, enzymatic reporters and light-emitting reporters analyzed by colorometric or fluorometric assays are preferred reporters used in the screening assays in accordance with the invention.

In one embodiment, a bioluminescent, chemiluminescent, or fluorescent protein may be used as a light-emitting reporter. Types of light-emitting reporters, which may not require substrates or cofactors include, but are not limited to, the wild-type green fluorescent protein (GFP) of Victoria aequoria (Chalfie et al., 1994, Science 263:802-805), and modified GFPs (Heim et al., 1995, Nature 373:663-4; PCT publication WO 96/23810). Transcription and translation of this type of reporter gene may lead to accumulation of the fluorescent protein in the test cells. The fluorescence may be measured by a fluorimeter, or a flow cytometer, by methods well known in the art (see, e.g., Lackowicz, 1983, Principles of Fluorescence Spectroscopy, Plenum Press, New York).

Another type of reporter gene that may be used is enzymes that require cofactor(s) to emit light including, but not limited to, Renilla luciferase. Other sources of luciferase well known in the art include, but not limited to, bacterial luciferase (luxAB gene product) of Vibrio harveyi (Karp, 1989, Biochim. Biophys. Acta 1007:84-90; Stewart et al. 1992, J. Gen. Microbiol, 138:1289-1300) and the luciferase from firefly, Photinus pyralis (De Wet et al. 1987, Mol. Cell. Biol. 7:725-737), which may be assayed by light production (Miyamoto et al., 1987, J. Bacteriol. 169:247-253; Loessner et al 1996, Environ. Microbiol. 62:1133-1140; and Schultz & Yarus, 1990, J. Bacteriol. 172:595-602).

Reporter genes may be analyzed using colorimetric analysis such as, but are not limited to, β-galactosidase (Nolan et al. 1988, Proc. Natl. Acad. Sci. USA 85:260307), β-glucuronidase (Roberts et al. 1989, Curr. Genet. 15:177-180), luciferase (Miyamoto et al., 1987, J. Bacteriol. 169:247-253), or β-lactamase. In one embodiment, the reporter gene sequence comprises a nucleotide sequence encoding a LacZ gene product, P-galactosidase. This enzyme is stable and uses different histochemical, chromogenic or fluorogenic substrates such as, but not limited to, 5-bromo-4-chloro-3-indoyl-β-D-galactoside (X-gal), lactose 2,3,5-triphenyl-2H-tetrazolium (lactose-tetrazolium) and fluorescein galactopyranoside (see Nolan et al., 1988).

In another embodiment, the product of the E. coli β-glucuronidase gene (GUS) may be used as a reporter gene (Roberts et al. 1989, Curr. Genet. 15:177-180). GUS activity may be detected by various histochemical and fluorogenic substrates such as X-glucuronide (Xgluc) and 4-methylumbelliferyl glucuronide.

Other reporter gene sequences such as selectable reporter gene sequences may be employed. For example, the coding sequence for chloramphenicol acetyl transferase (CAT) may be constructed in a way that is driven by osteomimicry regulatory region sequence and transcriptionally active fragments thereof in a osteomimicry-dependent manner to prevent inhibition of cell growth by chloramphenicol. The use of CAT and the advantages of a selectable reporter gene are well known to those skilled in the art (Eikmanns et al. 1991, Gene 102:93-98). Other selectable reporter gene sequences may also be used that include, but are not limited to, gene sequences encoding polypeptides which confer zeocin (Hegedus et al. 1998, Gene 207:241-249) or kanamycin resistance (Friedrich & Soriano, 1991, Genes. Dev. 5:1513-1523).

Other coding sequences such as toxic gene products, potentially toxic gene products, and antiproliferation or cytostatic gene products, also may be used. In another embodiment, the reporter polynucleotides may be either polynucleotides that specifically hybridize with a predefined oligonucleotide probes or polynucleotides encoding proteins including BSP polypeptides or a fragment or a variant thereof. This type of assay is well known to those skilled in the art (U.S. Pat. No. 5,502,176 and U.S. Pat. No. 5,266,488).

Osteomimecry regulatory region sequence and transcriptionally active fragments thereof driven reporter constructs may be constructed according to standard recombinant DNA techniques (see, e.g., Methods in Enzymology, 1987, volume 154, Academic Press; Sambrook et al. 1989, Molecular Cloning—A Laboratory Manual, 2nd Edition, Cold Spring Harbor Press, New York; and Ausubel et al. Current Protocols in Molecular Biology, Greene Publishing Associates and Wiley Interscience, New York, each of which is incorporated herein by reference in its entirety).

Methods for assaying promoter activity are well-known to those skilled in the art (see, e.g., Sambrook et al., Molecular Cloning A Laboratory Manual, Cold Spring Harbor Laboratory, Cold Spring Harbor, N.Y., 1989). An example of a typical method used involves a recombinant vector carrying a reporter gene and genomic sequences from the osteomimicry regulatory region sequence depicted in SEQ ID NOs. 1-6, respectively. Briefly, the expression of the reporter gene (for example, green fluorescent protein, luciferase, β-galactosidase or chloramphenicol acetyl transferase) may be detected when placed under the control of a transcriptionally active polynucleotide fragment. Genomic sequences located upstream of the first exon of the gene may be cloned into any suitable promoter reporter vector. For example, a number of commercially available vectors may be engineered to insert the osteomimicry regulatory region sequence and transcriptionally active fragments thereof of the invention to drive gene expression in mammalian host cells. Non-limiting examples of such vectors are pSEAPBasic, pSEAP-Enhancer, pgal-Basic, pβgal-Enhancer, or pEGFP-1 Promoter Reporter vectors (Clontech, Palo Alto, Calif.) or pGL2-basic or pGL3-basic promoterless luciferase reporter gene vector (Promega, Madison, Wis.). Each of these promoter reporter vectors include multiple cloning sites positioned upstream of a reporter gene encoding a readily assayable protein such as secreted alkaline phosphatase, green fluorescent protein, luciferase or β-galactosidase. The osteomimicry regulatory region sequence, and transcriptionally active fragments thereof may be inserted into the cloning sites upstream of the reporter gene in both orientations and introduced into an appropriate host cell. The levels of reporter protein expression may be assayed and compared to that obtained with an empty vector lacking an insert in the cloning site. The presence of an elevated expression levels in the vector containing the insert with respect the control vector indicates the presence of a promoter in the insert.

Expression vectors having a osteomimicry regulatory region sequence, and transcriptionally active fragments thereof may further contain a gene encoding a selectable marker. A number of selection systems may be used including but not limited to, the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026) and adenine phosphoribosyltransferase (Lowy et al., 1980, Cell 22:817) genes, which can be employed in tk⁻, hgprt⁻ or aprt⁻ cells, respectively. Also, antimetabolite resistance may be used as the basis of selection for dhfr, which confers resistance to methotrexate (Wigler et al., 1980, Proc. Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes. Additional selectable genes include trpB, which allows cells to utilize indole in place of tryptophan; hisD, which allows cells to utilize histinol in place of histidine (Hartman & Mulligan, 1988, Proc. Natl. Acad. Sci. USA 85:8047); ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.) and glutamine synthase (Bebbington et al., 1992, Biotech 10:169).

### Characterization of Transcriptionally Active Osteomimecry Regulatory Region Sequences and Transcriptionally Active Fragments Thereof

A fusion construct comprising an osteomimicry regulatory region sequence, and transcriptionally active fragments thereof, or a fragment thereof, may be assayed for transcriptional activity. As a first step in promoter analysis, the transcriptional start point (+1 site) of the osteotropic-specific gene under study may be determined using primer extension assay and/or RNAase protection assay according to the standard methods (Sambrook et al., 1989, Molecular Cloning: A Laboratory Manual, Cold Spring Harbor, Cold Spring Harbor Press). The DNA sequence upstream from the +1 site is generally considered as the promoter region responsible for gene regulation. However, downstream sequences, including sequences within introns, may also be involved in gene regulation. To analyze the promoter activity, a −3 kb to +3 kb region (where +1 is the transcriptional start point) may be cloned upstream of the reporter gene coding region. Two or more additional reporter gene constructs may be made which contain 5′ and/or 3′ truncated versions of the regulatory region to aid in identification of the region responsible for osteotropic-specific expression. The choice of this type of reporter gene may be made based on the nature of application.

In a preferred embodiment, a GFP reporter gene construct may be used. The application of green fluorescent protein (GFP) as a reporter may be useful in the study of osteotropic-specific gene promoters. An advantage of using GFP as a reporter lies in the fact that GFP may be detected in freshly isolated tumor and tissue cells with calcification potential without the need for substrates.

In another embodiment of the invention, a Lac Z reporter construct may be used. The Lac Z gene product, β-galactosidase, may be stable and use different histochemical, chromogenic or fluorogenic substrates such as, but not limited to, 5-bromo-4-chloro-3-indoyl-β-D-galactoside (X-gal), lactose 2,3,5-triphenyl-2H-tetrazolium (lactose-tetrazolium), and fluorescein galactopyranoside (see Nolan et al., 1988).

For promoter analysis in transgenic mice, GFP may be optimized for expression in mammalian cells. A promoterless cloning vector pEGFP1 (Clontech, Palo Alto, Calif.) encodes a red shifted variant of the wild-type GFP, which may be optimized for brighter fluorescence and higher expression in mammalian cells (Cormack et al., 1996, Gene 173:33; Haas et al., 1996, Curr. Biol. 6:315). Moreover, since the maximal excitation peak of this enhanced GFP (EGFP) is at 488 nm, commonly used filter sets such as fluorescein isothiocyanate (FITC) optics that illuminate at 450-500 nm may be used to visualize GFP fluorescence. Thus, pEGFP1 may be useful as a reporter vector for promoter analysis in transgenic mice (Okabe et al, 1997, FEBS Lett. 407:313). In an alternate embodiment, transgenic mice containing transgenes, e.g., a luciferase reporter gene, driven by an osteomimicry regulatory region sequence and transcriptionally active fragments thereof may be used.

Putative osteomimicry regulatory region sequences and transcriptionally active fragments thereof may be prepared (usually from a parent phage clone containing 8-10 kb genomic DNA including the promoter region) for cloning using methods known in the art. In one embodiment, promoter fragments may be cloned into the multiple cloning site of a luciferase reporter vector. In one embodiment, restriction endonucleases may be used to excise the osteomimicry regulatory region sequence, and transcriptionally active fragments thereof to be inserted into the reporter vector. The feasibility of this method, however, depends on the availability of proper restriction endonuclease sites in the regulatory fragment. In a preferred embodiment, the required promoter fragment is amplified by polymerase chain reaction (PCR; Saiki et al., 1988, Science 239:487) using oligonucleotide primers bearing the appropriate sites for restriction endonuclease cleavage. The sequence necessary for restriction cleavage includes at the 5′ end of the forward and reverse primers which flank the regulatory fragment to be amplified. After PCR amplification, the appropriate ends may be generated by restriction digestion of the PCR product. The osteomimicry regulatory region sequence, and transcriptionally active fragments thereof, generated by either method, may be ligated into the multiple cloning site of the reporter vector following standard cloning procedures (Sambrook et al., 1989). It is recommended that the DNA sequence of the PCR generated promoter fragments in the constructs be verified prior to generation of transgenic animals. The resulting reporter gene construct may contain the putative osteomimicry regulatory region sequence, and transcriptionally active fragments thereof located upstream of the reporter gene open reading frame, e.g., GFP or luciferase cDNA. The osteomimicry regulatory region sequence and transcriptionally active fragments thereof with the reporter gene may then be used to screen candidate compounds or substances that can interfere with the expression of the heterologous coding sequence. The candidate compounds may interfere with the expression of the highly restricted bone-like proteins including one or more of osteocalcin (OC), bone sialoprotein (BSP), SPARC/osteonectin (ON), osteopontin (OPN) and the receptor activator of NFκB ligand (RANKL).

### Osteomimecry Regulatory Region Sequence Analysis Using Transgenic Mice

The mammalian osteomimicry regulatory region sequences and transcriptionally active fragments thereof may be used to direct expression of a reporter coding sequence, a homologous gene, or a heterologous gene in transgenic animals specifically within tumor and tissue cells with calcification potential. Animals of any species including, but not limited to, mice, rats, rabbits, guinea pigs, pigs, micro-pigs, goats, sheep, and non-human primates, e.g., baboons, monkeys and chimpanzees may be used to generate transgenic animals. The term “transgenic,” as used herein, refers to non-human animals expressing osteomimicry regulatory region and transcriptionally active fragments thereof from a different species (e.g., mice expressing human osteomimicry regulatory region and transcriptionally active fragments thereof sequences), as well as animals that have been genetically engineered to over-express endogenous (i.e., same species) osteomimicry regulatory region and transcriptionally active fragments thereof sequences or animals that are genetically engineered to knock-out specific sequences.

In one embodiment according to the present invention, transgenic animals carry a transgene such as a reporter gene, therapeutic and/or toxic coding sequence under the control of the osteomimicry regulatory region and transcriptionally active fragments thereof, expressed in all or in some (mosaic animals) of their cells. The transgene may be integrated as a single transgene or in concatamers, e.g., head-to-head tandems or head-to-tail tandems. The transgene may also be selectively introduced into and activated in a particular cell type by following, for example, the teaching of Lasko et al. (1992, Proc. Natl. Acad. Sci. USA 89:6232-6236). The transgene may be integrated into the chromosomal site of the endogenous corresponding gene by gene targeting technique well known in the art. Briefly, vectors containing some nucleotide sequences homologous to the endogenous gene may be designed for the purpose of integrating into specific chromosomal locations via homologous recombination. As a result, the chromosomal insertion may disrupt the function of the endogenous genes at the sites of insertion.

Any technique known in the art may be used to introduce a transgene under the control of the osteomimicry regulatory region and transcriptionally active fragments thereof into animals to produce the founder lines of transgenic animals. Such techniques include, but are not limited to, pronuclear microinjection (Hoppe & Wagner, 1989, U.S. Pat. No. 4,873,191); nuclear transfer into enucleated oocytes of nuclei from cultured embryonic, fetal or adult cells induced to quiescence (Campbell et al., 1996, Nature 380:64-66; Wilmut et al., Nature 385:810-813); retrovirus gene transfer into germ lines (Van der Putten et al., 1985, Proc. Natl. Acad. Sci., USA 82:6148-6152); gene targeting in embryonic stem cells (Thompson et al., 1989, Cell 65:313-321); electroporation of embryos (Lo, 1983, Mol. Cell. Biol. 31:1803-1814); and sperm-mediated gene transfer (Lavitrano et al., 1989, Cell 57:717-723; see, Gordon, 1989, Transgenic Animals, Intl. Rev. Cytol. 115:171-229).

For example, a linear DNA fragment (a transgene) containing the regulatory region, the reporter gene, and the polyadenylation signals, may be excised from the reporter gene construct. The transgene may be gel purified by methods known in the art, e.g., electroelution. Following electroelution, any traces of impurities may be further removed from the transgene fragments by passing through Elutip D column (Schleicher & Schuell, Dassel, Germany).

In a preferred embodiment, the purified transgene fragment may be microinjected into the male pronuclei of fertilized eggs obtained from B6 CBA females by standard methods (Hogan, 1986, Manipulating the Mouse Embryo, A Laboratory Manual. Cold Spring Harbor Laboratory Press, Cold Spring Harbor, N.Y.). Mice may be analyzed transiently at several embryonic stages or by establishing founder lines that allow more detailed analysis of transgene expression throughout development and in adult animals. The presence of transgene may be verified by PCR using genomic DNA isolated from placentas (transients) or tail clips (founders) according to the method of Vemet et al., Methods Enzymol. 1993; 225:434-451. Preferably, the PCR reaction may be carried out in a volume of 100 μl containing 1 μg of genomic DNA, in 1× reaction buffer supplemented with 0.2 mM dNTPs, 2 mM MgCl₂, 600 μM each of primer, and 2.5 units of Taq polymerase (Promega, Madison, Wis.). Each of the 30 PCR cycles may be performed, for example, using the steps of denaturation at 94° C. for 1 min, annealing at 54° C. for 1 min, and extension at 72° C. for 1 min. The founder mice may be mated with C57B1 partners to generate transgenic F₁ lines of mice.

Screening Assays for Compounds or Substances that Modulate Osteomimicry

Compounds or substances that exhibit ability to interfere with the abnormal function and/or growth of tumor and tissue cells with calcification potential may be useful to treat defects in osteotropic-related disorders. These disorders include, but not limited to, localized or disseminated osteosarcoma, lung, renal, colon, melanoma, thyroid, brain, multiple myeloma, breast and prostate cancers, and benign conditions, such as benign prostatic hyperplasia (BPH) or arterial sclerotic conditions where calcification occurs. Such compounds may also be used to interfere with the onset or the progression of osteotropic-related disorders. Compounds or substances that stimulate or inhibit promoter activity may be used to ameliorate symptoms of osteotropic-related disorders.

Genetically engineered cells, cell lines, and/or transgenic animals containing a osteomimicry regulatory region and transcriptionally active fragments thereof, operatively linked to a reporter gene, may be used to screen agents that have potentials to modulate osteomimicry regulatory region and transcriptionally active fragments thereof activity. Such transgenic mice may provide an in vivo assay (or may be used as a source of primary cells or cell lines for use in vitro) to identify new methods for treating osteotropic-related disorders by targeting therapeutic agents to inhibit the progression of such disorders.

Embodiments in accordance with the present invention include screening assays for identifying compounds or substances that have potentials to modulate activity of the osteomimicry regulatory region and transcriptionally active fragments thereof. The embodiments include in vitro cell-based assays and in vivo assays using transgenic animals. As described below, test compounds include, but are not limited to, oligonucleotides, peptides, proteins, small organic or inorganic compounds, antibodies, etc.

Examples of compounds include, but are not limited to, peptides such as soluble peptides. The peptides further include, but not limited to, Ig-tailed fusion peptides, and members of random peptide libraries (see, e.g., Lam, et al, 1991, Nature 354:82-84; Houghten, et al., 1991, Nature 354:84-86), and combinatorial chemistry-derived molecular library made of D- and/or L-configuration amino acids, phosphopeptides (including, but not limited to members of random or partially degenerate, directed phosphopeptide libraries; see, e.g., Songyang, et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

The compounds may further include drugs or members of classes or families of drugs known to ameliorate the symptoms of an osteotropic-related disorder. These compounds include, but are not limited to, families of antidepressants such as lithium salts, carbamazepine, valproic acid, lysergic acid diethylamide (LSD), pchlorophenylalanine, p-propyldopacetamide dithiocarbamate derivatives, e.g., FLA 63; antianxiety drugs, e.g., diazepam; monoamine oxidase (MAO) inhibitors, e.g., iproniazid, clorgyline, phenelzine and isocarboxazid; biogenic amine uptake blockers, e.g., tricyclic antidepressants such as desipramine, imipramine and amitriptyline; serotonin reuptake inhibitors, e.g., fluoxetine; antipsychotic drugs such as phenothiazine derivatives (e.g., chlorpromazine (thorazine) and trifluopromazine)), butyrophenones (e.g., haloperidol (Haldol)), thioxanthene derivatives (e.g., chlorprothixene), and dibenzodiazepines (e.g., clozapine); benzodiazepines; dopaminergic agonists and antagonists e.g., L-DOPA, cocaine, amphetamine, .alpha.-methyl-tyrosine, reserpine, tetrabenazine, benzotropine, pargyline; noradrenergic agonists and antagonists e.g., clonidine, phenoxybenzamine, phentolamine, tropolone; nitrovasodilators (e.g., nitroglycerine, nitroprusside as well as NO synthase enzymes); and antagosists of growth factors (e.g., VEGF, FGF, angiopoetins and endostatin), androgen receptor antagonists, GPCR antagonists, PKA/CREB signal activation interrupters, β2M/PKA/CREB signaling interupters, CREB transcription factor, and complex formation signal activation interrupters, or any combination thereof.

In one preferred embodiment, genetically engineered cells, cell lines, or primary cultures of germ and/or somatic cells containing a mammalian osteomimicry regulatory region and transcriptionally active fragments thereof operatively linked to a heterologous gene may be used to develop screening assays to identify compounds that can inhibit sequence-specific DNA-protein interactions. Such methods may include (1) contacting a cell with a compound or substance to expresses a gene under the control of a osteomimicry regulatory region and transcriptionally active fragments thereof, (2) measuring the level of the gene expression or gene product activity, and (3) comparing this level to the level of gene expression or gene product activity produced by the cell in the absence of the compound or substance. Candidate compounds may be identified when the levels of gene expression in the presence of the compound or substance differ from that in the absence of the compound. Changes in gene expression levels may be monitored by using any number of methods known to those of skill in the art, e.g., reporter gene activity, mRNA levels (e.g. Northern blot analysis) or using other methods known in the art to quantify the levels of gene products expressed in the cell.

In another embodiment, microdissection and transillumination may be used. These techniques may provide a rapid assay for monitoring the effects of putative drugs on osteotropic cells in transgenic animals containing a osteomimicry regulatory region and transcriptionally active fragments thereof—driven reporter gene. In this embodiment, a test agent may be delivered to the transgenic animal by any number of methods. Methods for introducing a test agent into the animals may include oral, intradermal, intramuscular, intraperitoneal, intravenous, subcutaneous, intranasal and via scarification (scratching through the top layers of skin, e.g., using a bifurcated needle) or any other standard routes of drug delivery. The effect of such test compounds on the osteotropic cells may be analyzed by the microdissection and transillumination of the osteoblastic cells. A candidate compound may be identified, if the levels of reporter gene expression observed or measured in the presence of the compound differ from that obtained in the absence of the compound.

In various embodiments of the invention, the compounds screened for their ability to modulate the osteotropic-related disorders (via interfering with osteomimicry regulatory region and transcriptionally active fragments thereof) include peptides, small molecules, both naturally occurring and/or synthetic (e.g., libraries of small molecules or peptides), cell-bound or soluble molecules, organic, non-protein molecules and recombinant molecules. Further, the proteins and compounds may include endogenous cellular components that interact with osteomimicry regulatory region and transcriptionally active fragments thereof sequences in vivo. Cell lysates or tissue homogenates may be screened for proteins or other compounds which bind to the osteomimicry regulatory region and transcriptionally active fragments thereof. Such endogenous components may provide new targets for pharmaceutical and therapeutic interventions.

In one embodiment, libraries, e.g., peptide libraries, chemically synthesized libraries, recombinant (e.g., phage display libraries), and in vitro translation-based libraries known in the art, may be used to screen candidate compounds. In one embodiment of the present invention, peptide libraries may be used to screen for agonists or antagonists of osteomimicry regulatory region and transcriptionally active fragments thereof—linked reporter expression. Diversity libraries such as random or combinatorial peptide or non-peptide libraries may be screened for molecules that specifically modulate osteomimicry regulatory region and transcriptionally active fragments thereof activity. Random peptide libraries containing all possible combinations of amino acids attached to a solid phase support may be used to identify peptides that can activate or inhibit osteomimicry regulatory region and transcriptionally active fragments thereof activities (Lam, K. S. et al., 1991, Nature 354:82-84). Screening the peptide libraries may identify therapeutic agents that either stimulate or inhibit the expression of osteomimicry regulatory region and transcriptionally active fragments thereof.

Examples of chemically synthesized libraries are described in Fodor et al., 1991, Science 251:767-773; Houghten et al., 1991, Nature 354:84-86; Lam et al., 1991, Nature 354:82-84; Medynski, 1994, BioTechnology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al, 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott and Smith, 1990, Science 249:386-390; Devlin et al., 1990, Science, 249:404-406; Christian, et al., 1992, J. Mol. Biol. 227:711-718; Lenstra, 1992, J. Immunol. Meth. 152:149-157; Kay et al., 1993, Gene 128:59-65; and PCT Publication No. WO 94/18318 dated Aug. 18, 1994.

By way of example of non-peptide libraries, a benzodiazepine library (see e.g., Bunin et al., 1994, Proc. Natl. Acad. Sci. USA 91:4708-4712) may be adapted for use. Peptoid libraries (Simon et al., 1992, Proc. Natl. Acad. Sci. USA 89:9367-9371) may also be used. Another example of a library, in which the amide functionalities in peptides are permethylated to generate a chemically transformed combinatorial library, as described by Ostresh et al. (1994, Proc. Natl. Acad. Sci. USA 91:11138-11142) may be used.

The osteomimicry regulatory region and transcriptionally active fragments thereof—reporter vector may be used to generate transgenic mice. Primary cultures of osteomimicry regulatory region and transcriptionally active fragments thereof—reporter vector germ cells may be established from the transgenic mice. About 10,000 cells per well may be plated in 96-well plates in total volume of 100 μl. Candidate inhibitors of the osteomimicry regulatory region and transcriptionally active fragments thereof may be added to the cells. The effect of the inhibitors of the osteomimicry regulatory region and transcriptionally active fragments thereof may be determined by measuring the response of the reporter gene driven by the osteomimicry regulatory region and transcriptionally active fragments thereof. This assay may easily be set up in a high-throughput screening mode to screen the compound libraries in a 96-well format by quantifying the reporter gene activity. After 6 hours of incubation, 100 μl DMEM medium +2.5% fetal bovine serum (FBS) to 1.25% final serum concentration may be added to the cells and incubated for a total of 24 hours (18 hours more). At 24 hours, the plates may be washed with PBS, blot dried, and frozen at −80° C. The plates may be thawed the next day and analyzed for the presence of reporter activity.

In a preferred example of an in vivo screening assay, tumor or tissue cells with calcification potential derived from transgenic mice may be transplanted into mice with a normal or other desired phenotype (Brinster et al., 1994, Proc. Natl. Acad. Sci. USA 91:11298-302; Ogawa et al., 1997, Int. J. Dev. Biol. 41:111-12). Such mice may then be used to test the effect of compounds and other various factors on osteotropic-related disorders. In addition to the compounds and agents listed above, such mice may be used to assay factors or conditions that can be difficult to test using other methods, such as dietary effects, internal pH, temperature, etc.

Once a compound is identified, it may then be tested in an animal-based assay to determine if the compound exhibits the ability to ameliorate and/or prevent symptoms of an osteotropic-related disorder including, but not limited to, localized or disseminated osteosarcoma, lung, renal, colon, melanoma, thyroid, brain, multiple myeloma, breast and prostate cancers, and benign conditions such as benign prostatic hyperplasia (BPH), or arterial sclerotic conditions with calcification.

The assays of the present invention may be first optimized on a small scale (i.e., in test tubes), and then scaled up for high-throughput assays. The screening assays of the present invention may be performed in vitro using purified components or cell lysates. The screening assays of the present invention may also be carried out in intact cells and in animal models. Embodiments in accordance with the present invention include test compounds that can modulate the activity of the osteomimicry regulatory region and transcriptionally active fragments thereof in vitro. The candidate compounds may then be assayed in vivo in cultured cells and animal models to determine if the test compounds have the similar effects in vivo. Importantly, the in vivo assays may determine the effects of the candidate compounds on osteotropic-related disorders.

Osteomimicry Modulatory Antisense, Ribozyme and Triple Helix Approaches

In another embodiment, the types of conditions, disorders, or diseases involving tumor and tissue cells with calcification potential may be prevented, delayed, or rescued by modulating osteotropic-specific gene expression using a osteomimicry regulatory region and/or transcriptionally active fragments thereof in combination with well-known antisense, gene “knock-out,” ribozyme and/or triple helix methods. Such molecules may be designed to modulate, reduce, or inhibit mutant osteotropic gene activity. Techniques for the production and use of such molecules are well known to those of skill in the art.

Antisense RNA and DNA molecules may block the translation of mRNA to protein by hybridizing with the targeted mRNA. Antisense approaches may involve the design of oligonucleotides complementary to an mRNA sequence. The antisense oligonucleotides may bind to the complementary mRNA sequence transcripts and inhibit translation. Complete complementary, although preferred, is not absolutely required.

A sequence “complementary” to a portion of an RNA, as referred to herein, means a sequence having sufficient complementary to hybridize with the RNA, forming a stable duplex. In the case of double-stranded antisense nucleic acids, a single strand of the duplex DNA or triplex formation may be assayed. The ability to hybridize may depend on both the degree of complementary and the length of the antisense nucleic acid. Generally, the longer the hybridizing nucleic acid, the more base mismatches with an RNA may occur, although a stable duplex (or triplex) may form. One skilled in the art may ascertain a tolerable degree of mismatches using of standard procedures to determine the melting point of the hybridized complex.

In one embodiment, oligonucleotides complementary to non-coding regions of the sequence of interest could be used in an antisense approach to inhibit translation of endogenous mRNA. Antisense nucleic acids may be at least six nucleotides in length, and may be preferably oligonucleotides ranging from 6 to about 50 nucleotides in length. In specific aspects, the oligonucleotide is at least 10 nucleotides, at least 17 nucleotides, at least 25 nucleotides or at least 50 nucleotides.

It is preferable that the in vitro studies may be first performed to determine the inhibitory ability of the antisense oligonucleotide. It is preferred that these studies utilize the controls that can distinguish between antisense gene inhibition and nonspecific biological effects of oligonucleotides. The results obtained from using the antisense oligonucleotide may be compared with those using a control oligonucleotides. It is preferred that the control oligonucleotides are of approximately the same length as the test oligonucleotides.

The oligonucleotides may be DNA or RNA or chimeric mixtures or derivatives or modified versions thereof, single-stranded, or double-stranded. The oligonucleotides may be modified at the base moiety, sugar moiety, or phosphate backbone to improve stability of the molecule, efficiency of hybridization, etc. The oligonucleotides may include other appended groups such as peptides (e.g., for targeting host cell receptors in vivo), or agents facilitating transport across the cell membrane (see, e.g., Let singer, et al., 1989, Proc. Natl. Acad. Sci. U.S.A. 86:6553-6556; Lemaitre, et al., 1987, Proc. Natl. Acad. Sci. U.S.A. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6:958-976) or intercalating agents (see, e.g., Zon, 1988, Pharm. Res. 5:539-549). To this end, the oligonucleotides may be conjugated to another molecule, e.g., a peptide, hybridization triggered cross-linking agent, transport agent, hybridization-triggered cleavage agent, etc.

The antisense oligonucleotide may include at least one modified base moiety which may be selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosin-e, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

The antisense oligonucleotide may also include at least one modified sugar moiety selected from the group including but not limited to arabinose, 2-fluoroarabinose, xylulose, and hexose.

In yet another embodiment, the antisense oligonucleotide may include at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide may be an α-anomeric oligonucleotide. An α-anomeric oligonucleotide may form specific double-stranded hybrids with complementary RNA in which, contrary to the usual β-units, the strands run parallel to each other (Gautier, et al., 1987, Nucl. Acids Res. 15:6625-6641). The oligonucleotide may be a 2′-0-methylribonucleotide (Inoue, et al., 1987, Nucl. Acids Res. 15:6131-6148), or a chimeric RNA-DNA analogue (Inoue, et al., 1987, FEBS Lett. 215:327-330).

Embodiments of oligonucleotides in accordance with the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein, et al. (1988, Nucl. Acids Res. 16:3209), methylphosphonate oligonucleotides may be prepared by use of controlled pore glass polymer supports (Sarin, et al., 1988, Proc. Natl. Acad. Sci. U.S.A. 85:7448-7451), etc.

While antisense nucleotides complementary to an osteotropic-specific coding region sequence may be used, those complementary to the transcribed, untranslated region (for example, osteomimicry regulatory region and/or transcriptionally active fragments thereof) are preferred.

Antisense molecules may be delivered to the cells that express the osteotropic sequence in vivo. A number of methods may be used for systemically delivering antisense DNA or RNA into cells, e.g., antisense molecules injected directly into the tissue site, or modified antisense molecules designed to target the desired cells (e.g., antisense linked to peptides or antibodies which specifically bind receptors or antigens expressed on the target cell surface).

A preferred approach to achieve sufficient intracellular concentrations of the antisense to suppress translation of endogenous mRNAs may be the use a recombinant DNA construct, in which the antisense oligonucleotide is under the control of a strong pol III or pol II promoter. The use of such a construct to transfect target cells in the patient may result in the transcription of sufficient amounts of single stranded RNAs that, in turn, may form complementary base pairs with the endogenous sequence transcripts blocking the translation. For example, a vector may be introduced to direct the transcription of an antisense RNA in cells. Such a vector may be episomal or chromosomally integrated, as long as it can be transcribed to produce the desired antisense RNA. Such vectors may be constructed by recombinant DNA technology methods known in the art. Vectors used for replication and expression in mammalian cells may be plasmid, viral, or others known in the art. Expression of the sequences encoding the antisense RNAs may be driven by any promoter known in the art to act in mammalian, preferably human cells. Such promoters may be inducible or constitutive. Such promoters include, but are not limited to, the SV40 early promoter region (Bemoist and Chambon, 1981, Nature 290:304-310), the promoter contained in the 3′-long terminal repeat of Rous sarcoma virus (Yamamoto, et al., 1980, Cell 22:787-797), herpes thymidine kinase promoter (Wagner, et al., 1981, Proc. Natl. Acad. Sci. U.S.A. 78:1441-1445), the regulatory sequences of the metallothionein gene (Brinster, et al., 1982, Nature 296:39-42), etc. Any type of plasmid, cosmid, YAC or viral vector may be used to prepare the recombinant DNA constructs used for introduction into the tissues. Alternatively, viral vectors may be used to infect the specific tissues. In this case, administration may be accomplished systemically.

Ribozyme molecules may be designed to catalytically cleave target gene mRNA transcripts and thus inhibiting translation of the target gene mRNAs (See, e.g., PCT International Publication WO90/11364, published Oct. 4, 1990; Sarver, et al, 1990, Science 247, 1222-1225).

Ribozymes are enzymatic RNA molecules capable of catalyzing the specific cleavage of RNA. (For a review, see Rossi, 1994, Current Biology 4:469-471). The mechanism of ribozyme action may involve sequence specific hybridization of the ribozyme molecule to complementary target RNA, followed by an endonucleolytic cleavage event. The composition of ribozyme molecules may include one or more sequences complementary to the target gene mRNA, and the well known catalytic sequence responsible for mRNA cleavage. For this sequence, see, e.g., U.S. Pat. No. 5,093,246, which is incorporated herein by reference in its entirety.

While a variety of ribozymes may be used to destroy target gene mRNAs, the hammerhead ribozymes is preferred. Hammerhead ribozymes may cleave mRNAs at locations dictated by flanking regions, in which form complementary base pairs with the target mRNA. The sole requirement may be that the target mRNA has the following two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334:585-591, which is incorporated herein by reference in its entirety.

Preferably the ribozyme may be engineered so that the cleavage recognition site is located near the 5′ end of the target gene mRNA, i.e., to increase efficiency and minimize the intracellular accumulation of non-functional mRNA transcripts. The ribozymes of the present invention may include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been and Cech, 1986, Cell, 47:207-216). The Cech-type ribozymes may have an eight base pair active site that hybridizes to a target RNA sequence, where cleavage of the target RNA takes place. Embodiments of the invention encompass those Cech-type ribozymes targeting the eight base-pair active site sequences, which are present in the target gene.

As in the antisense approach, the ribozymes may contain modified oligonucleotides (e.g., for improved stability, efficiency of targeting, etc.) and may be delivered to cells in vivo. A preferred method of delivery involves using a DNA construct “encoding” the ribozyme under the control of a strong constitutive pol III or pol II promoter, so that the transfected cells may produce sufficient quantities of the ribozyme to destroy endogenous target mRNA and inhibit translation. Because ribozymes, unlike antisense molecules, are catalytic, a lower intracellular concentration may be required for efficiency.

Endogenous target gene expression may also be reduced by inactivating or “knocking out” the target gene or its promoter using targeted homologous recombination (e.g., see Smithies, et al, 1985, Nature 317:230-234; Thomas and Capecchi, 1987, Cell 51:503-512; Thompson, et al., 1989, Cell 5:313-321; each of which is incorporated by reference herein in its entirety). For example, a mutant, non-functional targeting vector (or a completely unrelated DNA sequence) flanked by DNA homologous sequences to either the coding regions or regulatory regions of the target gene may be used to transfect cells that express the target gene in vivo. This targeting vector may or may not have a positive or negative selectable marker. As a result, the targeting sequences may inactivate the endogenous target genes by inserting the target vector sequences into the target genes via homologous recombination. Such approaches may be particularly suited in the agricultural field where modifications to ES (embryonic stem) cells may be used to generate animal offspring with an inactive target gene (e.g., see Thomas and Capecchi, 1987 and Thompson, 1989, supra). In addition, this approach may be adapted for use in humans provided that the recombinant DNA constructs are directly administered or targeted to the target site in vivo using appropriate viral vectors.

Alternatively, endogenous target gene expression may be reduced by targeting DNA sequences complementary to the regulatory region of the target gene (i.e., the target gene promoter and/or enhancers) to form triple helical structures (triplex). The DNA triplexes may inhibit the transcription of target genes in specific cell types in vivo. (See generally, Helene, 1991, Anticancer Drug Des., 6(6):569-584; Helene, et al., 1992, Ann. N.Y. Acad. Sci., 660:27-36; and Maher, 1992, Bioassays 14(12):807-815).

Nucleic acid molecules used to form triplex may be single-stranded and composed of deoxynucleotides. The base composition of these oligonucleotides may be designed to promote triplex formation via Hoogsteen base pairing rules, which may require sizable stretches of either purines or pyrimidines present on one strand of a duplex. Nucleic acids may be pyrimidine-based, which may result in TAT and CGC+triplets across the three associated strands of the triplex. The pyrimidine-rich molecules may provide the bases complementary to a purine-rich region of a single strand in the duplex. In addition, nucleic acid molecules may be chosen which are purine-rich, e.g., a stretch of G residues. These molecules may form triplexes with a DNA duplex rich in GC pairs. The majority of the purine residues may be located on a single strand of the targeted duplex, thus, forming GGC triplets across the three strands in the triplexes.

Alternatively, the potential sequences useful for triple helix formation may be increased by creating so-called “switchback” nucleic acid molecules. Switchback molecules may be synthesized in an alternating 5′-3′,3′-5′ manner, such that they form base pair with the first strand of a duplex and then the other. Thus, it may not be necessary to have a sizable stretch of purines or pyrimidines present on a strand of the duplex.

To inhibit mutant gene expression using antisense, ribozyme, and/or triplex-forming molecules, it is possible that the transcription (by triplex-forming molecules) and/or the translation (by antisense or ribozyme) of normal gene expression may be simultaneously inhibited. To restore the normal gene expression, nucleic acid molecules encoding for the target gene polypeptides (which do not contain target sequences for antisense, ribozyme, or triplex-forming molecules) may be introduced into cells using gene therapy methods. If the target gene encodes an extracellular protein, it may be preferable to co-administer such protein to maintain the normal physiological function of the protein.

Anti-sense RNA and DNA, ribozyme, and triplex-forming molecules of the invention may be prepared by any method known in the art for the synthesis of DNA and RNA molecules. These methods may include chemically synthesizing oligodeoxyribonucleotides and oligoribonucleotides well known in the art such as solid-phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors driven by RNA polymerase promoters such as the T7 or SP6 polymerase promoters. In addition, antisense cDNA constructs driven by constitutive or inducible promoters may be used to generate stable cell lines, which may, in turn, produce antisense RNA.

### Gene Replacement Therapy

One or more copies (or a portion) of a normal gene may be delivered to the specific cell types in patients by using vectors including, but not limited to, adenovirus, adeno-associated virus, and retrovirus vectors, and other particles such as liposomes. Methods for introducing genes into mammalian cells are well known in the field. For example, using gene therapy methods, the nucleic acids may be directly administered in vivo into a target cell or a transgenic mice that express a osteomimetic-cancer specific regulatory region operatively linked to a heterologous coding sequence. This may be accomplished by any methods known in the art. For example, a vector expressing the normal gene may be administered so that it becomes intracellular, delivered by using a defective or attenuated retroviral or other viral vector (see U.S. Pat. No. 4,980,286), by direct injection of naked DNA, by using microparticle bombardment (e.g., a gene gun; Biolistic, DuPont), by coating with lipids or cell-surface receptors or transfecting agents, by encapsulated inside liposomes, microparticles, or microcapsules, by linking to a peptide which is known capable of entering the nucleus, or by linking to a ligand subject to receptor-mediated endocytosis (see e.g., Wu and Wu, 1987, J. Biol. Chem. 262:4429-4432). The receptor-mediated endocytosis approach may be useful to target the cell types specifically expressing the receptors. In another embodiment, a nucleic acid-ligand complex may be formed. The ligands may contain fusogenic viral peptides to disrupt endosomes, thus, allowing the nucleic acids to avoid lysosomal degradation. In yet another embodiment, the nucleic acids may be targeted in vivo for cell specific uptake and expression by targeting specific receptors (see, e.g., PCT Publications WO 92/06180 dated Apr. 16, 1992; WO 92/22635 dated Dec. 23, 1992; WO92/20316 dated Nov. 26, 1992; WO93/14188 dated Jul. 22, 1993; WO 93/20221 dated Oct. 14, 1993). Alternatively, the nucleic acids may be delivered into cells and incorporated into host DNA by homologous recombination (Koller and Smithies, 1989, Proc. Natl. Acad. Sci. USA 86:8932-8935; Zijlstra et al., 1989, Nature 342:435-438).

In one embodiment, gene delivery techniques involve direct administration, e.g., by stereotactic delivery of such gene sequences to cells, in which the gene sequences are to be expressed. Other methods used to increase the overall level of gene expression and/or gene product activity may include using homologous recombination methods to modify the expression characteristics of an endogenous gene in a cell or microorganism. By inserting heterologous DNA regulatory elements such that the inserted regulatory elements are operatively linked with the endogenous gene of interest, homologous recombination may thus be used to activate transcription of an endogenous gene that may be otherwise “transcriptionally silent”, e.g., normally inactive or expressed at low levels. This approach may also be used to enhance the normal expression levels of the endogenous gene.

Further, the overall levels of the target gene expression and/or gene product activity may be increased by delivering cells (preferably autologous cells) engineered to express the target gene to patients to ameliorate the symptoms of osteotropic-related disorders. Such cells may be either recombinant or non-recombinant. If the cells are non-autologous, they may be administered using well known techniques to prevent host immune response. For example, the cells may be introduced in an encapsulated form. While allowing exchange of components with the immediate extracellular environment, the introduced cells are shielded from being recognized by the host immune system.

Compounds or substances capable of modulating the activity of an osteomimicry regulatory region and transcriptionally active fragments thereof may be administered using the standard techniques well known to those of skill in the art.

Combination Therapies for Targeting Osteomimicry

In the aforementioned embodiments in accordance with the invention, combination therapies are also specifically contemplated herein. In particular, the compositions of the present invention may be administered in combination with one or more macrolide or non-macrolide antibiotics, anti-bacterial agents, anti-fungicides, anti-viral agents, and anti-parasitic agents, anti-inflammatory or immunomodulatory drugs or agents.

Examples of macrolide antibiotics that may be used in combination with the composition of the present invention include, but not limited to, the following synthetic, semi-synthetic or naturally occurring microlidic antibiotic compounds: methymycin, neomethymycin, YC-17, litorin, erythromycin A to F, oleandomycin, roxithromycin, dirithromycin, flurithromycin, clarithromycin, davercin, azithromycin, josamycin, kitasamycin, spiramycin, midecamycin, rokitamycin, miokamycin, lankacidin, and the derivatives of these compounds. Thus, erythromycin and compounds derived from erythromycin belong to the general class of antibiotics known as “macrolides.” Examples of preferred erythromycin and erythromycin-like compounds include: erythromycin, clarithromycin, azithromycin, and troleandomycin.

Additional antibiotics suitable for use in the methods of the present invention include, but not limited to, any molecules that prevent, inhibit, or destroy life, such as anti-bacterial agents, anti-fungicides, anti-viral agents, and anti-parasitic agents. These agents may be isolated from an organism that produces the agent or procured from a commercial source (e.g., pharmaceutical company, such as Eli Lilly, Indianapolis, Ind.; Sigma, St. Louis, Mo.), e.g., anti-TB antibiotic isoniazid (isonicotinic acid hydrazide), rifampin, ethambutol, ethionamide, streptomycin, amikacin, clofazimine, ofloxacin, levofloxacin, troveofloxacin, Pefloxacin, gatifloxacin, and moxifloxacin. Other examples of anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, oxazalidinones, and fluoroquinolones; and their various salts, acids, bases, and other derivatives.

Anti-fungal agents include, but are not limited to, caspofungin, terbinafine hydrochloride, nystatin, amphotericin B, griseofulvin, ketoconazole, miconazole nitrate, flucytosine, fluconazole, itraconazole, clotrimazole, benzoic acid, salicylic acid, and selenium sulfide.

Anti-viral agents include, but are not limited to, valgancyclovir, amantadine hydrochloride, rimantadin, acyclovir, famciclovir, foscarnet, ganciclovir sodium, idoxuridine, ribavirin, sorivudine, trifluridine, valacyclovir, vidarabin, didanosine, stavudine, zalcitabine, zidovudine, interferon alpha, and edoxudine.

Anti-parasitic agents include, but are not limited to, pirethrins/piperonyl butoxide, permethrin, iodoquinol, metronidazole, diethylcarbamazine citrate, piperazine, pyrantel pamoate, mebendazole, thiabendazole, praziquantel, albendazole, proguanil, quinidine gluconate injection, quinine sulfate, chloroquine phosphate, mefloquine hydrochloride, primaquine phosphate, atovaquone, co-trimoxazole (sulfamethoxazole/trimethoprim), and pentamidine isethionate.

In each of the aforementioned methods of the present invention, one may, for example, supplement the composition by administering a therapeutically effective amount of one or more an anti-inflammatory or immunomodulatory drugs or agents. The “immunomodulatory drugs or agents” means, for example, the agents that act on the immune system, directly or indirectly, the agents that stimulate or suppress cellular activity of cells (T-cells, B-cells, macrophages, or antigen presenting cells (APC)) in the immune system, the agents that act upon the components (e.g., hormones, receptor agonists, antagonists, neurotransmitters, and immunomodulators (e.g., immunosuppressants or immunostimulants) outside the immune system, which, in turn, may stimulate, suppress, or modulate the immune system. The “anti-inflammatory drugs” means the agents that treat inflammatory responses (a tissue reaction to injury), e.g., the agents that treat the immune, vascular, or lymphatic systems.

Anti-inflammatory or immunomodulatory drugs or agents suitable for use in this invention include, but are not limited to, interferon derivatives, e.g., betaseron, β-interferon; prostane derivatives, e.g., compounds disclosed in PCT/DE93/0013, e.g., iloprost, cicaprost; glucocorticoid, e.g., cortisol, prednisolone, methylprednisolone, dexamethasone; immunsuppressives, e.g., cyclosporine A, FK-506, methoxsalene, thalidomide, sulfasalazine, azathioprine, methotrexate; lipoxygenase inhibitors, e.g., zileutone, MK-886, WY-50295, SC-45662, SC-41661A, BI-L-357; leukotriene antagonists, e.g., compounds disclosed in DE 40091171 German patent application P 42 42 390.2; WO 9201675; SC-41930; SC-50605; SC-51146; LY 255283 (D. K. Herron et al., FASEB J. 2: Abstr. 4729, 1988); LY 223982 (D. M. Gapinski et al. J. Med. Chem. 33: 2798-2813, 1990); U-75302 and analogs, e.g., described by J. Morris et al., Tetrahedron Lett. 29: 143-146, 1988, C. E. Burgos et al., Tetrahedron Lett. 30: 5081-5084, 1989; B. M. Taylor et al., Prostaglandins 42: 211-224, 1991; compounds disclosed in U.S. Pat. No. 5,019,573; ONO-LB-457 and analogs, e.g., described by K. Kishikawa et al., Adv. Prostagl. Thombox. Leukotriene Res. 21: 407-410, 1990; M. Konno et al., Adv. Prostagl. Thrombox. Leukotriene Res. 21: 411-414, 1990; WF-11605 and analogs, e.g., disclosed in U.S. Pat. No. 4,963,583; compounds disclosed in WO 9118601, WO 9118879; WO 9118880, WO 9118883, anti-inflammatory substances, e.g., NPC 16570, NPC 17923 described by L. Noronha-Blab. et al., Gastroenterology 102 (Suppl.): A 672, 1992; NPC 15669 and analogs described by R. M. Burch et al., Proc. Nat. Acad. Sci. USA 88: 355-359, 1991; S. Pou et al., Biochem. Pharmacol. 45: 2123-2127, 1993; peptide derivatives, e.g., ACTH and analogs; soluble TNF-receptors; TNF-antibodies; soluble receptors of interleukines, other cytokines, T-cell-proteins; antibodies against receptors of interleukins, other cytokines, and T-cell-proteins.

The therapeutic agents of the instant invention may be used for the treatment of animal subjects or patients, and more preferably, mammals, including humans, as well as mammals such as non-human primates, dogs, cats, horses, cows, pigs, guinea pigs, and rodent.

Pharmaceutical Preparations and Methods of Administration

Compounds or substances that modulate osteomimicry regulatory region and transcriptionally active fragments thereof or osteomimicry gene product activity may be administered to patients at therapeutically effective doses to treat or ameliorate disorders including tumor or tissue cells with calcification potential. The therapeutically effective dose refers to that amount of the compound sufficient to result in amelioration of symptoms of such a disorder.

### Effective Dose

Toxicity and therapeutic efficacy of such compounds may be determined by standard pharmaceutical procedures using cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it may be expressed as the ratio LD₅₀/ED₅₀. Compounds that exhibit large therapeutic indices are preferred. While compounds that exhibit toxic side effects may be used, care should be taken to design a delivery system that targets such compounds to the sites of affected tissue in order to minimize potential damage to uninfected cells and, thereby, reduce side effects.

The data obtained from the cell culture assays and animal studies may be used to formulate the ranges of dosage for use in humans. The dosages of such compounds may lie preferably within a range of circulating concentrations including the ED₅₀ with little or no toxicity. The dosage may vary within this range depending upon the dosage and the route of administration. For delivering any compounds using the method of the invention, the therapeutically effective dose may be estimated initially based on the data obtained from cell culture assays. A dose may then be formulated in animal models to determine the range of circulating plasma concentration that includes the IC₅₀ (i.e., the concentration of the test compound that achieves a half-maximal inhibition of symptoms) determined in cell culture assays. Such information may be used to fine tune the useful range of doses used in humans. Levels in plasma may be measured, for example, by high performance liquid chromatography.

### Formulations and Use

Pharmaceutical compositions in accordance with the present invention may be formulated in conventional manner using one or more physiologically acceptable carriers or excipients. The compounds and their physiologically acceptable salts and solvates may be formulated for administration through inhalation, insufflation (through mouth or nose), oral, buccal, parenteral, or rectal.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated in a way to achieve controlled release of the active compound.

For buccal administration the compositions may take the form of tablets or lozenges formulated in conventional manner.

For administration by inhalation, the compounds in accordance with the present invention may be conveniently delivered in the form of an aerosol spray presentation from pressurized packs or a nebulizer, or using a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, for example, gelatin in an inhaler or insufflator may be formulated to contain a powder mix of the compound and a suitable powder base such as lactose or starch.

The compounds may be formulated for parenteral administration by injection, for example, by bolus injection or continuous infusion. Formulations for injection may be presented in unit dosage form, e.g., in ampoules or in multi-dose containers, with an added preservative. The compositions may take the forms of suspensions, solutions, or emulsions in oily or aqueous vehicles. In addition, the compositions may contain formulator agents such as suspending, stabilizing and/or dispersing agents. Alternatively, the active ingredients may be mixed in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In certain embodiments, it may be desirable to administer the pharmaceutical compositions of the invention locally to the area in need of treatment. This may be achieved by, for example, and not limited to, local infusion during surgery, topical application, e.g., in conjunction with a wound dressing after surgery, by injection, by means of a catheter, by means of a suppository, or by means of an implant, said implant being of a porous, non-porous, or gelatinous material, including membranes such as sialastic membranes, or fibers. In one embodiment, administration may be by direct injection at the site (or former site) of a malignant tumor or neoplastic or pre-neoplastic tissue.

For topical application, the compounds may be combined with a carrier so that an effective dosage is delivered based on the desired activity.

In addition to the formulations described previously, the compounds may also be formulated as a depot preparation. Such long acting formulations may be administered by implantation (subcutaneously or intramuscularly) or by intramuscular injection. For example, the compounds may be formulated with suitable polymeric or hydrophobic materials, e.g., as an emulsion in an acceptable oil, ion exchange resins, or as sparingly soluble derivatives as a sparingly soluble salt.

The compositions may, if desired, be presented in a pack or dispenser device, in which one or more unit dosage form contains the active ingredient. The pack may, for example, include metal or plastic foil such as a blister pack. The pack or dispenser device may be accompanied by instructions for administration.

### Example 3

### VEGF Axis in Prostate Cancer

### (1) β2M Activates the PKA-CREB-VEGF Axis in Human Prostate Cancer Cells

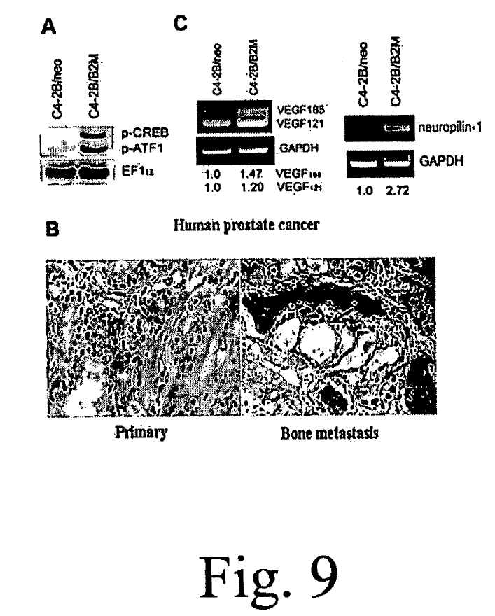

Phosphorylation of CREB/ATF1 in C4-2B cells may be altered by ectopic expression of β2M (FIG. 9A), indicating that activation of the CREB pathway may be a downstream event of β2M signaling. Two molecular pathways may control the functions and survival of prostate cancer cells. First, PKA-CREB may regulate VEGF expression in prostate cancer cells and the ingrowths of endothelium into the cancer tissues by promoting endothelial cell proliferation, motility, and vascular permeability. Second, PKA-CREB activation may mediate AR action to enhance the androgen-induced proliferative responses of prostate epithelial cells and their survival under suboptimal concentrations of androgen as in castrated hosts. β2M may thus be a gate-keeper or switch that selects either AR or growth factor-mediated signaling for prostate cancer cells. By delineating the PKA-CREB-VEGF axis in prostate cancer cells and understanding how the β2M signaling regulates this axis may help predict prostate cancer bone metastasis and provide a therapeutic target for treating prostate cancer bone metastasis.

VEGF overexpression may be associated with increased tumor growth and metastatic spread. Human VEGF monomers have at least five different isoforms of 121, 145, 165, 189, and 206 amino acids. VEGF121 and VEGF 165 may be the most abundant forms. VEGF165 may be partially retained by cells, while VEGF121 may be completely released. VEGF165 may be a more potent endothelial cell mitogen than VEGF121. Expression of both VEGF165 and VEGF121 is increased in β2M-overexpressing C4-2B cells (FIG. 9B). VEGF protein secretion is elevated in β2M-overexpressing (6.2% in total proteins of conditioned medium) versus neo- (3.9%) C4-2B cells. VEGF promoter contains CREs. Treatment with the PKA agonist Forskolin (10 μM) increases VEGF expression. Transiently transfecting C4-2B cells with the expression plasmids encoding either wild type CREB (WT-CREB) or a mutated inactive form of CREB (K-CREB) increases VEGF expression in WT-CREB- but not K-CREB-expressing or control non-transfected cells. These results support the idea that VEGF may be a target gene downstream from CREB signaling.

VEGF binds with high affinity to the tyrosine kinase receptors VEGFR-1 (Flt-1) and R-2 (Flk-1m/KDR), which are expressed on the cell surface of endothelial cells. A third receptor, neuropilin-1 (NP-1), is primarily the coreceptor for VEGF165. Though some prostate cancer lines express Flt-1 and Flk-1, the signals of either receptor expression are not detectable in C4-2B cells. NP-1, however, is expressed in C4-2B cells (FIG. 9). Recent studies show that overexpression of both the VEGF165 isoform and NP-1 correlate with the advanced prostate cancer and a high Gleason grade. NP-1 expression is higher in β2M-expressing C4-2B cells, suggesting a coordinated regulation of both VEGF and NP-1 expression in prostate cancer cells. It also suggests that an autocrine loop is activated to support prostate cancer progression. In addition, VEGF may also have paracrine functions that regulate endothelial cell functions and subsequent neovascular sprouting. However, transient expression of wild type CREB or K-CREB (a mutant CREB) may not alter NP-1 mRNA expression in C4-2B cells, implying that β2M may regulate NP-1 transcription via certain CREB-independent pathway(s). By determining the mechanism by which an autocrine VEGF-NP-1 axis is activated by β2M signaling may help understand how prostate cancer cell grow, survive and migrate in bone microenvironment.

### (2) Bioluminescence Imaging (BLI) of Prostate Cancer Metastasis in Bigenic and Immune-Compromised Mice

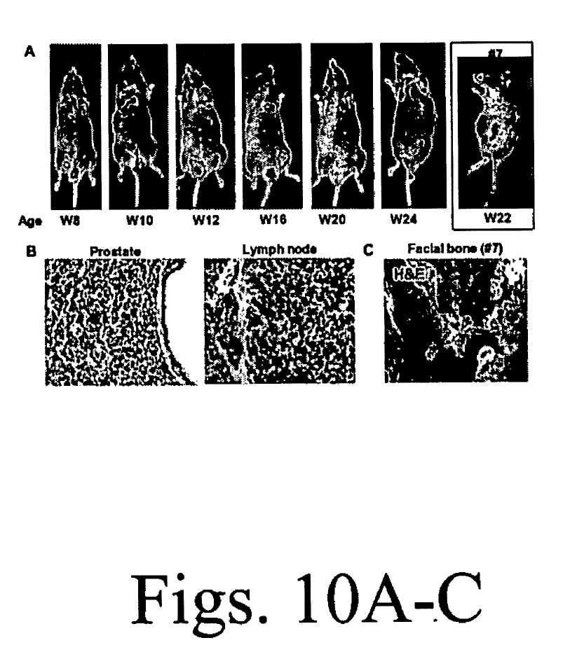

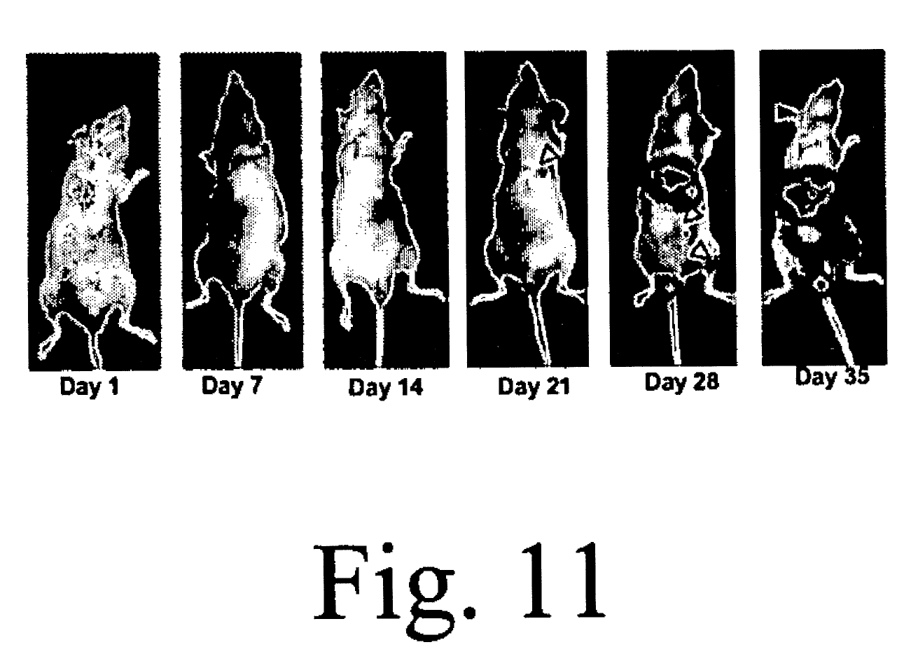

To detect bioluminescence in transgenic animal models and luciferase (Luc) tagged human prostate cancer cells, a Xenogen CCD camera may be used to generate images of metastatic prostate cancer cells. A supra-PSA driven Luc transgenic mice (sPSA-Luc) are generated (J. Mol. Endocrinol. 2005, In Press). The homozygous sPSA-Luc male mice with FVB background crossed with a heterozygous TRAMP female mouse with C57BL/6]F1 (designated as TRAMP-Luc or a transgenic strain overexpressing T Ag in mouse prostate gland) are monitored at 2-week intervals for the appearance of metastatic prostate cancer using a Xenogen CCD camera at 8-24 weeks of age. Mice with visible tumor burdens displayed similar kinetic profiles of BLI. Light emission peaked in the lower abdomen, upper abdomen, chest and groin at 10 to 14 weeks, and then markedly decreased after week 16 (FIG. 10A). IHC staining of SV40-T Ag confirms the tumor distribution in imaged tissues including prostate gland and pelvic lymph node (FIG. 10B). These mice have low incidence of metastasis to jaw bone (FIG. 10C) in a 18-week old TRAMP-Luc mouse. Since there is no detectable Luc-positive metastatic foci in other organs, these results confirm the previous reports that AR is lost in TRAMP mouse prostate cancer cells upon disease progression. Another method of detecting metastatic prostate cancer cells in mice is by injecting the Luc-tagged human prostate cancer cells via intracardiac route. PC3M-Luc cells (5×10⁴) are injected into the left ventricle. The mice bearing human cancer cells are monitored by a Xenogen CCD camera. FIG. 11 shows the prostate cancer cells detected within minutes by BLI (Day 1) into the left ventricle after injection. In all mice, as early as 2 weeks after injection, metastasis is detected in various tissues including liver, adrenal gland and left tibia (FIG. 11, indicated by arrows).

### Example 4

### (1) Regression of Human Prostate Cancer Grown in Nude Mouse Femur or as Bone Powder Implants by Intralesional Administration of β2M siRNA Liposome Complex

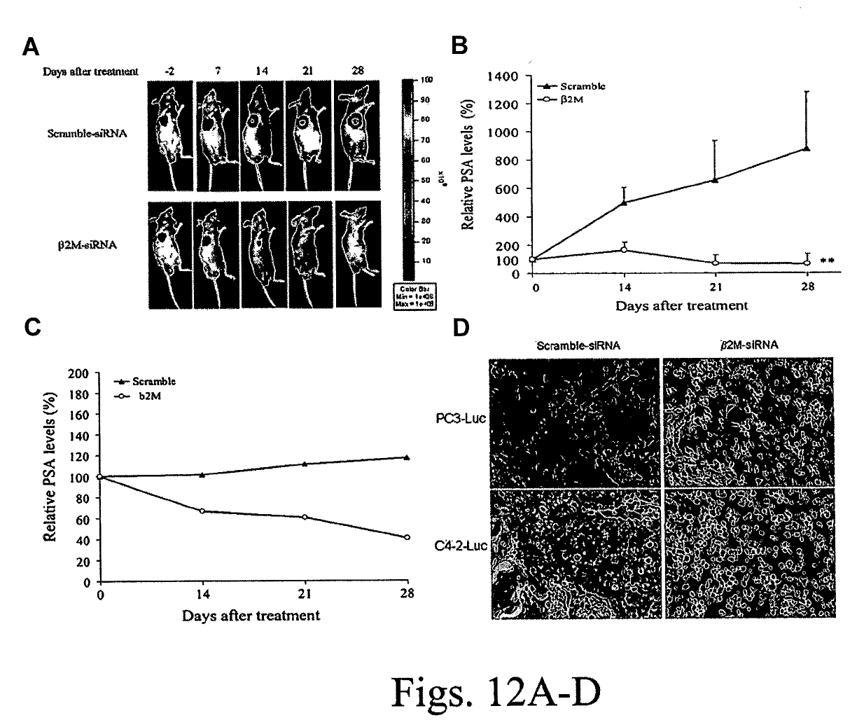

The siRNA liposome delivery and αv integrin activity in mouse bone harboring human prostate cancer have been demonstrated. Since β2M is a mitogen and a signal molecule in human prostate cancer cells, downregulating β2M using a siRNA approach may inhibit the growth of pre-established prostate cancers in mouse bone. Two model systems are used. First, a bone powder model pioneered by Dr. Hari Reddi at UC Davis, where he and Charles Huggins in the 1970s showed that an acellular rat bone powder preparation implanted subcutaneously in syngenic or athymic animals recapitulated complete bone morphogenesis and cyto-differentiation including the ability to form osteoclasts, osteoblasts, mineralized bone, bone marrow and red blood cells, by recruiting host cells. Using this model, PC3-Luc or C4-2-Luc grow in bone powder, form highly interactive prostate cancer cell clusters with bone cells. Upon β2M siRNA-liposome treatment, prostate tumors regress dramatically as assessed by Luc-imaging (PC3-Luc) or serum PSA (C4-2-Luc model) (FIGS. 12A and B). Similarly, β2M siRNA-liposome treatment is highly effective in inhibiting the growth of C4-2 prostate tumors in mouse tibia as revealed by serum PSA (FIG. 12C). These results are confirmed by the massive tumor cell death present in tumor histomorphology in bone powder (FIG. 12D). In addition, liposome encapsulated with siRNA against αv integrin or β2M is not associated with toxicity in host animals judging by the body weight of the mice and their level of physical activity. These results are consistent with the fact that β2M knockout mice develop mild level of autoimmune disease without major consequence on their organ development and postnatal growth.

### (2) β2M Activation Alters Integrin Isotype Expression and Depresses AR-Mediated Signaling

β2M siRNA treatment causes changes in cell attachment to ECM proteins in human prostate cancer cells. In particular, C4-2B cells are treated with either β2M siRNA or scramble siRNA followed by analyzing their attachment to ECM proteins such as collagen I (Col I), laminin (LM), fibronectin (FN) and collagen IV (Col IV). Cell attachment to BSA coated wells serves as an internal control (Con). FIG. 13 shows no significant difference between the adhesion of C4-2B cells transfected with β2M siRNA and scramble siRNA in the attachment to Col I, LM and FN. However, a decreased attachment of C4-2B cells to the basement membrane Col IV is observed in β2M siRNA treated cells. Col IV is known to play a role in supporting prostate cancer growth and survival. These results suggest that β2M could affect α1β1 and α2β1 integrin expression on cell surfaces. These data also reveal the prostate cancer cell attachment to collagen matrices is another consequence of β2M targeting that may ultimately affect tumor growth and survival in bone.

Downregulation of AR and PSA expression is another consequence of targeting β2M signaling in prostate cancer cells. FIG. 14 shows that AR and PSA protein expression, as assessed by Western blot analysis, is abolished in β2M siRNA treated but not in parental and scramble siRNA infected C4-2B cells. These results are not caused by cell selection because β2M siRNA transfected cells are selected by antibiotic resistance and not by single-cell cloning. Despite the marked decrease of AR in β2M siRNA treated C4-2B cells, β2M siRNA-liposome treated prostate cancer continues to synthesize and secrete PSA, suggesting an incomplete antagonism of AR. Possible androgen, growth factors, and/or cytokines may induce PSA expression via residual AR in these cells. The implications of these results include: (1) downregulating β2M expression may effectively eliminate or attenuate AR signaling, thus, removing one support for prostate cancer growth and survival in bone; and (2) downregulating β2M expression may also decrease VEGF signaling and block AR from supporting prostate cancer cell survival, thus, controlling prostate cancer growth in bone.

### Example 5

### Determination the Effect of β2M Overexpression and Signaling on Bone Metastasis in Human Prostate, Breast, Lung, and Renal Cancer

β2M-(high, intermediate and low expressing clones) and neo-transfected human prostate (C4-2B), lung (H358), breast (MCF7) and renal (Rcc) cancer cells are selected. β2M mRNA and protein expression are determined by RT-PCR and ELISA, respectively, with the established procedures in the laboratory. The growth and metastasis of these human prostate cancer cell lines in mice may be tested in three models (15 mice/group/tumor type): Model 1, direct injection of 1×10⁶ cells intrafemorally in mouse bone and the growth and survival of human prostate, lung, breast, and renal cancers in mouse bone may be monitored bi-weekly and non-invasively by radiographic methods; Model 2, direct injection of 1×10⁶ cells into previously implanted bone powder to evaluate cancer cell growth and in situ interaction with newly formed bone; and Model 3, direct injection of 1×10⁶ cells into the left ventricle of mice followed by monitoring the metastatic spread of the cancer cells. Models 1 and 2 allow assessing the ability of cancer cells to grow, survive, and colonize in bone microenvironment. The difference between Models 1 and 2 is represented by adult and newly formed bone, respectively. The advantages of Model 2 may include tumor growth in bone, and that may be followed using luciferase imaging. Multiple implantations of tumors are possible, and the tumor growth in bone powder may be assessed in a time-dependent manner. The advantage of Model 1 may include the tumor growth as bone xenografts mimicking closely cancer cell growth in bone. Model 3 allows the study of multi-step nature of cancer growth and dissemination to bone, although a longer latent period may be expected. If cancer growth and dissemination to bone is closely mimicked by β2M protein expression, the stably transfected β2M- and luciferase-expressing cancer cells may be used to further monitor the time dependent spread of cancer cells to bone (such as time course, location of the skeleton, tumor-stroma interaction) by intracardiacally injecting these cells and monitoring them using a CCD camera. The growth, dissemination, and local bone osteoblastic and osteolytic reactions of cancer cells in bone may be determined by histopathology and IHC (e.g. expression of growth, apoptosis, osteoblastic and osteolytic biomarkers and differentiation-related genes).

A direct correlation between the steady state level of β2M expression and the ability of cancer cells to colonize in bone may be observed in Models 1 and 2. This may be reflected by the ability of high β2M-expressing cancer cells to metastasize more frequently with short latent period to bone (Model 3). Time course study of cancer cell growth in bone may reveal the infiltration of host inflammatory cells to bone and bone powder. This reaction may be correlated with the levels of β2M expression in tissues. A concordance between cancer growth in bone and the intensities of their radiographic and luciferase imaging may be established. Histopathologic and IHC data may support both the osteoblastic and osteolytic lesions in cancer cell growth in bone with relatively more osteoblastic reactions for prostate cancer cells and more osteolytic actions for breast, lung and renal cancer cells. The infiltrating lymphocytes, mast cells, and macrophages in cancer specimens may be observed. A positive correlation between the status of the infiltrating cells, β2M expression in cancers or cancer metastases, and growth and cancer metastasis in mice may be observed.

### Example 6

### Characterization and Validation of the β2M-Mediated Downstream Signaling Components, in Particular, VEGF Signaling, in Prostate Cancer Cells and Surrounding Cells in the Microenvironment

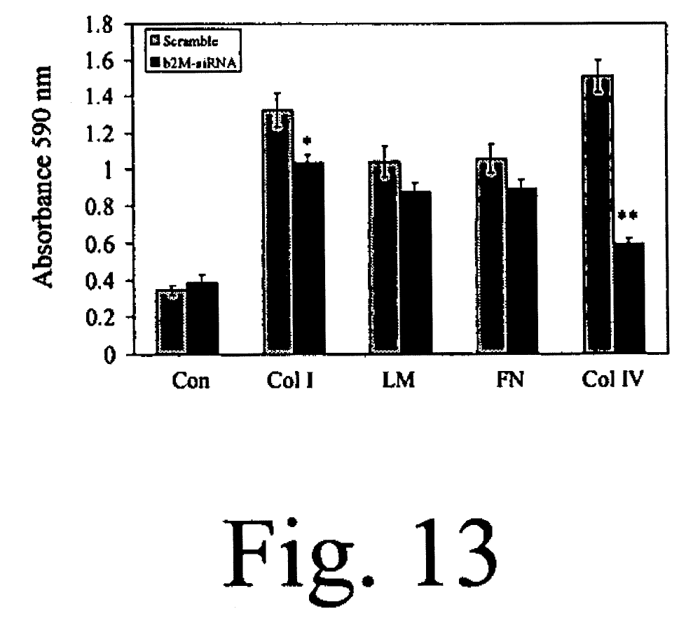

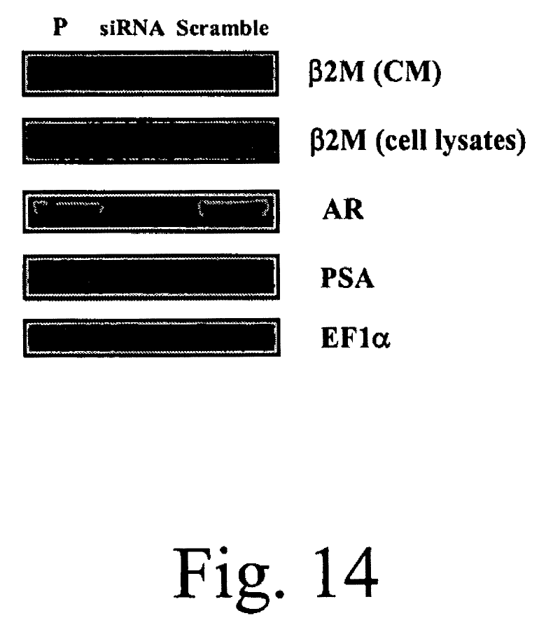

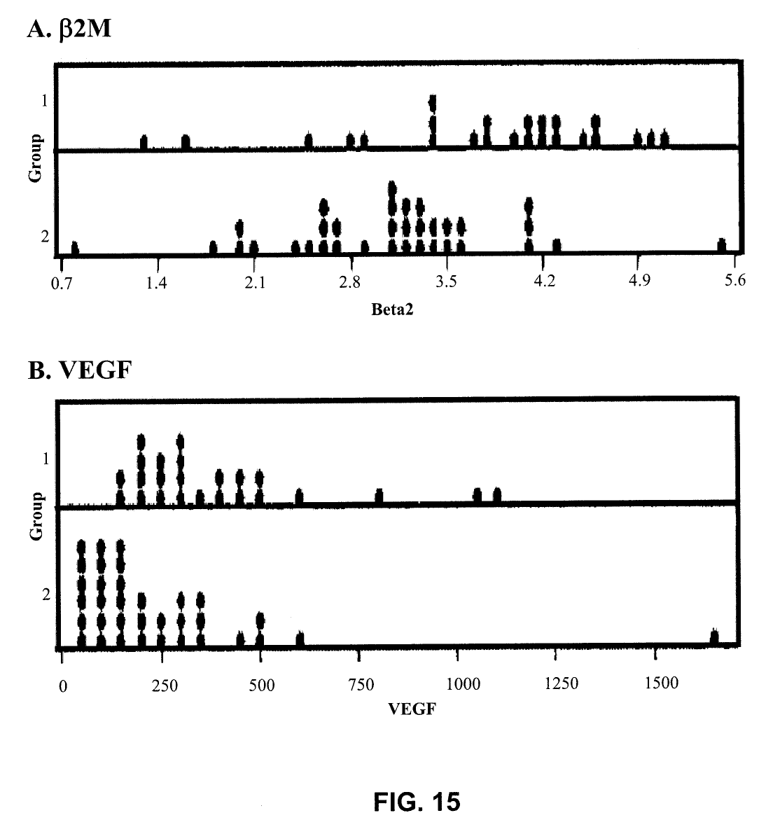

A positive correlation between β2M signaling and the status of activation of PKA, pCREB, osteomimicry, and the activation of VEGF and AR-related growth and survival signaling pathways may be observed. An autocrine loop of VEGF-NP-1 activated by β2M signaling may be demonstrated in LNCaP lineage cells, whereas a paracrine loop of VEGF-VEGFR may be activated by β2M signaling in cancer and endothelial interaction model. Additional biomarkers associated with β2M signaling revealed by microarray (Table 3), and related to the VEGF-NP-1 pathway may be confirmed in the animal models. Upon activation of the β2M signaling pathway, an elevation of the serum markers reflecting increased osteomimicry (OC, BSP, OPN), activated VEGF-NP-1 (increased levels of agonists and decreased their competitors, Sema3A and 3B) and increased AR signaling and EMT (PSA, VEGF are AR target genes whereas vimentin and RANKL are EMT biomarkers), gained integrin receptors (α1β1 and α2β1) and other targeted molecules (Table 3). Serum or bone marrow specimens harvested from mice may reveal correlative values of these markers in predicting prostate cancer skeletal metastases at the time before radiographic or PSA prediction of prostate cancer bone metastasis. Tissue distribution and subcellular localization of the markers may yield significant information on the activation status of signaling molecules (e.g. pCREB and its recruited protein complexes in the cell nucleus), thus increasing the predictive values of the biomarkers identified. Increased β2M signaling and its downstream activity may be a biomarker for poor prognosis. While overlapping of β2M and VEGF is observed in serum samples obtained from men with and without bone metastasis (FIG. 15), by using animal models, it may be possible to determine the possible biomarkers downstream from β2M signaling that leads to differentiation of cancer grown in bone or at visceral organs before radiographic evidence of bone metastasis. Thus, it may be possible to predict tumors in bone even when they are small in size. Further, this may have remarkable value in clinical applications because there are no available serum biomarkers that can predict the presence of small foci of bone metastasis in patients.

A convergence between β2M signaling, the activation of CREB downstream target genes, e.g., VEGF and AR signaling may be observed. In AR-negative prostate cancer cell lines (PC3/DU145, AR negative), the CREB-related genes, but not AR downstream genes, may be correlated with β2M activation. The β2M-induced prostate cancer growth in bone, but not in subcutaneous space, may be related with the interaction between the β2M-mediated signaling and cells in bone marrow including osteoblasts, osteoclasts, marrow stromal cells, endothelial cells, and inflammatory cells. Bone metastasis is a dominant phenotype of clinical human prostate cancer. There may be a “vicious cycle” between the signaling components mediated by altered growth factor, ECM milieu, and increased bone turnover (e.g. through RANKL-RANK interaction) in response to β2M that favors prostate cancer bone colonization.

TABLE 3

| | |
|---|---|
| b2-Adrenergic receptor | STAT1 |
| VEGF | b-Catenin |
| STAT3 | G protein-coupled receptor 56 |
| Glutathione peroxisase, | IGFBP3 |
| PDGF b peptide | IGF2R |
| ADAM17 | Heat shock 70 kDa protein 4 |
| IL-8 receptor b | ADAM15 |
| b2M, IGF2 | Vimentin |
| PSA | IGFBP2 |
| Tumor protein D52 | IGF1 |
| CREB-like2 | Phosphodiesterase 3A. |

### Example 7

### Establishing Animal Models with Altered β2M Status for Evaluating the Effects of β2M on Prostate Cancer Growth and Metastasis to Bone and the Use Thereof

Prostate cancer cells may metastasize to sites with increasing β2M accumulation. A lower incidence of prostate cancer metastasis to bone and soft tissues may be observed in mice with non-functional β2M mutant (or β2M null) expression. Tumor metastases to different anatomical sites may differ depending on the route of tumor cell inoculation. The extent of metastases may be determined by the Xenogen machine under a CCD camera. A positive correlation may be observed between β2M expression and cancer metastases. Further, animals harboring prostate cancer bone metastasis may express high levels of serum surrogate biomarkers related to the β2M signaling pathway. β2M overexpression may increase osteomimicry, thus, allowing prostate cancer adhesion to bone-like matrix proteins, e.g., OC, OPN, BSP, and collagen IV integrin receptors. It may, in turn, lead to overexpression of VEGF and AR by the host cells and the activation of their downstream target genes. These genes include growth and survival related genes and EMT associated genes (Table 3), which may increase the growth and survival of tumor cells in bone. A series of assays for mouse genes may be developed. β2M overexpression may increase the incidence of bone and soft tissue metastases in the bigenic mouse strain with a PTEN knockout background. Luciferase imaging may be helpful in detecting prostate cancer bone and soft tissue metastases. Such metastases may be confirmed by histomorphology, IHC, and biomarkers (serum β2M, OC, VEGF, RANKL) associated with β2M signaling activation in mice. Though PTEN knockout mice have a short lifespan (˜20 months), this strain of mouse is invaluable for observing prostate cancer growth and distant metastases. It is possible that bigenic mice may have shorter than normal lifespan, thus, narrowing the window of opportunity for observing tumor metastases. The alternative approaches may be the use of prostate cancer cells stably transfected with β2M. By implanting the β2M stable cancer cells in these animals, it is possible to observe bone metastasis. The IHC data suggest that such approaches may result in human β2M accumulation in mouse bone. This animal models may then be used to evaluate the bone-homing potential of human prostate cancer cells by injecting cancer cells orthotopically or intracardiacally.

Since other bone and soft tissue derived markers are associated with cancer metastasis (e.g., SDF-1, CRCX4, sonic hedgehog, Wnt signaling pathways, and the heparin bound growth factors), the detection of these potential markers by IHC and/or ELISA may help determine a correlation between β2M and the expression of osteomimicry related and unrelated factors known to support prostate cancer growth and bone (and visceral organ) metastases.

Because conditionally β2M overexpression is restricted to bone, increase MHC presentation may not be observed in mouse prostate tumor. The β2M overexpression by mouse bone may increase an autoimmune reaction against host antigens but may not increase host antitumor immunity. Increased β2M expression in mouse bone may support the growth of prostate tumor in PTEN knockout background by inducing osteomimicry, which, in turn, promotes cell growth, attachment to extracellular substratum, and survival in bone. This may result in efficient prostate cancer bone metastasis. Cancer cells are often deficient in MHC class I presentation despite elevated serum β2M. It is possible that the tight coupling between β2M and MHC in normal cells may be lost in cancer cells.

### Example 8

### Use of β2M siRNA, Ribozyme, and Small Molecules to Target β2M-Mediated Cell Signaling

β2M is available in circulation and also produced locally. Locally produced β2M, rather than circulating β2M, may be responsible for prostate cancer growth in bone. For example, β2M is expressed prevalently in prostate cancer cells, the surrounding inflammatory lymphocytes, and the bone- and prostate-derived stromal cells. In response to β2M, prostate cancer cells in primary or metastatic sites exhibit osteomimicry by expressing highly restricted bone proteins such as OC, BSP, OPN and RANKL, normally expressed by osteoblasts. While β2M immunostaining is strong in prostate cancer bone metastasis, normal bone marrow may not have strong β2M immunostaining. These results support the idea that local (cells surrounding cancer cells) expression and accumulation of β2M may contribute to the osteomimicry of prostate cancer cells in bone. Further, locally produced β2M and its induced osteomimicry may be responsible for prostate cancer growth and survival at metastatic bone sites. Knocking down β2M markedly inhibits prostate cancer grown as xenografts in bone powder or as femur implants as determined by luciferase imaging and serum PSA. In addition, massive cell death is observed using histopathology. This observation may be extended by comparing the efficacy of gene-based and small molecule-based β2M interrupters or interfering compounds.

The G protein-coupled receptor (GPCR)-specific signaling may be used to identify small molecules that specifically targets β2M signaling. These receptors are the targets for >50% of the commercial therapeutic agents including more than a quarter of the 100 top-selling drugs. GPCR Bradykinin (BK) antagonists as well as their bisphosphonate (BP) conjugate mimetics may be screened using the methods for inhibiting the β2M-mediated prostate cancer cell growth. In particular, β2M siRNA may be delivered using liposomes as delivery vehicles to pre-existing prostate cancers via intra-lesional injection. Massive tumor cell death may be observed as the result of β2M siRNA treatment. This form of gene therapy may be improved by using intravenous (IV) β2M siRNA complex with liposome with the expression of β2M siRNA or ribozyme controlled by tissue-specific and tumor-restrictive promoters. These promoters may include OC (directing gene expression in cancer and bone cells) or PSA (directing gene expression in prostate cancer cells). Liposome delivery through IV infusion may also be effective. Improved liposome targeting of tumor cells may be achieved by antibody or other targeting ligands such as RGD peptide, folic acid, PSMA, growth factors, cytokines, or aptamers, conjugated to the liposomes.

### Example 9

### β2M Knockout Tumors

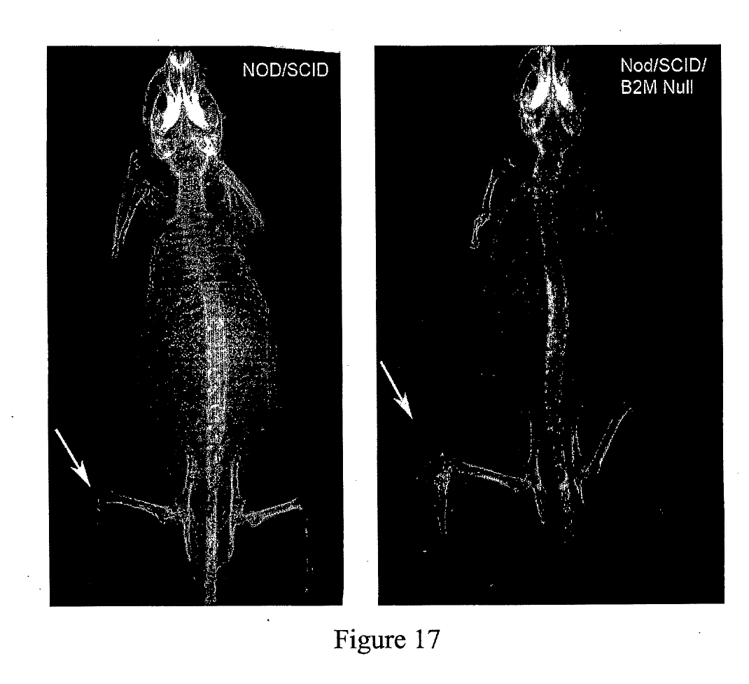

Human prostate cancer cells may grow better in β2M knockout mice than that of the wild type mice. Mice inoculated with one million human prostate cancer cells in bone develop osteoblastic/osteolytic mixed with tumors in β2M knockout SCID mice in less than 2 months (FIG. 17B). Human prostate cancer cells injected in bone in control wild-type SCID mice develop small tumors (FIG. 17A). These results suggest that by inhibiting osteomimicry via knocking down β2M knockdown may increase the colonization of foreign cells including, but not limited to, bone marrow and stem cell transfer to the recipient hosts.

Because the mice with decreased osteomimicry (as a result of β2M knockout) survive and have minimal disease, the transient decrease of osteomimicry may have minimal toxicities in the mouse host. Because cancer cells and benign cells may depend on osteomimicry to maintain their calcification and mineralization potential, decreased osteomimicry may cause massive cell death and decreased cell growth potential.

### Example 10

### Anti-β2M Antibody Treatment Delays Tumor Growth in a Prostate Cancer Xenograft Model

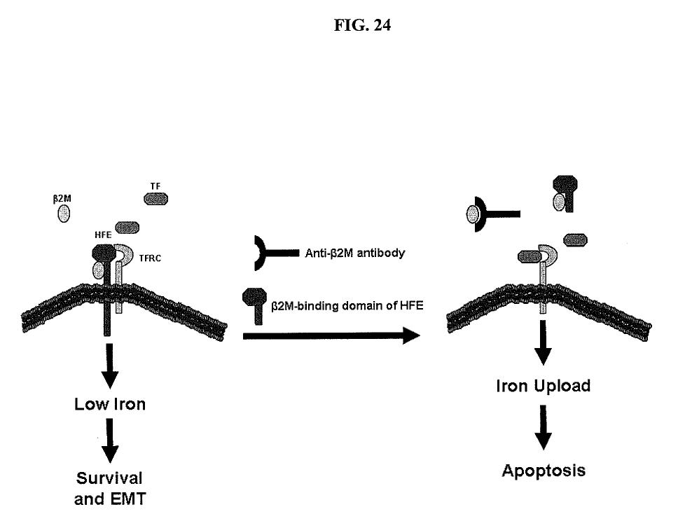

β2M protein forms a complex with MHC class I classical and non-classical proteins. β2M determines the cell surface expression of these proteins. Upon malignant transformation, MHC class I classical proteins are decreased and β2M action may be mediated by the non-classical MHC-like proteins. One such non-classical MHC-like molecule is hemochromatosis gene (HFE), which is associated with a disease manifested by cirrhosis of liver, diabetes mellitus, cardiac arrhythmia and cardiac failure. In majority of the cases, the disease results from a mutation in the β2M-binding domain of the HFE protein. As a result, HFE may not be expressed on the cell membrane. β2M/HFE complex interacts with transferrin receptor (TFRC) and functions as a negative regulator of iron uptake (FIG. 24). Because transferrin receptor is the primary route of iron uptake in most cells, the absence of the β2M/HFE complex in hereditary hemochromatosis patients may increase TFRC activity leading to iron overload.

TFRC activity may be crucial for cancer cells due to a high iron requirement for cell division. Anti-β2M mAbs may increase TFRC activity by inhibiting β2M/HFE complex formation (FIG. 24). As a result, TFRC over activation may lead to iron overload, causing ROS production and increased apoptosis (due in part to reduced DNA repair and increased DNA damage) in cancer cells. Because normal cells have low or undetectable TFRC, anti-β2M mAb may not have adverse effect on the normal cells. Therefore, anti-β2-M mAb may be used to treat cancer cells with minimum side effects.

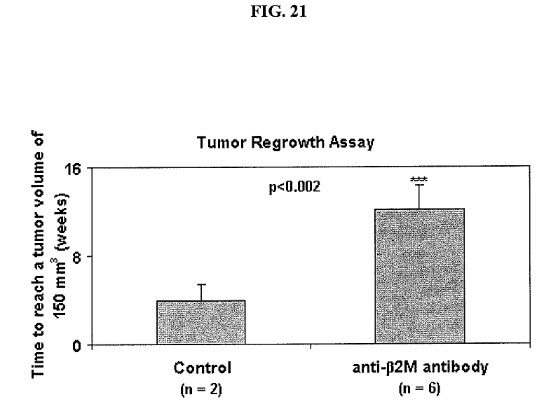

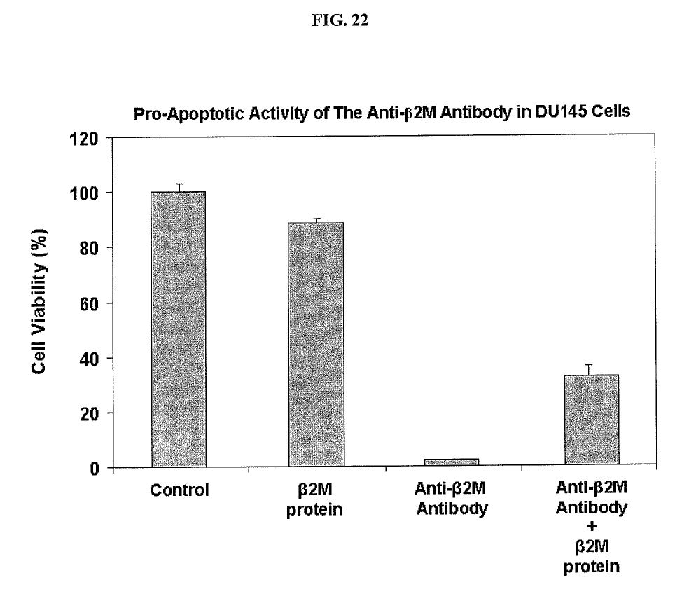

We tested the effects of anti-β2-M mAbs on prostate tumors using a tumor regrowth assay. Briefly, mice are subcutaneously injected with ARCaPM cells on the flank. When the tumor reached a size of 4 mm³, they are treated with IgG or Anti-β2M antibody in Gelform®. The Gelform® is immersed in the 20 μg/ml of antibody and surgically implanted adjacent to the tumors. Tumor volume is measured weekly. The tumor regrowth assay is the time to reach a tumor volume of 150 mm³ after treatment. FIG. 21 shows that the tumors without treatment (control) grow rapidly, whereas the anti-β2M antibody treatment significantly delays the tumor growth. FIG. 22 shows that anti-β2M antibody induces apoptosis in a human prostate cancer cell line, DU145. This apoptotic effect is anti-β2M antibody-specific because β2M protein can block this effect.

### Example 11

### β2M Interacts with HFE

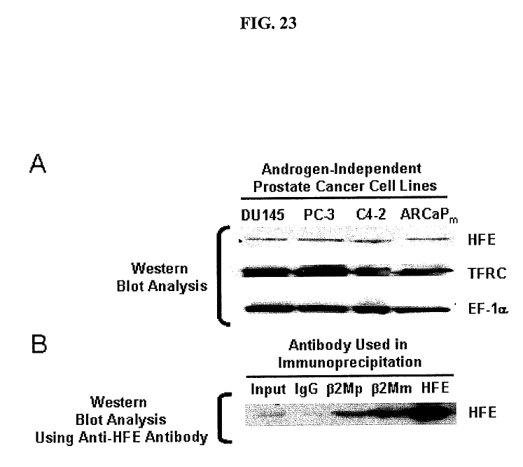

FIG. 23 shows a physical association between β2M and HFE in a co-immunoprecipitation assay. Membrane preparations from DU145, PC-3, C4-2, and ARCaPₘ cells incubated with anti-β2M antibodies (polyclonal (β2 Mp) or monoclonal (β2Mm)), IgG, anti-HFE antibody, and no antibody (input); followed by Western blot using anti-HFE antibody. FIG. 23B shows that β2M binds HFE using β2Mp or β2Mm antibody. FIG. 23A shows that HFE and TFRC are expressed in these cells at a comparable level. EF-1α serves as an internal loading control. These results indicate that β2M binds to HFE. These data also suggest that the β2M-binding domain of HFE may prevent β2M from binding to HFE, thus, leading to iron upload and apoptosis in cancer cells (FIG. 24).

Embodiments of the invention may include one or more of the following advantages. The multi-functional osteomimicry-interfering drugs, e.g., β2M siRNA, β2M antisense, small molecule inhibitors of β2M transcription/translation, anti-β2M antibodies, and β2M-binding domain of HFE protein, may block cancer progression by inducing apoptosis, inhibiting neovascular endothelial sprouting and cell growth, preventing EMT, preventing cancer cell from attaching to ECM, and reducing tumor survival. Further, these drugs may decrease calcification and mineralization of normal benign cells, but induce apoptosis in BPH and fibromuscular smooth muscle cells. The present invention reveals the potential mechanisms underlying the β2M-mediated signaling pathways and identifies the target genes in cancer cells. By targeting the β2M-mediated signaling pathways, new therapies may be developed to treat human cancer, e.g., prostate cancer, with bone metastasis. Novel biomarkers may be identified to screen patients with undetectable bone metastasis for early treatment. Similar approaches may be applied to other cancer types such as breast, lung, and renal cancers that depend on the β2M-mediated signaling pathways for growth, survival, and metastasis.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method for interfering with osteomimetic properties of a cell, comprising:
introducing into the cell an osteomimecry-interfering compound, wherein said osteomimecry-interfering compound prevents or ameliorates the expression of the osteomimetic properties of said cell.

2. A method of claim 1, wherein said cell is a prostate cancer cell.

3. The method according to claim 2, wherein said osteomimicry interfering compound inhibits one or more determinants governing prostate cancer bone colonization, wherein said determinants comprise prostate cancer cell adhesion, extravasation, migration, and interaction with bone cells or a combination thereof.

4. The method according to claim 1, wherein said osteomimicry interfering compound increasing calcification, mineralization and/or bone turnover by modulating the expression of genes restricted to osteoblasts and epithelial to mesenchymal transition (EMT), wherein said osteomimicry interfering compound modulating the expression of one or more bone-like protein selected from the group consisting of osteocalcin (OC), bone sialoprotein (BSP), SPARC/osteonectin (ON), osteopontin (OPN), and the receptor activator of NFκB ligand (RANKL).

5. The method according to claim 1, wherein said osteomimicry interfering compound is one selected from the group consisting of a β2M siRNA, an anti-β2M antibody, a GPCR antagonist, a PKA/CREB signal activation interrupter, an agent interfering with β2M/PKA/CREB signaling, an agent interfering with CREB transcription, phosphorylation, and complex formation, an agent interfering with β2M complex formation with an intracellular protein or with a membrane receptor, a β2M-binding domain of HFE, and a combination thereof.

6. The method of claim 5, wherein said osteomimecry interfering compound comprising the anti-β2M antibody.

7. A method for treating or ameliorating an osteotropic-related cancer or disorder in a subject, comprising administering to the subject an osteomimecry interfering compound.

8. The method of claim 7, wherein said cancer or disorder is selected from the group consisting of osteosarcoma, prostate, breast, colon, lung, brain, multiple myeloma, thyroid, melanoma, and any other disease and disorder with calcification potential.

9. The method according to claim 7, wherein said osteomimicry interfering compound increasing calcification, mineralization and/or bone turnover by modulating the expression of genes restricted to osteoblasts and epithelial to mesenchymal transition (EMT), wherein said osteomimicry interfering compound modulating the expression of one or more bone-like protein selected from the group consisting of osteocalcin (OC), bone sialoprotein (BSP), SPARC/osteonectin (ON), osteopontin (OPN), and the receptor activator of NFκB ligand (RANKL).

10. The method according to claim 7, wherein said osteomimicry interfering compound is one selected from the group consisting of a β2M siRNA, an anti-β2M antibody, a GPCR antagonist, a PKA/CREB signal activation interrupter, an agent interfering with β2M/PKA/CREB signaling, an agent interfering with CREB transcription, phosphorylation, and complex formation, an agent interfering with β2M complex formation with an intracellular protein or with a membrane receptor, a β2M-binding domain of HFE, and a combination thereof.

11. The method of claim 10, wherein said osteomimecry interfering compound comprising the anti-β2M antibody.

12. The method according to claim 7, further comprising:
administering to the subject one or more antagonist, one or more anti-angiogenic agent, one or more cytotoxic drug, or any combination thereof.

13. The method according to claim 7, wherein the osteomimecry interfering compound comprises a vector comprising:
an osteomimecry interfering regulatory region sequence or a transcriptionally active fragment thereof, and
one or more osteomimecry target genes selected from genes related to or downstream from the VEGF axis, AR axis, GPCR axis, PKA/CREB axis;
wherein said osteomimecry interfering regulatory region sequence regulating an activity of one or more of said osteomimecry target genes.

14. The method of claim 13, wherein said cancer or disorder is selected from the group consisting of osteosarcoma, prostate, breast, colon, lung, renal, brain, multiple myeloma, thyroid, melanoma, any other disease consisting of benign prostate hyperplasis, vascular plaque formation in cardiovascular conditions, disorders with calcification and mineralization potential, and a combination thereof.

15. The method of claim 14, wherein said osteotropic-related disease or disorder is osteoporosis, wherein the osteoporosis is associated with bone turnover mediated by interactions between RANK and RANKL; or
A cancer metastasized to bone, wherein the cancer metastasized to bone is mediated by osteoclastogenesis and osteoblastogenesis through osteomimicry and recruitment of host cells.

16. A method for identifying a compound that modulates the osteomimetic potential of a cell, comprising:
(a) contacting a cell exhibiting osteomimetic potential with a test compound;
(b) measuring expression levels of one or more osteomimetic gene products in the cell in the presence and in the absence of the test compound; and
(c) identifying a compound that modulates the osteomimetic potential, wherein the compound changes the expression levels of one or more osteomimetic gene products in the cell.

17. The method of claim 16, wherein the cell exhibiting osteomimetic potential is the cancer cell selected from osteosarcoma, prostate, breast, colon, lung, brain, multiple myeloma, thyroid, melanoma, and any other known disease and disorder with osteomimetic or calcification potential.


SEQUENCE LISTING (6 sequences)

SEQ ID NO: 1 - DNA, 946 bp, Homo Sapiens
gagctcggatccactagtaacggccgccagtgtgctggaattccccttctgcagggtcaggaggagaatcgtggggccaggagggcagaggcacactccatcttcgtgctcctcacaggccctgctccctgcctgctaagacacagggaagggggcccccacctcagtgcctccctcccttccctgtgcctgtgtacctggcagtcacagccacctggcgtgtcccagaaaccaaccggctgacctcatctcctgcccggccccacctccattggctttggcttttggcgtttgtgctgcccgaccctttctcctgtccggatgcgcagggcagggctgagccgtcgagctgcacccacagcaggctgcctttggtgactcaccgggtgaacgggggcattgcgaggcatcccctccctgggtttggctcctgcccacggggctgacagtagaaatcacaggctgtgagacagctggagcccagctctgcttgaacctattttaggtctctgatccccgcttcctctttagactcccctagagctcagccagtgctcaacctgaggctgggggtctctgaggaagagtgagttggagctgaggggtctggggctgtcccctgagagaggggccagaggcagtgtcaagagccgggcagtctgattgtggctcaccctccatcactcccagggcccctggcccagcagccgcagctcccaaccacaatatcctttggggtttggcctacggagctggggcggatgaccccaaatagccctggcagattccccctagacccgcccgcaccatggtcaggcatgcccctcctcatcgctgggcacagcccagagggtataaacagtgctggaggctggcggggcaggccagctgagtcctgagcagcaaagggcgaattctgcagatatccatcacactggcggccgct

SEQ ID NO: 2 - DNA, 1468 bp, Homo Sapiens
gtggcacatatacaccatggaatactatgcagccataaaaaatgatgagttcatgtcctttgtggggacatgtatgaaattggaaaccatcattctcagtaaactatcacaagaacaaaaaaccaaacaccacatattctcactcataggtgggaattgaacaatgagatcacatggacacaggaaggggaacatcacactctggggactgttgtggggtggggggagtaaggggagggatagcattgggagatatacctaatgctagatgatgagttagtgggtgcagcacaccagcatggcacatgtgtacgtatgtaactaacctgcacacaatgtgcacatgtaccctaaaacttaaagtataataataaaaaaaattaagagaaaaaaagaaaaaaaatgatattcattaatttttgatttctcaagcagacttcgcaactggaggaagaataaaatgactagactaggagaatatgcaaactattaagctagatttccctttataaattaaaaaattagtactttagtttatcaatccattctttgtggtgttggtttcatgaatcatttcaaaaacaatggatcactcctgctagctctagtcattttgttattctcataggaaaaaaattaaatatgaaaatgaatagaaaagatatatatagaagcccaagaaaaatcagctgacctcacatgcacgacaggaaggccacataaatggacaatatacagagatttaatttacaaaacaaaatataaaatctgcctctcagtggtatgattctcaaaagttctaacttttatactcagcatcatgttttagcaactatatgttacaaagtctgaccgacttaatcatatcaactttaatttatgagtcaatgaagtatatttcaggaggaaacatcaaatgatattaaaatattgatggttcatctgctggtttcccttattatttagtttttctttctttttttagctaaactaatgtaaaagttatatctaatgacagcaagctttcctttctttcgacatagtgaaaacttgtgtaattatgaaatttttaaaaggttaaagcctttgttatttattttaattcaaatccagtatattattatacatattcggagcccaaactattcatcttcatctaaaccttcaattaaattccacaatgcaaacctcttggctctagaatcacgtttcttgtttattcaactgagcctgtgtcttgaaaaagtgttgaagtttgggggttttctggtgagaatccacgttctgacatcaccttggtcgtgacagtgattggctgttggaaggcaaagaagagtttatagccagcaagagcaagtgaatgagtgagtgagagggcagaggaaatactcaatctgtgccactcactgccttgagcctgcttcctcactccaggactgccagagggtaagatttaatagaacaacgg

SEQ ID NO: 3 - DNA, 2200 bp, Homo Sapiens
  unsure N position 495, 573: a or g or c or t
gggaaggagacaatagtgtcaacttgggattgcctaaggcaacaacagagcaaaacaagaacgctttggttctctgggtctctgtccctgattgcatagcgggtcattgttgggaaatatttcctcacctggcattccaagaaatggtgagctccacagctgtatatagtcctgtcattaaatacaggagtgttctatcccgctggaattaagaaaattggtagaaccagattgtggtctgaaatcttttttcagaaatgctgccatcgtgtggcactgcggagctatgaccagaagagtcctgtaaagggtcgtatggttcatctcaagatggctgggctccagcataatctattcctataattaattctagcttcatattgaatcattcccgtgggcacagagtaaactacagtaaatcctgtggaaattttgttgtttttagaattttcggacttccctccactaaattgacaacatgacacgcttatgcgngtatgtttaaaggaaaaaaatagtttttagaagcagaaaaaagaagtctattttgcaactttataatctgtgtgcttnctattttatagagatagtcgtcatcttacttattaaaatgggtgcttattacctacaaaccaatcatatcaattcatctggaatacatccaatttaagggagacatatttccccctaccaaatgttcatgaaacctatgaattagctatacactatcactgcaagacattatttaatctatatttatattaaaagtaatatttggcaaaaggaagctgacactttaggactaataaaaaccacaattacttttgcagcaacctaataataaataggaccatttatttttcatctcaattacacacaagtcttaacaataaaggtgtaaggtaaataaatagtgcaatctgcatttcacaactgagaagcaaatgaagataagtaatctcaaggcaatattaaatattttaaaaggacccagagctctgctatccctgaattctgctctaatattcggactttccctgtaattttctttcattcagacaccttttaaatacctagtaaagtgttttttaatacagaaatttttaaaaatgtttttctttttaagtggcctactttacataccttgggagaaaaactagaaaaaaagatgattccaaaatcgaatctgttcctttagaaatgtgcaaaatttccttattgatgcatacaatttaaagatcttacgtctactctcattttaataacctgttcttttaaaggacattacaattcgtgactgcctgcccctcttaaaaatttcataatagttaacacacatatagtccttaagatacgcagagcatttgcatctaatatgtgctaagcattgctagtttaacatactaattcatttaaacccctcaaaaaccccatgacctaggtaatagtattgcatttcatggatgagggaacaaggataggtaggctgggcgatttgcccaaggttgcacaggtcagcagtgacacagcggaattcagaaccacggtctggctcctgaagcagccctctcaagcagtcatccttctctcagtcagaaactgctttacttctgcaacatctagaataaattaccattcttctatttcatatagaattttatattttaatgtcactagtgccatttgtctaagtaacaagctactgcatactcgaaatcacaaagctaagcttgagtagtaaaggacagaggcaagttttctgaactccttgcaggcttgaacaatagccttctggctcttcaataagtacaatcatacaggcaagagtggttgcagatattacctttatgttacttaaaccgaaagaaacaaaaatccattgtatttaattttacattaatgtttttccctactttctccctttttcatgggatccctaagtgctcttcctggatgctgaatgcccatcccgtaaatgaaaaagctagttaatgatattgtacataagtaatgttttaactgtagattgtgtgtgtgcgtttttggtttttttttgttttaaccacaaaaccagagggggaagtgtgggagcaggtgggctgggcagtggcagaaaacctcatgacacaatctctccgcctccctgtgttggtggaggatgtctgcagcagcatttaaattctgggagggcttggttgtc

SEQ ID NO: 4 - DNA, 2313 bp, Homo Sapiens
gaattccttgtactttttttcccttctcagttctgcacttaactcgtctaaaaaaattaaaaaagaatttaagaaaccacaaagctaagctgggtgcggtggctcacgcctgtaatcctagcactttgggaagccaaggcattcggattgcccaagctcaggagttcgagaccagcctgggcaacatgttgaaaccccatttctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccaacaaaactttttgaggaacaaagggaaccaggtattttattaattctcatacctccagagtgttaggcacaaaataaacattcaaccaagacctgttgcactgagcagttcatatataacaggagtgacccaagttgaaacgtagaatcagccctctcataccactttttgccaggtgatcataggcaagttacttagcatctatgtttccttattattaaaatggtcataattacaatgcctaagataagggggttgctgtgaagattattaaatcctcagtaaactttggctattgttactcctatgattatcatcaatatcatcaattaccttatctgttcaatactggtggcacaggtccaccagctagatgtctaatcccttatgtgtctattagtggtacaagtggagtttgagtgggatttttttttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctgcccctgggggccattgggtaattccttggagtctccaggcctcacttgccctctgaacaagaaagaggcctgttctggtcatccctccagcctgtccagccctggcactctgtgagtcggtttaggcagcagccccggaacagatgaggcaggcagggttgggacgtttggtcaggacagcccaccgcaaaaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgaggtatgtgtgacccccgcccagcctttcccttctatagttgcaccaaccccgacacccccgttcacgccgtcagctcgtgtgcaagggagggaagctctgctgaggatgcgcctctcctcccggctccatcacggctccccttaagagcatggccctcggtcctgtctgcctgttgcttttcagaaggtggactcactgtgtaactttgtcttcccttacaggtttacaggaaaataatctcactatgttcttcgggggagcattttctcactctctgtttttctctgtgtctgtctctggtttcagaggctgcctgcctgtcctctttgctccctttgcaaatgtggcagcctcctcctttcctgggaatctgatcccatcacagctgccacagggacctggccagcaaccggagtctgtcctccagatctcggtcaggggttctgttttccaaaaagggactttgcagaacaatcagttgatctctgaaagggaaagggggaggcttcaccattaatccacacctctgggaagcttctgttttcctctaattctcctcactcccaaacaccaccttccgtccccccaatacacaaatttcagcaccattctgcctgaaatggcaccatcacaacctcagtcttgggttaggtgttgttcctgtcctgagttccttgggatggtaaacacaggcagtagcccttagtttatctagatctgaaaacccagacatcagatatcgtcaaccaagacatgggtgtaatgggaggtggagtgtgctgggggagatattctcagaagggggaaagggggaagggaagagggagagaattc

SEQ ID NO: 5 - DNA, 4255 bp, Homo Sapiens
acaccaaatatttataaatataactcacacaaataaaacctctttggtgttctcaaaattttgaagaatgtaaaaggtttgaaaattgctgatctagcaaatgactgaacatgaacagctatagtatttgtacctgcccagcagtgcagcaattccttatccttctcatatctgcactttaattttcctttgacaaatatctctccctcctctcagcccatgacatgaggttcacatggggttaacttaattccctggctcaaaggaaaggtattaaattcagacttgtatccaaccattcctgaagctagacttagccctatttttcaataacatgaaccaatcaattttcacatgagtccaaaataattctatgttaatacactaaggtactaggaaatatagtttgagaaatgttgatccaaacattgtgttatttacagtggagtattgacataaactttgaatcttcaaatatgttctggtgtcttggcatctcttaatacctattagcttacaaggctttcactcaactattttataattttgataatgacttaattgattagttgatatattgttaaaataaatatattaatgaatttatgataaataaggcagataaataagacatgcaattaggaagacatgttaaacaaattgttataataatacaatcactctcagcttaggatagctcctggccactttctctctgggtggtttttactctgggagtagtttaaatcattatctagtagtagtttaaagcattatctttgcctaagagctttcgctgactccccacatttgcattgtactaagagttttctctgactccccacataggtctagaccctagtattataagattctcattgtacttgcactttgccttcaaagtactaatcacggttttgttagtgatttgtgtgatgatttgttgaatctttttttttttcccactagggtgtaagccccatgttccatcttgatcaccatgtttctagcccagtgctggcatatagtgggttctcactaatatatctgtagagtaaatgaagaaatgcatgagtgacatgacaggagaatttaaggatgccatgggagcataaaacagagggagccacctgggtgaggagagctgagaaagacttctggagaggcgacatttgagctgagaaaggaaagacaagtgggagagtcctccaggtgtagaagttggagagatgagcgctccagttaggtagtatttgaagctgatgtagaaaaggagtcttgagccagcttgtgaaggactattggagagttttatttttatttttatcttttttttaatttttgagacagaatcttgctttgtctcccaggctggagtgcagtggcatgattgtagcttactgcagcttcgacctcctgggctcaaacaatccacctatctcagccttctgagtaactgggaccagagatgtgcaccaaaatgcctggctaatttgttcattttttgtaaagatagggtctccctatgttccccaggctattctccatctcctgggctccagtgatcctcacgcctcggccacccaaagtgctgggattatagaagtgaaccactgcgcctggcctattgaaggtttttaatcttcagagtttcgactttatcaacaacacttagaagccaccaaagaattgcaggtatggaaatgacatatacttttgcttttagaagaaaatcctgatcagtgtgcacagaattcttcagggggcaagtgtgattcattctgataagatatagcatggcttagactgggagactggcagaggctttgaagatttctttgctcaaattttattcagcaagtatttaccatgcacctactatagcaggcaacatttttaggaaatggtgaatgttacagaggtgaataatacagcaagagtcgttgaacatatggagtttatctattagttggggagtgaatgttgacaaaggaataagtaaatacataggcaagaaagatacattacctgtgaaacagcagcaggtagactgacagtggagtatctaatacagcctatggaagccagaagatagtgggatgacatttttggagtactagtagaaatgtcatatgaagaactctgtaggaatgtaacatacggtcccatatatgaagctcctgggtcaagtatacctgaacataattcagggatttgagggactttcttgtaacctgaggatcaagatgtcaaggaattaaaaacatgtataaaacattgttgtataaaaacccattaaaaagaatggaagacactatagtaaaatcattgtgggtttagttgttataacacattttaaaaatctttgatcccaatcaatatttataagaaagaagaaatatggaattatttcctgagtcaaggagcagggagagaatgaggaagaagaggaggaggaggagggggaggaggagacaataaacctacttcccaaagttaacaaacaaaaagtgggaagaggtcaaagactacaaggagtagaattaacgtcaattgtttctatgtttgagtctgaaaattttttgtcccttctccaccaacctatatattgatacacatataaatgctaaaggcatttttgaatttgaacagatcattttctttgtatggctgcctttaaaaaaaattcaacctggtcactcttcctcaacatttactgaggtctaagtgttcaatttagaacacatgctttaataactcagagacctgtcatttgtcacaaatcttgcctagagaaatactcattagcgaattaggcagaaagaggatgcaaaataaaaaggcacagtagtcccctgatatccatggaagactggttccaggacaccaccaaacccctccccgcaaataccaaaatccatggatgttcaagtttcttaacatatcatggcatagtatttgcatttaacctacacacatcctcttgtacacttgaaattatctttagattatttataatacttaatagaatgtaaatgctatgtaactagttgtgtatcatttaggaaatgatcacaagaaaaaaagtctacagatgttagtccagacacagccatcctttttttttttttcaaatatttttgatctgtggttcattgcatccacagatgtggaacccatggatactgtgggctaactgtattaataaaaaagtggaaacatcctaagtttcatgggtgtttaaattggtcagcaacttccttctgaagaagtatcagaatttgtgagcaatgttaatatttttgttttctcactaagagccacagttctgaatagaggtttttaaaaagccctagcaaggtttctttagcaatgaaactaacatttaactgtatcatcagcttcgtgttacatctctttcctgactgttgggtgagccctcctcggatgcttgcttctggctacacgcccctttacccttttctctgcactgttttcatctttataaagtcagagttggtgtctataggctctctactgccacattcaagacctgcctcgctcaatgtcaccttcaagatgcagaaatagggatttgggaaggggattgtgaaattttcgaagtcttccaaaatactttgagaaactatatttggaagcactttggggggagaggttggacaggaagggtcttcagagatcatcaaatttaactttctaaatcctaaggaggaaaccgagactccaggatgtgaagtcccttctctaccaaactagaatggatgcaggaggaatgtctgaggtgcaatccttatcctttagcaaaggtgtcctctgcgtcttctttaacccatctcttggacctccagaaagacagctgaggatggcaaggggagtctggaaccactggagtagcccccagcctcctccttggagggcccccatgaaggaggcccttcagtgacagagattgagagagagggagggcgaaaggaaggaaggggagccagaggtgggagtggaagaggcagcctcgcctggggctgattggctcccgaggccagggctctccaagcggtttataagagttggggctgccgggcgccctgcccgctcgcccgcgcgccccaggacccaaagccgggctccaagtcggcgccccacgtcgaggctccgccgcagcctccggagttggccgcagacaagaaggggagggagcgggagagggaggagagctccgaagcgagagggccga

SEQ ID NO: 6 - DNA, 6905 bp, Homo Sapiens
tctagaaaataattcccaatattgaatcccaaagaattcaacatttgggctgtcgtttgaaagataagttgaatttggtcatgaaggaagagaggggggatacaatttcagtaaaaggtaacagcaaggtccaaagacagtcaggtcttcagtagtatggagtatattcagagggagccaagatgtctgatgtgaactaaaaagattggtggttggtaggaggaagaggtgtgagaagaggctgtaaagaaaaattgaaacttgattgtgatggactttaaaggctaggctatgggacttggacatgaatctgcaggccagtgtttgcagactggcgcccataactgtctatcacagcaacacagacatgtgttgtttggcctgcagaggtttggcctgcatgatgattttaaaccatctgaattagtagccatcattttcaaaaatcaagagatgccacattaaaatatggaatgctgctgttcttgaaaataatgaaacatctggaacattgaggccacattcctgactgacagcaatcagttggagctgcgtagtgactgcccactttacatggggcatctgatccctagtcgattacagctgccaccacttccctttatctctctaataccaagctcttttcactcatttttgttacttaagagatatttgggtttgaaacctctgatgcaggtaattgagggttatagagcagaggacagatgctatcagagttgtcttttaagaaagaaccctctgttcttcattttgttgaagatagcctggaagagggcagccaggggagaagttagggctggagctatgagaaagcataagatgagatgatggcttcaacattgaggacagaaagaatattgagatgagaaagtagtccatataagcatctatgcaaaggaaatagcagatgtcctcaaatcagcagaggcaacaactctgaaagtttattcataagcccctcttttcatctccaatccagttcaaatgtaattatttaaattgttcttcactctccttcctggatcatgaatgagctccttaaatgcagggtccacagtgtcctattcatcagtgaattccaagtgcctagcacagagcctggcaaatagtaaatgcttaacaaatattcgttcagtgcatgaattggagtgattctctactttgcctcataagttgaaaaaaggtttattacatacctaaatatgctgaaatcacagggcatttggcaaccccccaaaaccaaaactcccagtttggaaacagaattttaattctgtgaaaataaaatccattcatttattcaaaaaatatttattaaacaatgaccatgtccacaccaggctgagtcctaaggattcaatgatgaacaaaaaccaacatgattcctgctcttaggaaacatacagttcagtgaggaaaacagattgtgagaagtcctccaacaaatactgggtgctattaaaatatattaaaaggtgagtgggtgagggacttgagctagcctaggtggttcaggaagtcttcctggatgtgctgatatgcataggcattaactagataaatagagagaaggatgaaccaacattgcaggtagagggaacagaatatgcaaaggcaggaaggattatggagtcgttggaggacctgaataaaggcccagtgtaagtggatctcagaaaacaggaggaaaggtgtatgagatgagatcagagaggcagatcatgtggggtatggttaatgttttggacttttctattaagagcaatggggagacagtgacaggacttaaacggggaaataatatgaccagattaaactttctaaaaaaccctctatgcaaatatatattgagagttaattattgacaaagattcaaaggcaacaaagtggagagagaatagtattttcaaaaaatggtgccaaaacaataggacatctatattaaaagttgggtatctgtctacaaaacttaattcaaaatggatcacagacctaaatgtaaaactgaaagctatacaacttctggaaggaaaacacagatgggaatctgtgtgatcttgagtttgaaaatgatttattatatctgacaccataatccgtaagttaacataattcataagtgaacaaagtgatgaactggacttcatcagaatttaaaatgtttgtgcttcaaaagacactggtatgataatgaagacaaactacagataagatattgttgaatcatatttctgataaaggaattgtggctcagaatacataactctaaacccccataataaattacaagtagcccaattaaaaaaaaaaaaagagaaaaaatttacagtcttcatcaaagaaagtatcaattgtaaaataagcacatgaaaaatgctctgcatctttattcatggggggatgaaataaaaattaaatgggaaagacacctctaattagaatactaaaattaaaaagactgaccataccaagtattggtgaagtggaaatgtaaaatgatacaatcaacttaggtagatgatttggaagtttcttacaaaagtaggtgtatacctaccctgtgactcacccattccatggctaagtatttacctgagagaaatgaaagaatacatccatacaaagatgtttatacaaatatttatagcagttttatttgtagtagccccaaactgaaaagaacccaaatgtccatcaaaagtgaatggataaacaaagcgtggtacagcaatgcaatagaatactacttagcaataaagaagaatgagctagtgatatacataacagcttaaatgtacatcaaaggcattgtgctcagtgaaagatgcaagtaaaaaaaaaaaagagtacatgctgtatagttccattgacataaaactctggaaagtgaaaaacagtctatactgacagaaagcagatcattggttgcctgaggaggaggagtataggagaggtggagggaaaatgtacaaagtggcacaataaaaacttttggaatcatagatatattcactatcttgattgagtgatgatttcatgagtgcacgtgcgtgtgtcaaaaatgatcaatttatgcaactttaaatatgtgcagtttattgtatatatcaattatacctcagtacggctattaaaaagaaaccctctggctgcacaatgcagaactgattctaggaaagagtggagggaggatgaccatttacagtgctccaggtggaagagaacggtgccttctggaagtgaactaggttggcaacaacagagatgaaataaatgggcagatgtgtgagatacttaggaaataaaacccgatggtcaccattttccaaaggtcagctcatcctggctttccagagcaaagagctagggaagactttattaataaatccctcttgaagttgcagaggaagcttatagcagaaacttactctcaacctgactaatctgagagaacacctctggttccatttgattactaaaaaactgcaaagaacaggaggagaaagaagaagaaagctggtacaaacagtgaacttatataatattaatcaataattgtctcttgttcttaaaagcaatgggaagaaaatgagatttgagctggaagatcagagttcaaaatccaaataaagtatatggccctaatatgcttatagtagttaacctttcctgataatgatataattgttgacagcaccatctttaaaataaaataacatagtaatccttcagatttgtagaagatctttcctgtttacaagtttgttctatacacattatgtcttttaaatgacacactagccttctgagggtaacttatattggcaacagttttcagatgtggaaactgtgaagacaatgttggtgatgtggaagcaacataaactttggagtctttcagacccaggtttgaatgtcagactgctttttattcagagtaacttcagagcattatttctcaccttaattttttttcaggcctctttgtgtctatgtgtcctcttcactcctgtccattgtttcttcagtgatttttgccaccttccttcactgttagtgtgtagacacatagttctcctggctctgagagcctatgttaattccattctaccatcctgccacggcccactcaattcctattgagcaatgctagttgaaagttgtggtgggattaaatgttgcaatgagtattcaaatgaggttgaagtatctacgcattctacttacatatggtgaggtatattcaaggaagctgtagccattaaaatctcaggaaataatttttcacctcctcaggtgaaagggtcttcaggcctttgtgttctggaaggttcatttatagccatttcccaaatgacaatgcgattgatgagtctagagtctagctcaaatagcaatggactggaagactagtttaggttttactaatgtggaacatagaacaaattatgtccttgtttcagcctgttcatctgtgaaatagagcctatcatatccagtcttccttgcctttaggtttgagttaccttctttggtcaaggtaagtaaatgcctatgatgtttggctgtgcacaagataaagctacaacaaagctacaacccatcttttctctgtagaagactcaaaaagcaaaagagacccaggaaaatctcggaatgacttttggaacagagagcctccccagaatcagaagtcaaggaatttaaacatagggaaggcccaggtctctactgacataaaggaaagatgttttcttataggtttcacgtttacattttctctctcttgatcccattcccacttgcatctgccacctttacacagggcttatgggacctcctccacaaaagagcagttgcagtaacccacatcatcctctacgccctggctgtccatcaagaggcgaaaagcagccctatataggttctatccttggatagttccagttgtaaagtttaaaatatgcgaaggcaacttggaaaagcaagcggctgcatacaaagcaaacgtttacagagctctggacaaaattgagcgcctatgtgtacatggcaagtgtttttagtgtttgtgtgtttacctgcttgtctgggtgattttgcctttgagagtctggagagtagaagtactggttaaaggaacttccagacaggaagaaggcagagaagagggtagaaatgactctgattcttggggctgagggttcctagagcaaatggcacaatgccacgaggcccgatctatccctatgacggaatctaaggtttcagcaagtatctgctggcttggtcatggcttgctcctcagtttgtaggagactctcccactctcccatctgcgcgctcttatcagtcctgaaaagaacccctggcagccaggagcaggtattcctatcgtccttttcctccctccctcgccccaccctgttggttttttagattgggctttggaaccaaatttcctgagtgctggcctccaggaaatctggagccctggcgcctaaaccttggtttaggaaaccaggagctattcaggaagcaggggtcctccagggctagagctagcctctcctgccctcgcccacgctgcgccagcacttgtttctccaaagccactaggcaggcgttagcgcgcggtgaggggaggggagaaaaggaaaggggaggggagggaaaaggaggtgggaaggcaaggaggccggcccggtgggggcgggacccgactcgcaaactgttgcatttgctctccacctcccagcgccccctccgagatcccggggagccagcttgctgggagagcgggacggtccggagcaagcccacaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcgcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaacaagaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatg